(19) United States
(12) Patent Application Publication
(10) Pub. No.: US 2022/0249447 A1
(43) Pub. Date: Aug. 11, 2022

(54) CF3-, OCF3-, SCF3-, and SF5-Containing Antibacterial Agents

(71) Applicant: Purdue Research Foundation, West Lafayette, IN (US)

(72) Inventors: Herman O. Sintim, West Lafayette, IN (US); George Naclerio, Lafayette, IN (US)

(21) Appl. No.: 17/606,720

(22) PCT Filed: Apr. 24, 2020
(86) PCT No.: PCT/US2020/029689

§ 371 Date: Oct. 26, 2021

Related U.S. Application Data
(60) Provisional application No. 62/838,961, filed on Apr. 26, 2019.

(51) Int. Cl. A61K 31/4245; C07D 413/04; C07D 417/14; C07D 413/14; C07D 271/113; C07D 409/04; C07D 271/07; C07D 413/12; C07D 277/46; C07D 231/40; C07D 257/06; A61K 31/427; A61K 31/4439; A61K 31/4196; A61K 31/433; A61K 31/426; A61K 31/415; A61K 31/41; A61K 45/06

(52) U.S. Cl. CPC A61K 31/4245; A61K 45/06; C07D 417/14; C07D 413/14; C07D 271/113; C07D 409/04; C07D 271/07; C07D 413/12; C07D 277/46; C07D 231/40; C07D 257/06; A61K 31/427; A61K 31/4439; A61K 31/4196; A61K 31/433; A61K 31/426; A61K 31/415; A61K 31/41; C07D 413/04

(57) ABSTRACT

The present invention generally relates to compounds as a new antibiotic to treat various infections, including infections caused by methicillin-resistant Staphylococcus aureus (MRSA), vancomycin-intermediate and vancomycin-resistant S. aureus (VISA and VRSA respectively) vancomycin-resistant Enterococcusfaecalis (VRE), and Clostridioides difficile. Pharmaceutical compositions and methods for treating those infection diseases are within the scope of this invention.

### CROSS-REFERENCE TO RELATED APPLICATIONS

This present U.S. patent application under 35 U.S.C. § 111(a) relates to and claims the benefits of the U.S. Provisional Application No. 62/838,961, filed on Apr. 26, 2019, the content of which is incorporated herein by reference in its entirety.

### TECHNICAL FIELD

The present invention generally relates to compounds and methods for the treatment of a patient with a bacterial infection.

### BACKGROUND

This section introduces aspects that may help facilitate a better understanding of the disclosure. Accordingly, these statements are to be read in this light and are not to be understood as admissions about what is or is not prior art.

The discovery and development of antibiotics revolutionized health care in such a way that bacterial infections, which were otherwise deadly, could be treated^{1, 2}. However, this was met with a rapid development of resistant bacterial strains that rendered many antibiotics ineffective³. Consequently, millions of people are infected with drug-resistant bacterial strains yearly resulting in thousands of deaths. In the US, the Centers for Disease Control and Prevention in 2013 estimated that approximately 23,000 people died from infections caused by drug-resistant bacterial pathogens at an annual infection rate of about 2 million. The cost to treat such recalcitrant infections exceeds $20 billion per year ^{4, 5}. There are unmet needs to fight various bacterial infections.

### BRIEF DESCRIPTION OF DRAWINGS

FIG. 1: Antibacterial activity of Compounds. Compounds were tested at 16 μg/mL for their ability to inhibit S. aureus growth. The OD600 of compounds were normalized to that of the DMSO control.

FIG. 2: Time-Kill assay results with selected compounds.

### DETAILED DESCRIPTION

While the concepts of the present disclosure are illustrated and described in detail in the description herein, results in the their description are to be considered as exemplary and not restrictive in character; it being understood that only the illustrative embodiments are shown and described and that all changes and modifications that come within the spirit of the disclosure are desired to be protected.

The present invention generally relates to compounds useful for the treatment of an infection diseases. Pharmaceutical compositions and methods for treating those diseases are within the scope of this invention.

In some illustrative embodiments, the present invention relates to a compound having a formula (I)

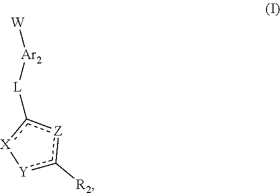

or a pharmaceutically acceptable salt thereof, wherein [symbol]
represents a single or double bond;

X, Y or Z is, independently, CH, O, S, N, or NR, wherein R is H, alkyl, aryl, heteroalkyl, or heteroaryl;

W is —CF₃, —OCF₃, —OCHF₂, —SCF₃, or —SF₅;

L is a linkage selected from the group comprising —NH—, NR—, —NHCO—, —CO—NH—, —NRCO—, —CO—NR—, —CO—, —NH—SO₂—, —NR—SO₂—, —O—, —S—, —S(═O)—, or —S(O₂)—;

R₂ is an optionally substituted aryl, heteroaryl, alkyl (linear, branched or cyclic), heteroalkyl (linear, branched or cyclic), amide, CN, urea, sulfone, sulfoxide, sulfonamide;

and

Ar₂ is an optionally substituted aryl or heteroaryl.

In some illustrative embodiments, the present invention relates to a compound having a formula (I) as disclosed herein, wherein the compound has a formula (II)

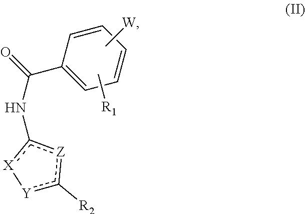

wherein [symbol]
represents a single or double bond;

X, Y or Z is, independently, CH, O, S, N, or NR, wherein R is H, alkyl, aryl, heteroalkyl, or heteroaryl;

W is —CF₃, —OCF₃, —OCHF₂, —SCF₃, or —SF₅;

R₁ represents four substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cyclo alkyl, cycloalkenyl, cycloheteroalkyl, cycloheteroalkenyl, aryl, heteroaryl, arylalkyl, arylalkenyl, and arylalkynyl, each of which is optionally substituted; or any two adjacent substituents are taken together with the attached carbons form an optionally substituted cyclic or heterocyclic moiety;

and

R₂ is an optionally substituted aryl, heteroaryl, alkyl (linear, branched or cyclic), heteroalkyl (linear, branched or cyclic), amide, CN, urea, sulfone, sulfoxide, sulfonamide;

In some illustrative embodiments, the present invention relates to a compound having a formula (I) or (II), wherein X and Y are N, and Z is O.

In some illustrative embodiments, the present invention relates to a compound having a formula (I) or (II), wherein R₁ is hydrogen or halo.

In some illustrative embodiments, the present invention relates to a compound having a formula (I) or (II), wherein at least one of four R₁ is a halo.

In some illustrative embodiments, the present invention relates to a compound having a formula (I) or (II), wherein one of four R₁ is alkoxy or heteroalkoxy.

In some illustrative embodiments, the present invention relates to a compound having a formula (I) or (II), wherein W is —SCF₃ or —OCF₃.

In some illustrative embodiments, the present invention relates to a compound having a formula (I) or (II), wherein W is —CF₃.

In some illustrative embodiments, the present invention relates to a compound having a formula (I) or (II), wherein W is —SF₅.

In some illustrative embodiments, the present invention relates to a compound having a formula (I) or (II), wherein the compound is

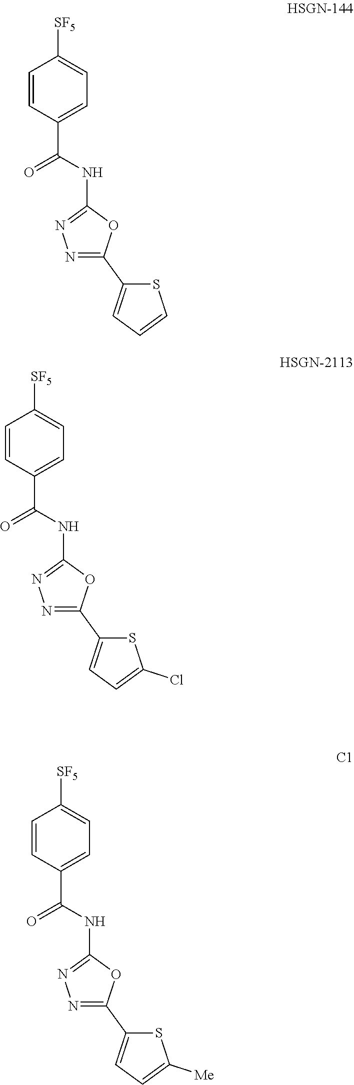

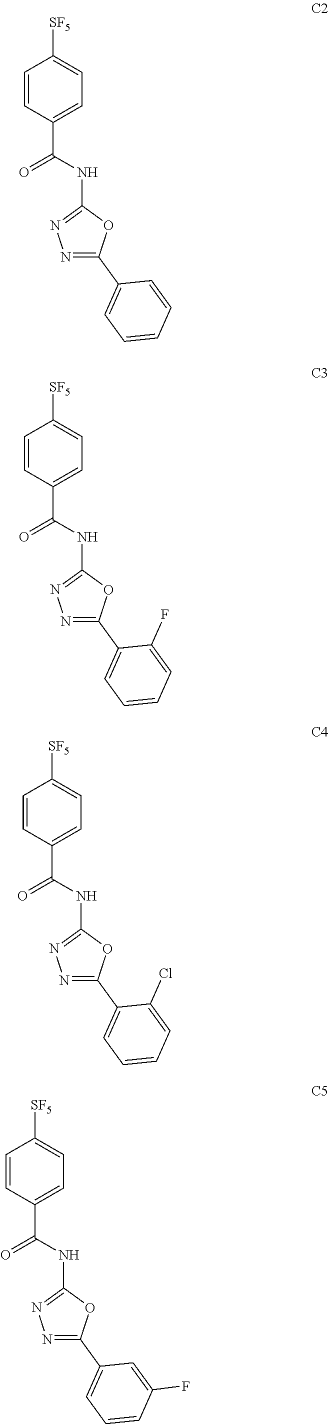

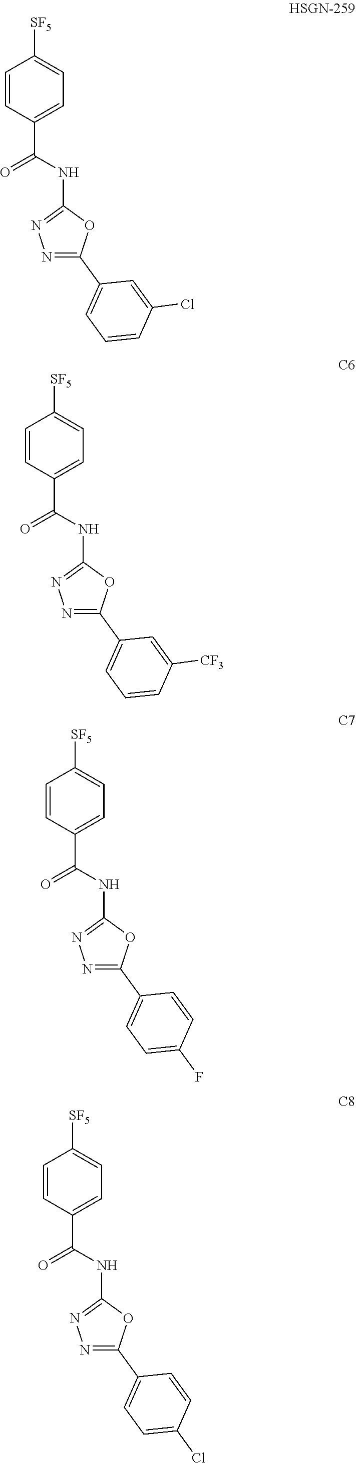

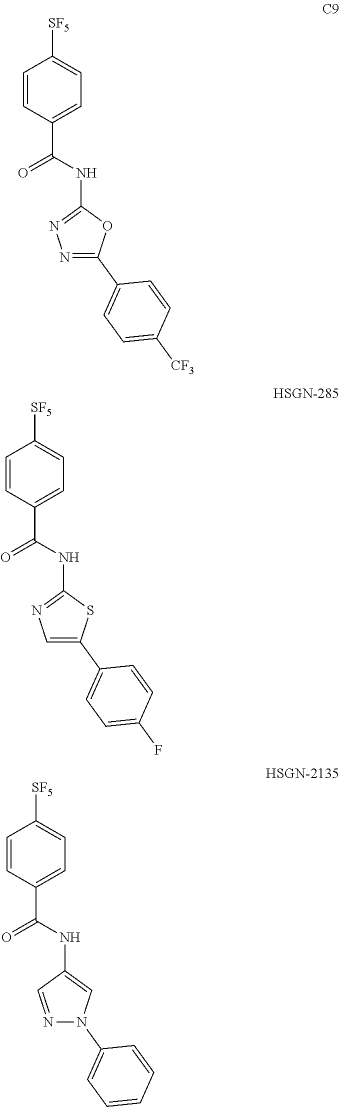

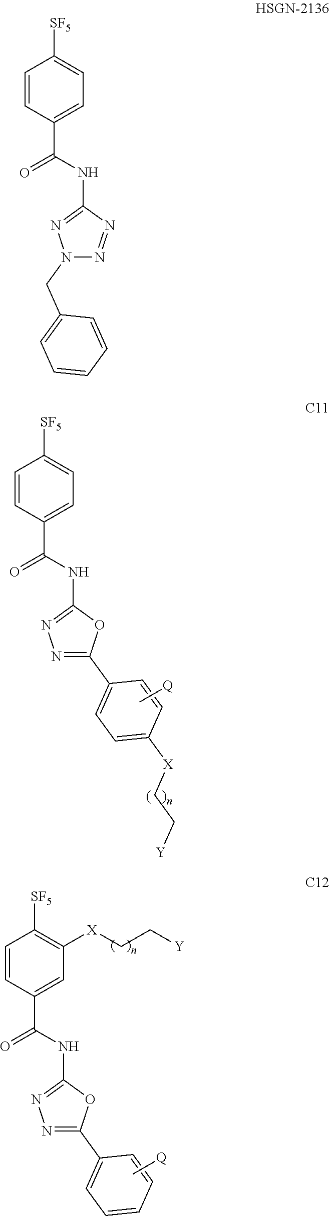

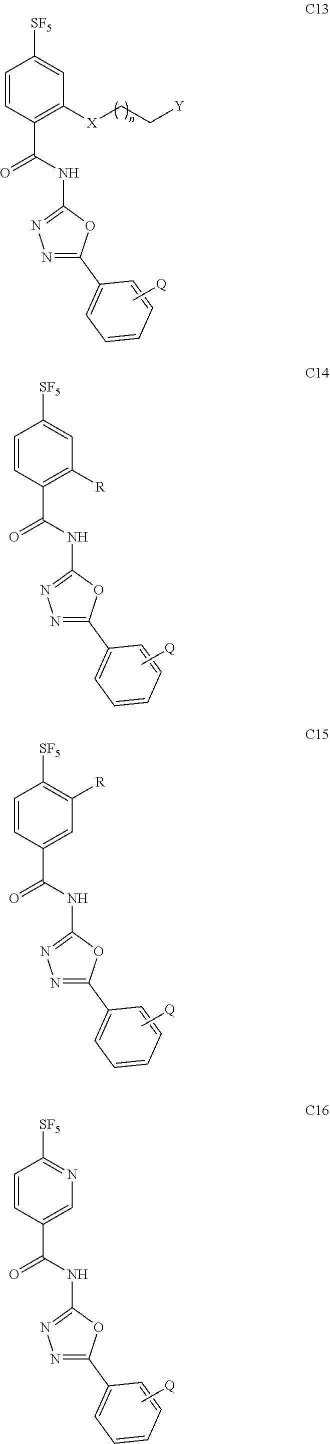

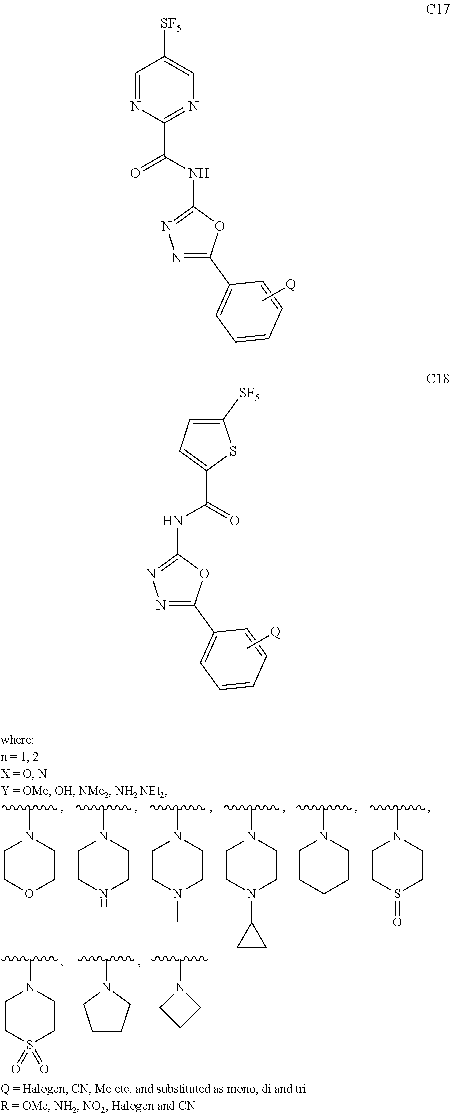

In some illustrative embodiments, the present invention relates to a compound having a formula (I) or (II), wherein the compound is

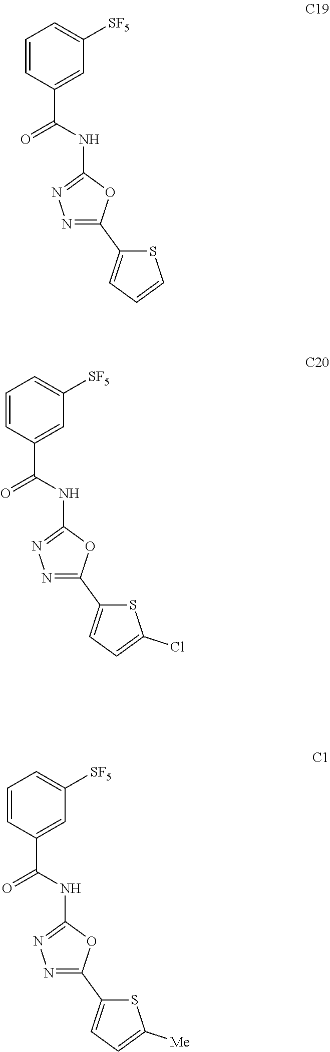

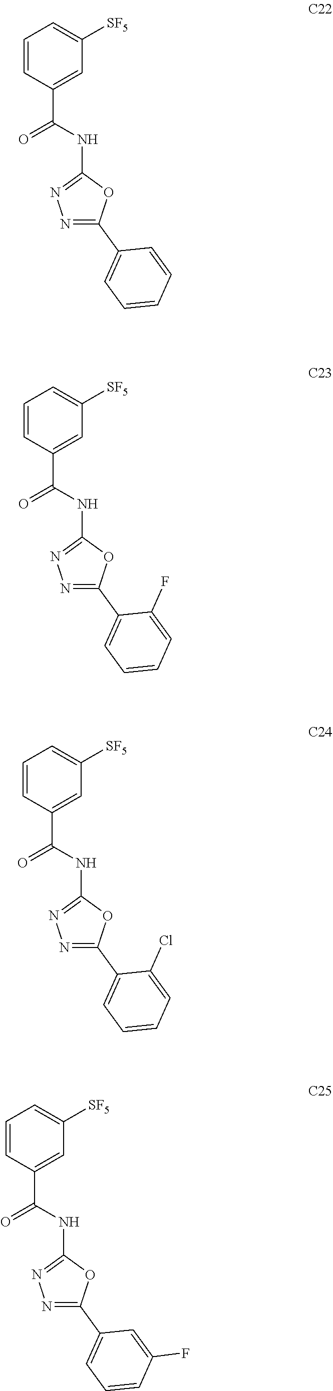

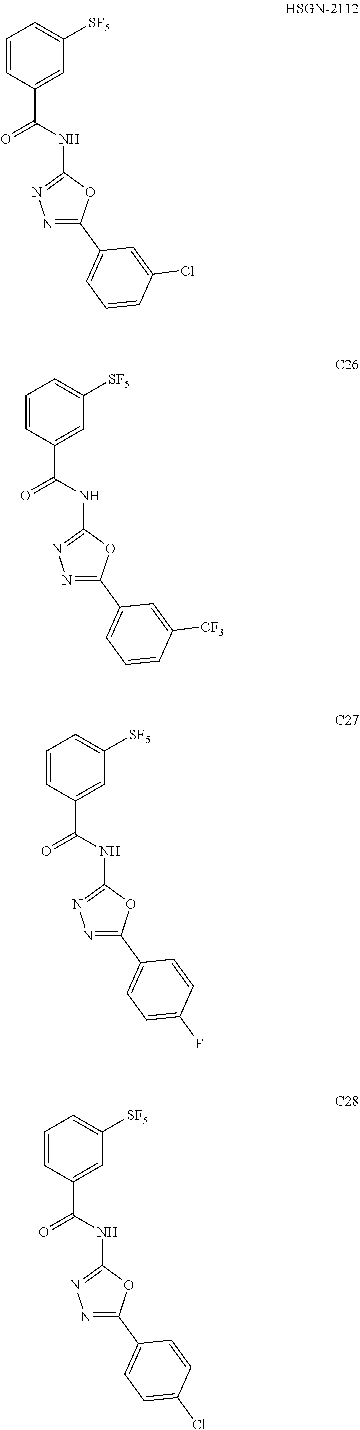

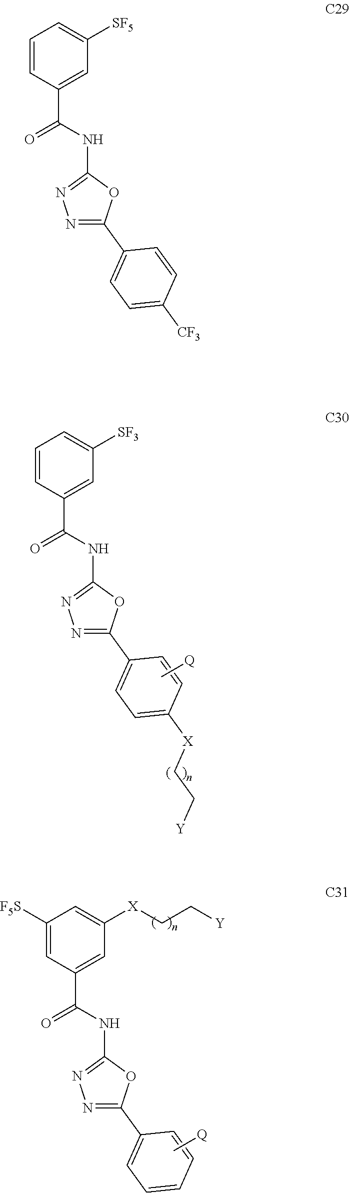

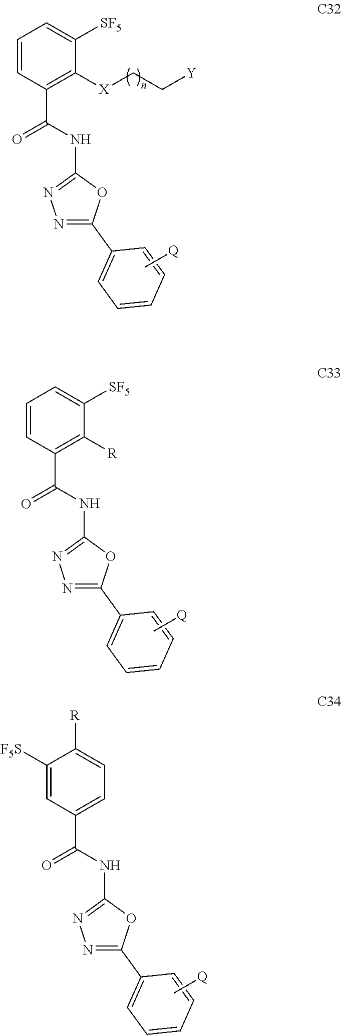

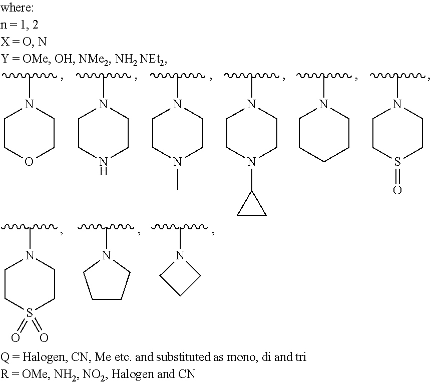

In some illustrative embodiments, the present invention relates to a compound having a formula (I) or (II), wherein the compound is

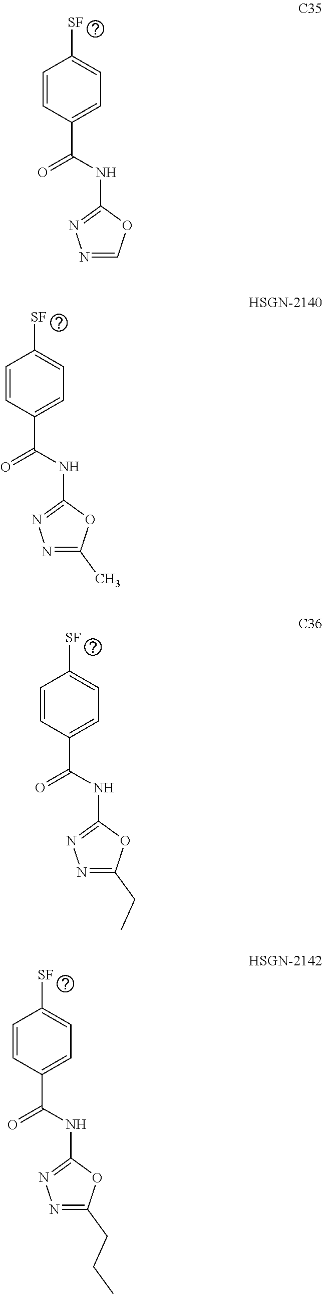

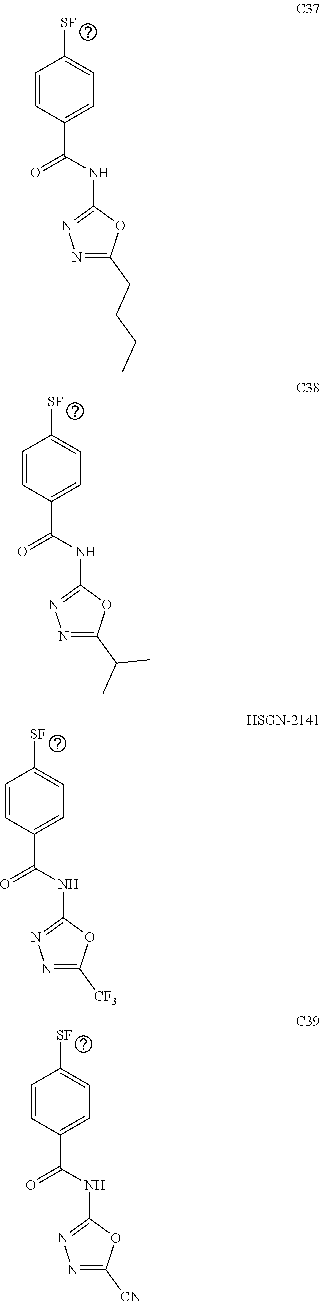

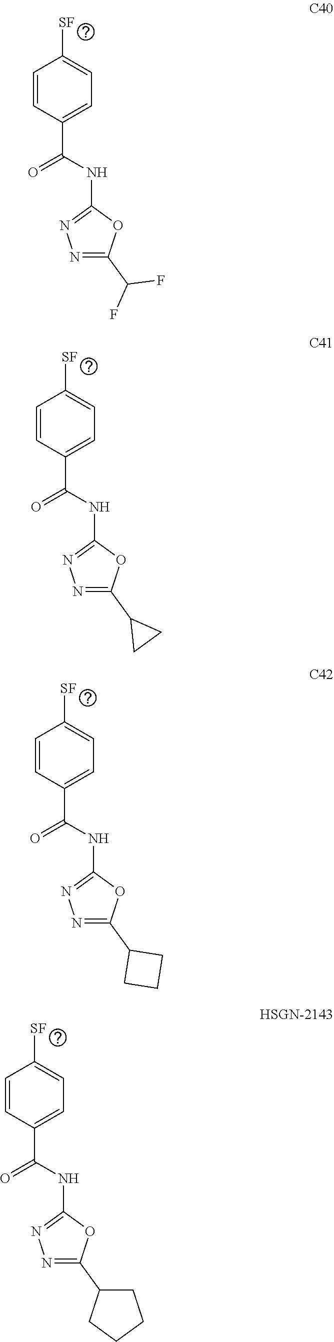

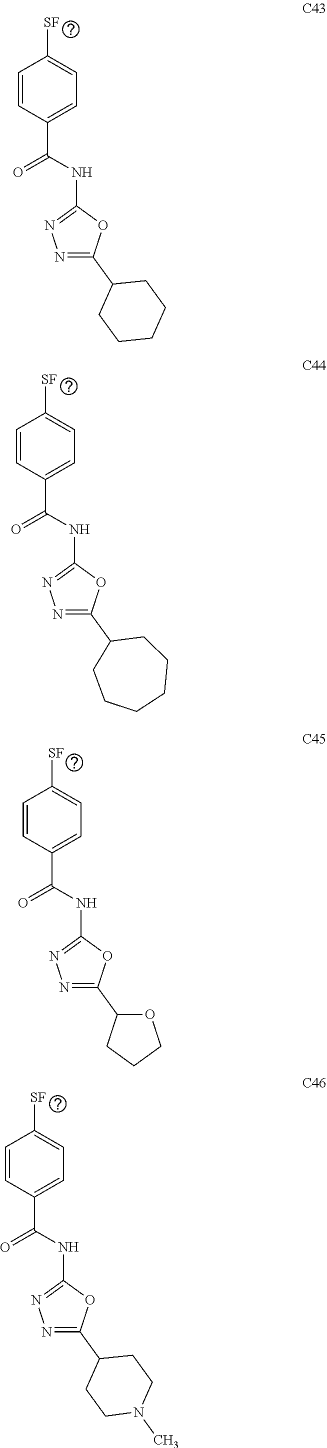

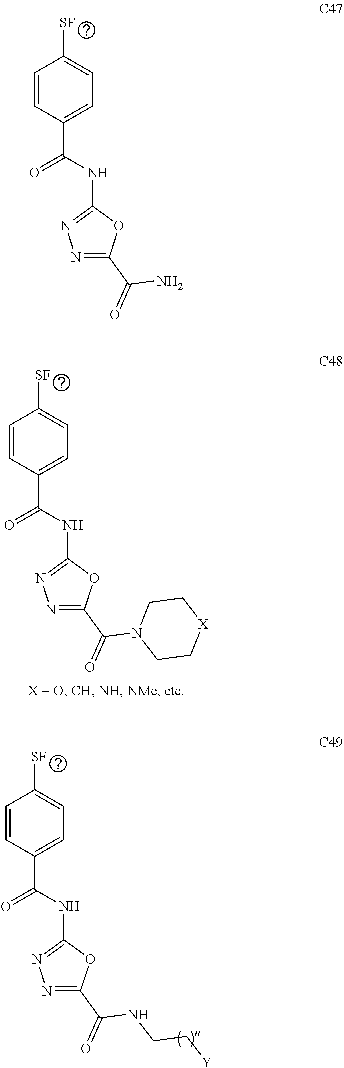

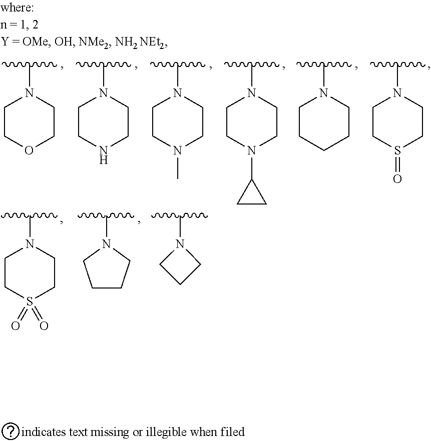

In some illustrative embodiments, the present invention relates to a compound having a formula (I) or (II), wherein the compound is

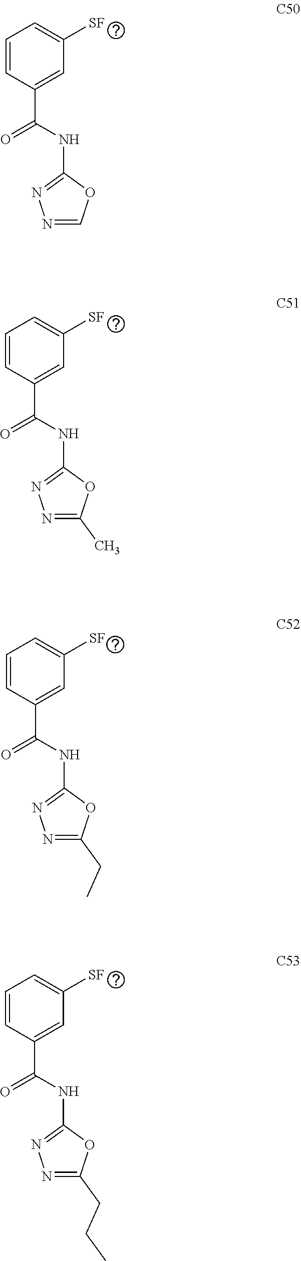

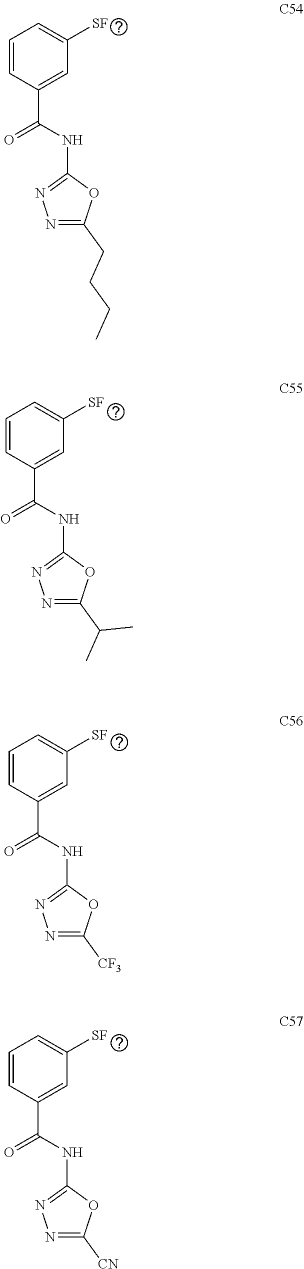

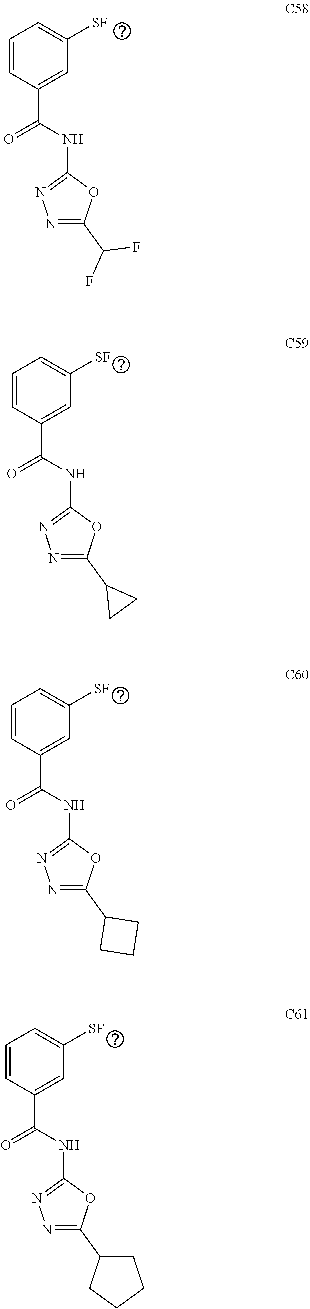

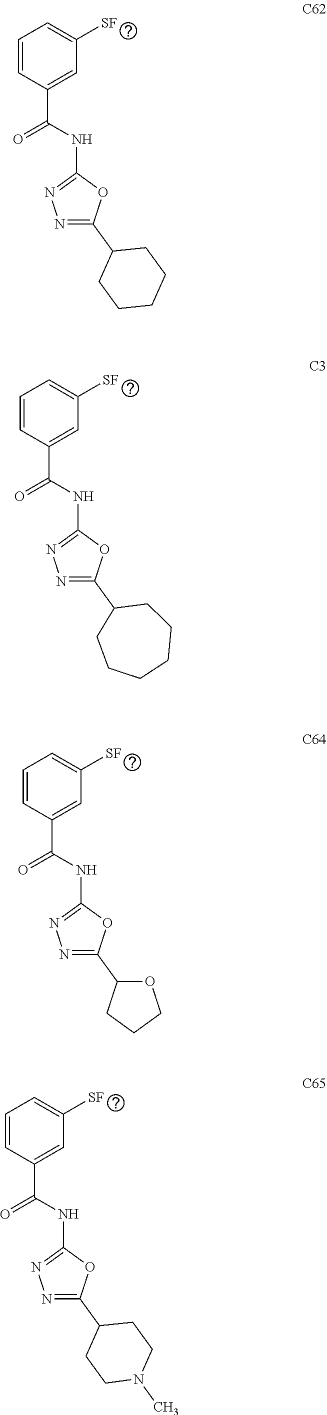

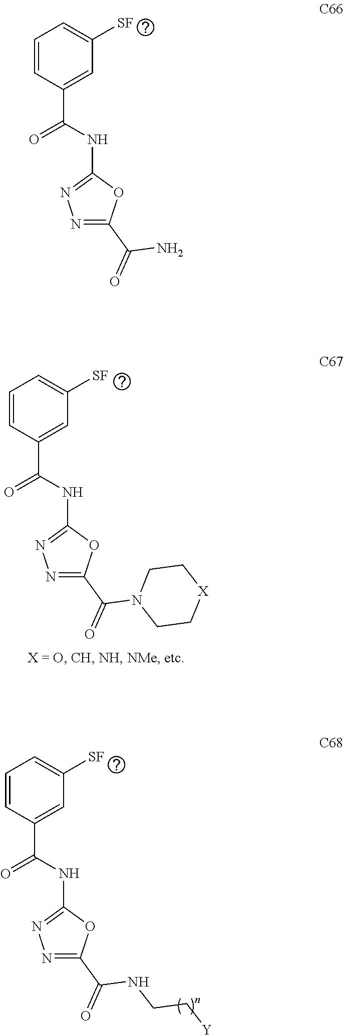

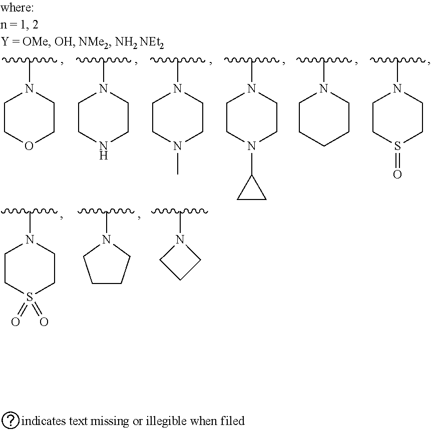

In some illustrative embodiments, the present invention relates to a compound having a formula (I) or (II), wherein the compound is

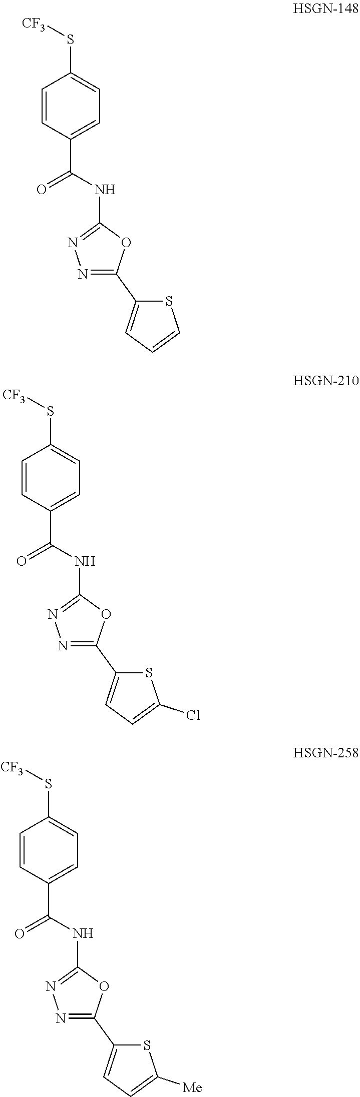 [SMILES: O=C(Nc1nnc(-c2cccs2)o1)c1ccc(SC(F)(F)F)cc1 ; O=C(Nc1nnc(-c2ccc(Cl)s2)o1)c1ccc(SC(F)(F)F)cc1 ; Cc1ccc(-c2nnc(NC(=O)c3ccc(SC(F)(F)F)cc3)o2)s1]

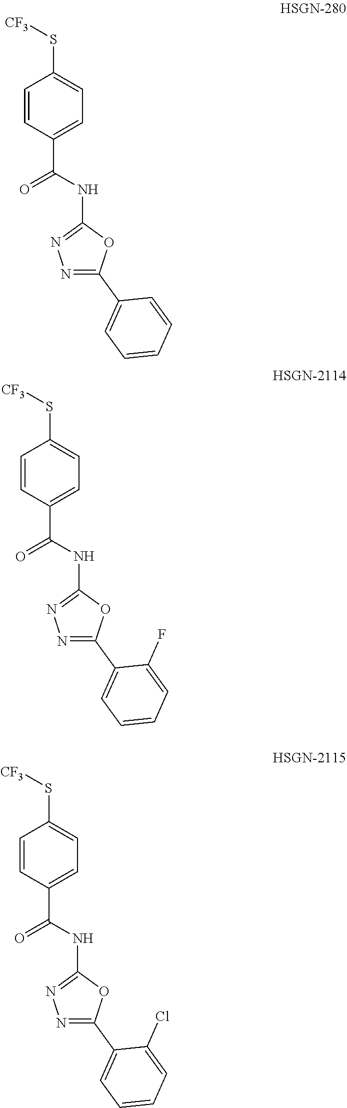 [SMILES: O=C(Nc1nnc(-c2ccccc2)o1)c1ccc(SC(F)(F)F)cc1 ; O=C(Nc1nnc(-c2ccccc2F)o1)c1ccc(SC(F)(F)F)cc1 ; O=C(Nc1nnc(-c2ccccc2Cl)o1)c1ccc(SC(F)(F)F)cc1]

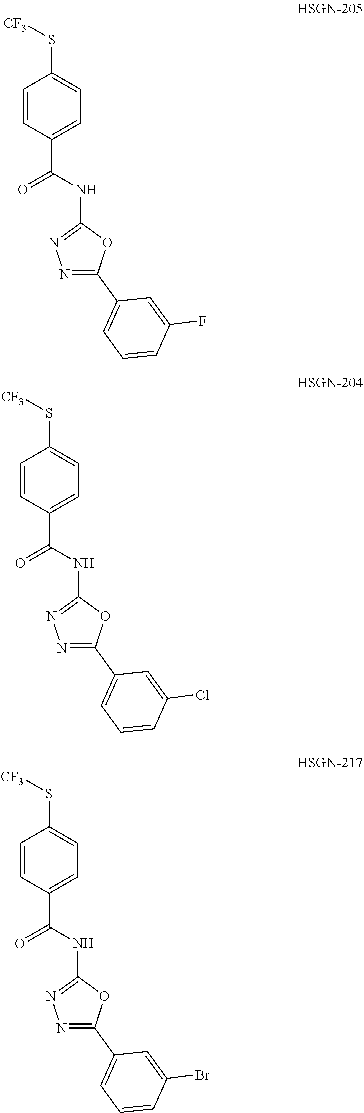 [SMILES: O=C(Nc1nnc(-c2cccc(F)c2)o1)c1ccc(SC(F)(F)F)cc1 ; O=C(Nc1nnc(-c2cccc(Cl)c2)o1)c1ccc(SC(F)(F)F)cc1 ; O=C(Nc1nnc(-c2cccc(Br)c2)o1)c1ccc(SC(F)(F)F)cc1]

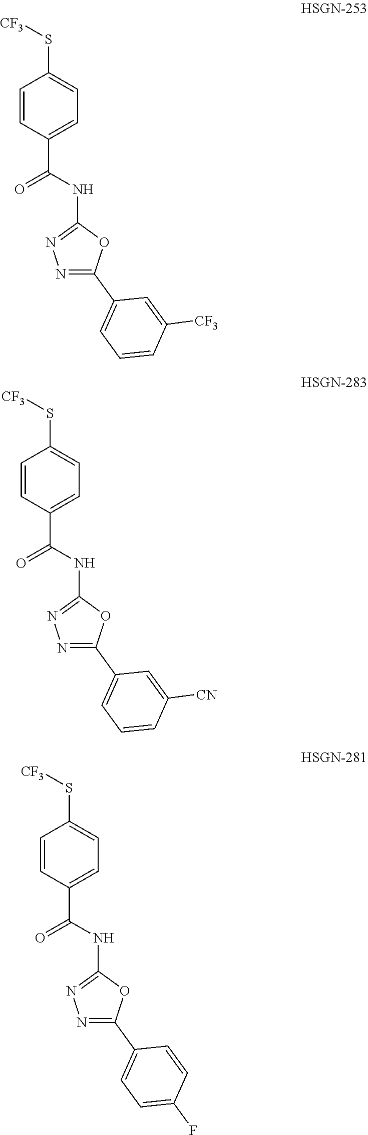 [SMILES: O=C(Nc1nnc(-c2cccc(C(F)(F)F)c2)o1)c1ccc(SC(F)(F)F)cc1 ; N#Cc1cccc(-c2nnc(NC(=O)c3ccc(SC(F)(F)F)cc3)o2)c1 ; O=C(Nc1nnc(-c2ccc(F)cc2)o1)c1ccc(SC(F)(F)F)cc1]

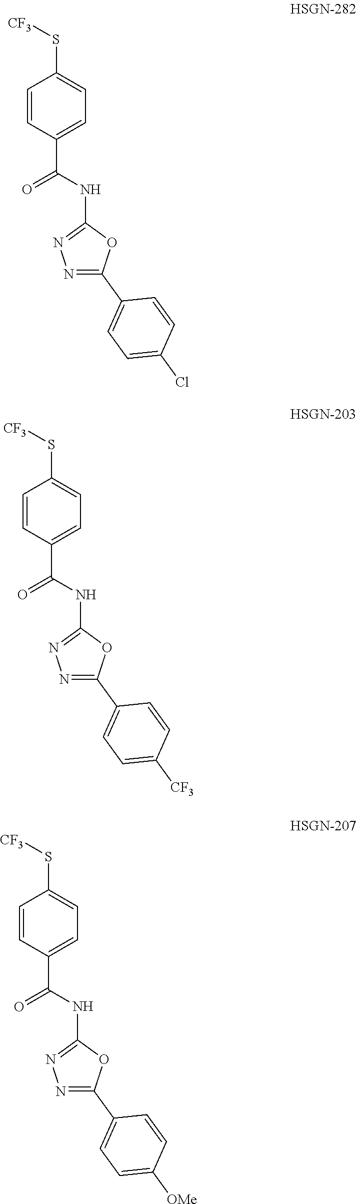 [SMILES: O=C(Nc1nnc(-c2ccc(Cl)cc2)o1)c1ccc(SC(F)(F)F)cc1 ; O=C(Nc1nnc(-c2ccc(C(F)(F)F)cc2)o1)c1ccc(SC(F)(F)F)cc1 ; COc1ccc(-c2nnc(NC(=O)c3ccc(SC(F)(F)F)cc3)o2)cc1]

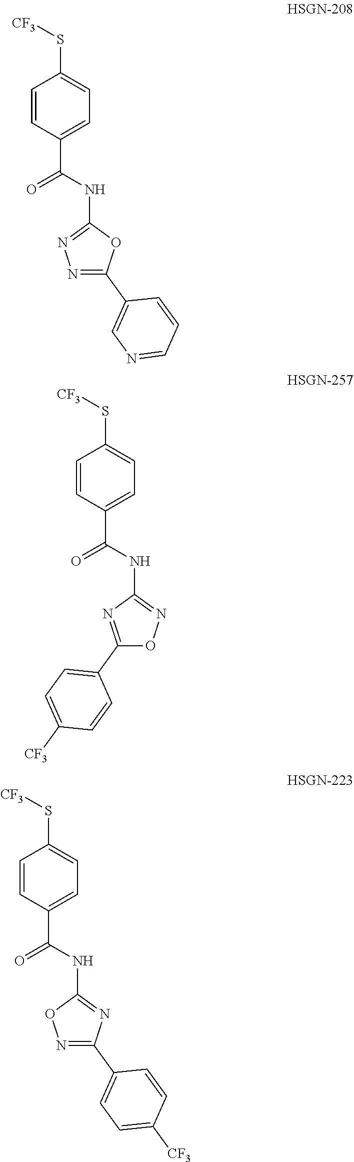

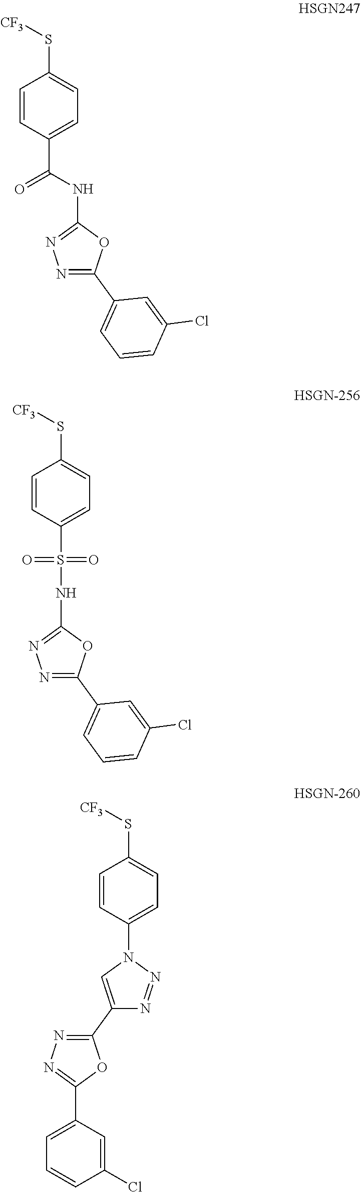

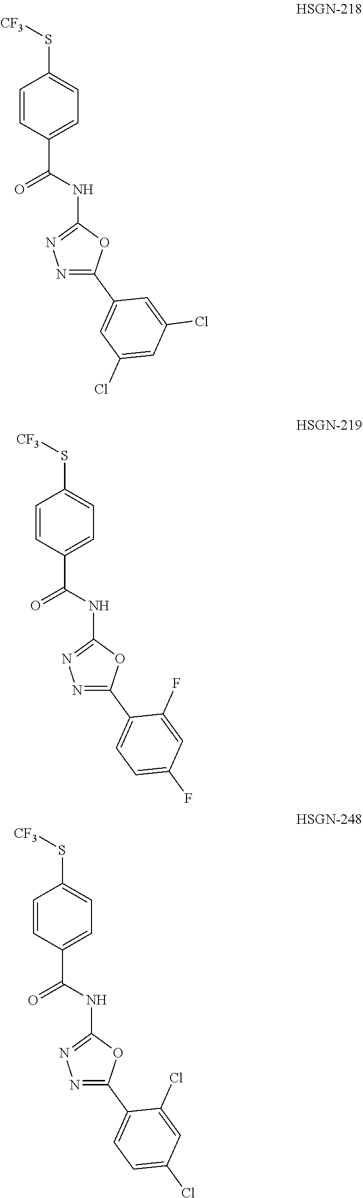 [SMILES: O=C(Nc1nnc(-c2cc(Cl)cc(Cl)c2)o1)c1ccc(SC(F)(F)F)cc1 ; O=C(Nc1nnc(-c2ccc(F)cc2F)o1)c1ccc(SC(F)(F)F)cc1 ; O=C(Nc1nnc(-c2ccc(Cl)cc2Cl)o1)c1ccc(SC(F)(F)F)cc1]

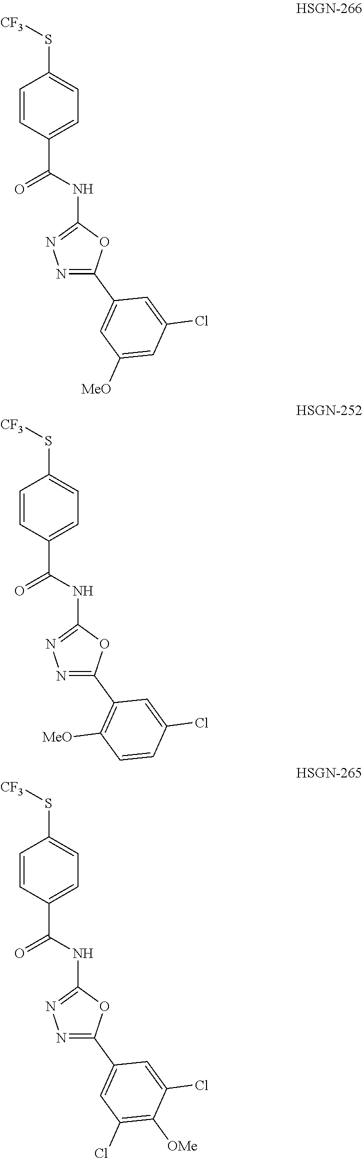 [SMILES: COc1cc(Cl)cc(-c2nnc(NC(=O)c3ccc(SC(F)(F)F)cc3)o2)c1 ; COc1ccc(Cl)cc1-c1nnc(NC(=O)c2ccc(SC(F)(F)F)cc2)o1 ; COc1c(Cl)cc(-c2nnc(NC(=O)c3ccc(SC(F)(F)F)cc3)o2)cc1Cl]

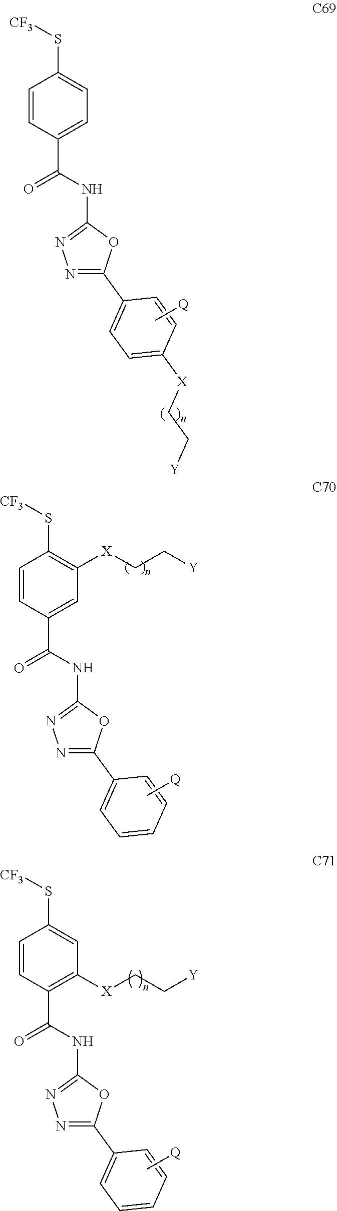

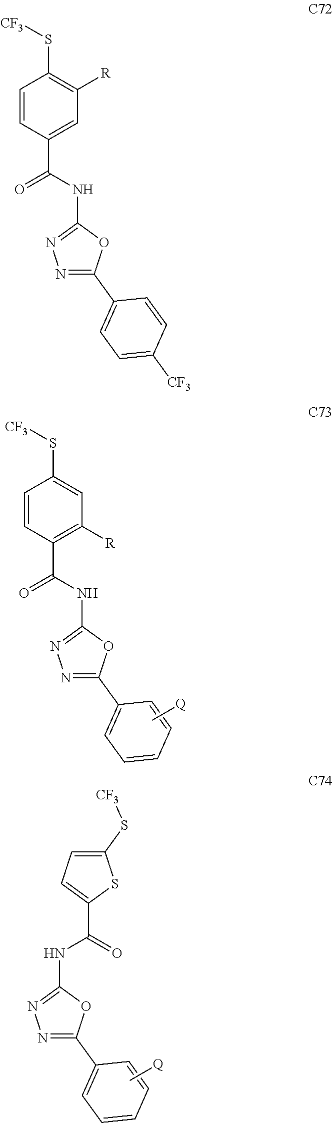

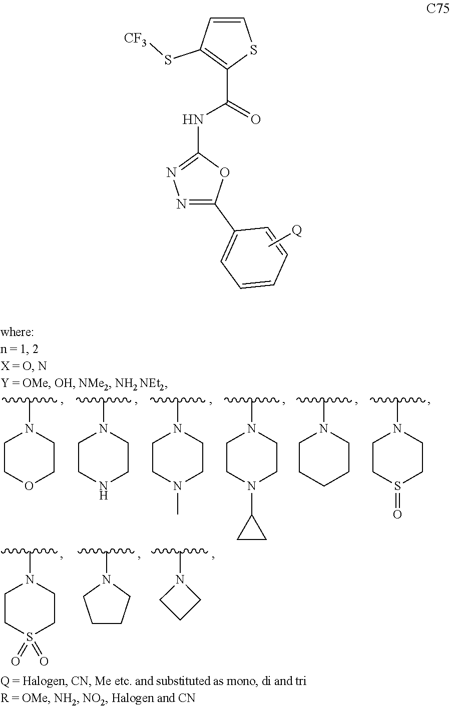

In some illustrative embodiments, the present invention relates to a compound having a formula (I) or (II), wherein the compound is

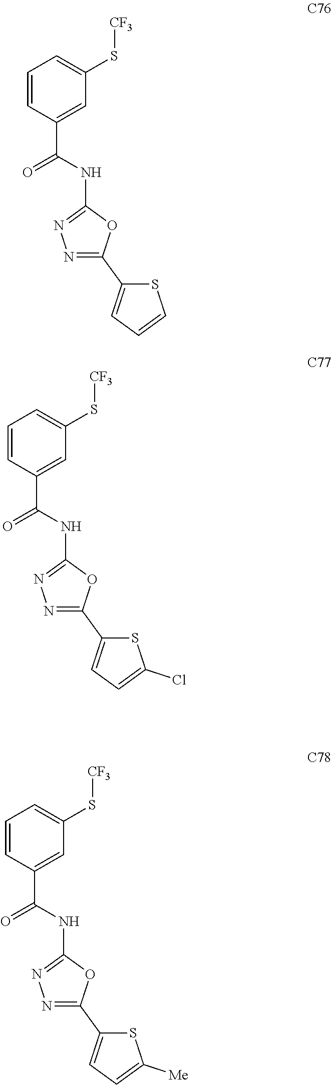 [SMILES: O=C(Nc1nnc(-c2cccs2)o1)c1cccc(SC(F)(F)F)c1 ; O=C(Nc1nnc(-c2ccc(Cl)s2)o1)c1cccc(SC(F)(F)F)c1 ; Cc1ccc(-c2nnc(NC(=O)c3cccc(SC(F)(F)F)c3)o2)s1]

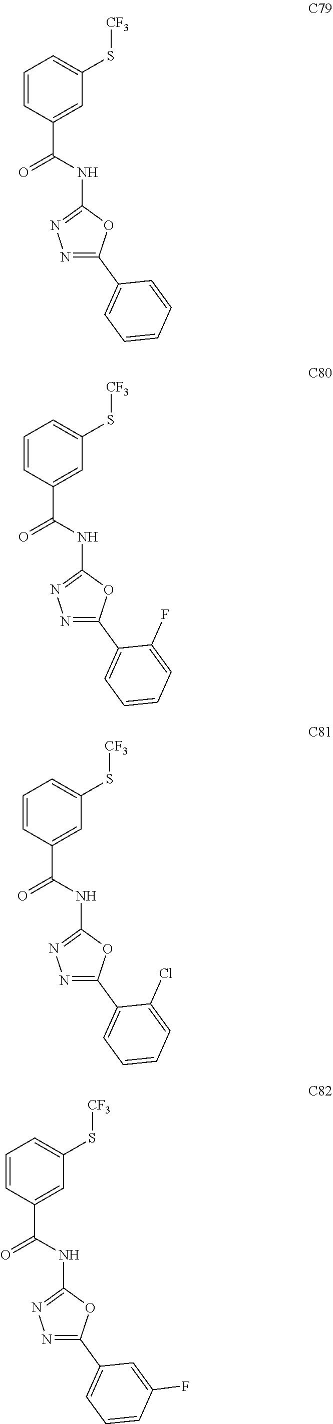

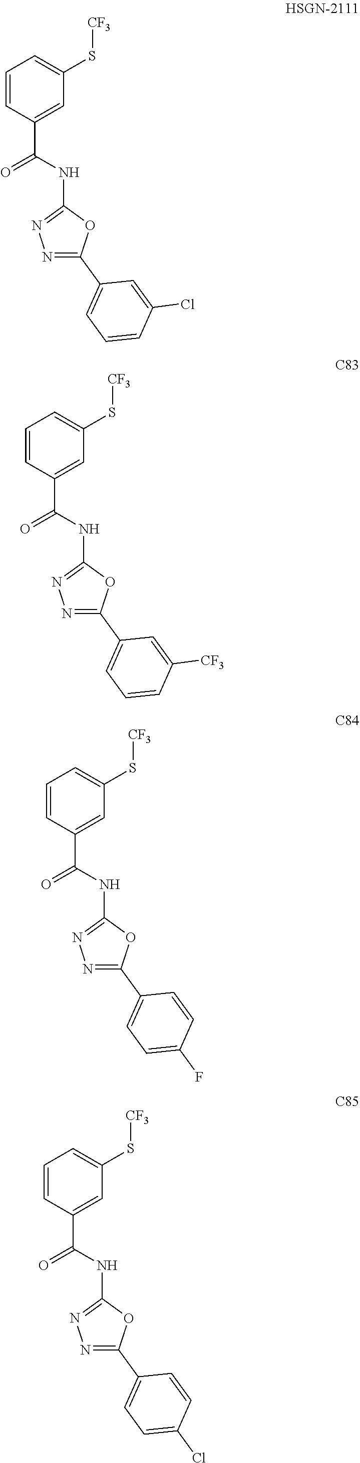

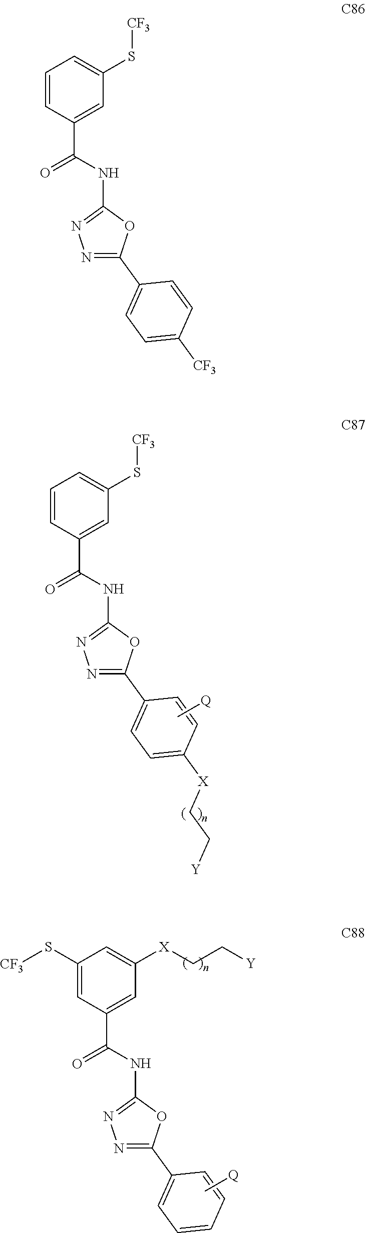

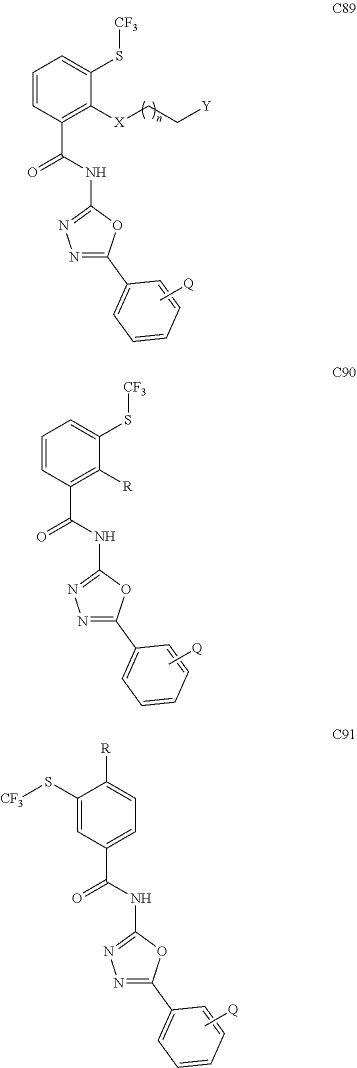

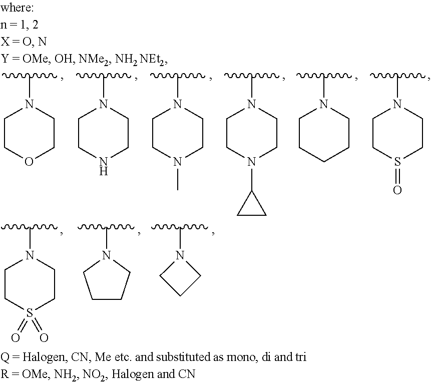

In some illustrative embodiments, the present invention relates to a compound having a formula (I) or (II), wherein the compound is

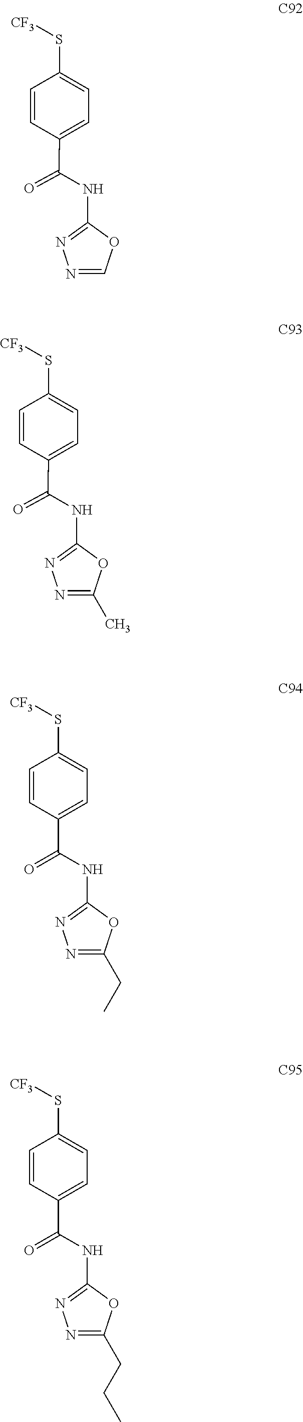 [SMILES: O=C(Nc1nnco1)c1ccc(SC(F)(F)F)cc1 ; Cc1nnc(NC(=O)c2ccc(SC(F)(F)F)cc2)o1 ; CCc1nnc(NC(=O)c2ccc(SC(F)(F)F)cc2)o1 ; CCCc1nnc(NC(=O)c2ccc(SC(F)(F)F)cc2)o1]

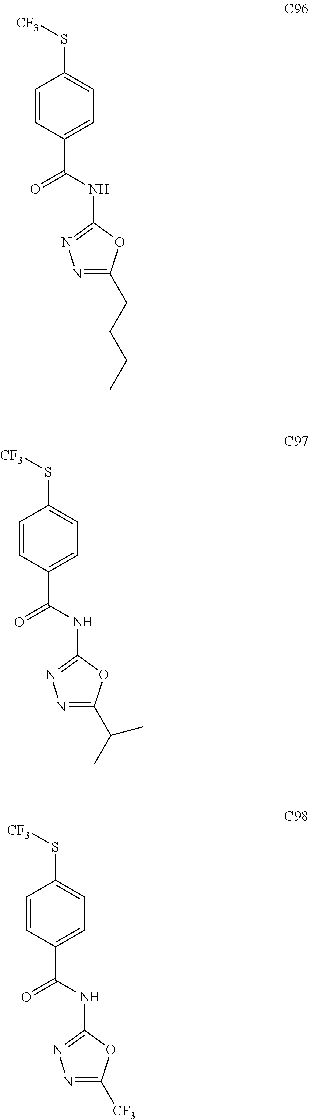 [SMILES: CCCCc1nnc(NC(=O)c2ccc(SC(F)(F)F)cc2)o1 ; CC(C)c1nnc(NC(=O)c2ccc(SC(F)(F)F)cc2)o1 ; O=C(Nc1nnc(C(F)(F)F)o1)c1ccc(SC(F)(F)F)cc1]

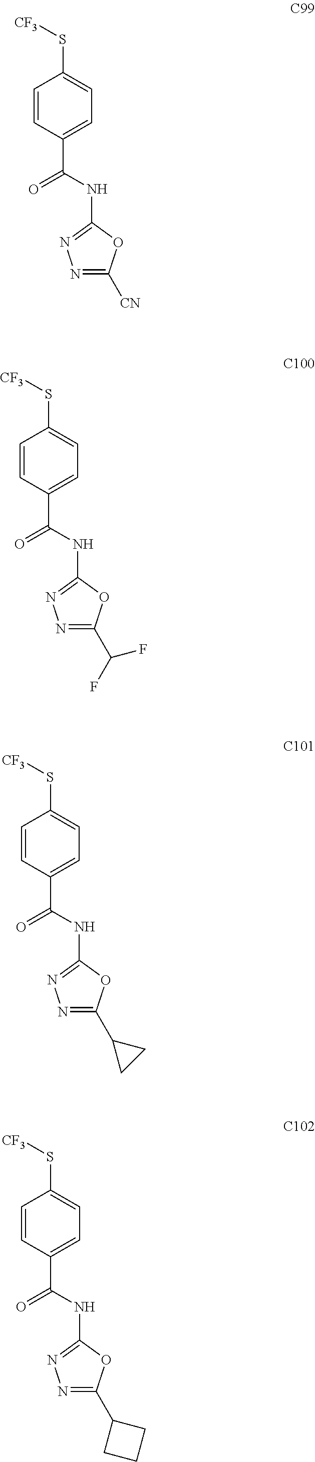 [SMILES: N#Cc1nnc(NC(=O)c2ccc(SC(F)(F)F)cc2)o1 ; O=C(Nc1nnc(C(F)F)o1)c1ccc(SC(F)(F)F)cc1 ; O=C(Nc1nnc(C2CC2)o1)c1ccc(SC(F)(F)F)cc1 ; O=C(Nc1nnc(C2CCC2)o1)c1ccc(SC(F)(F)F)cc1]

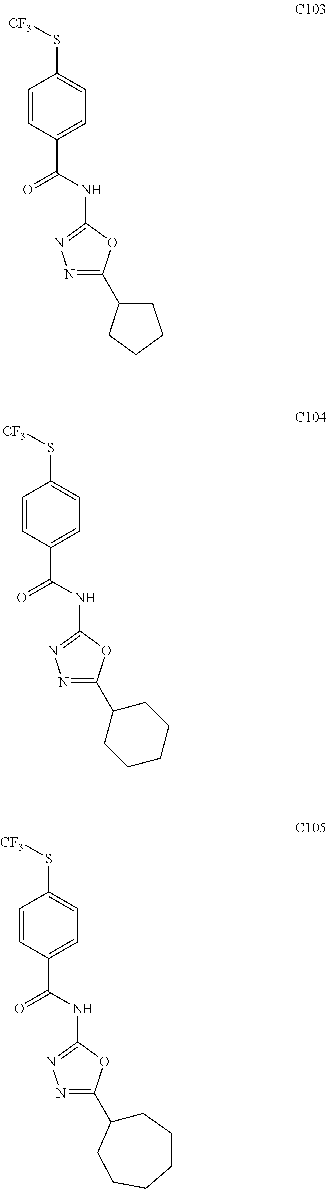 [SMILES: O=C(Nc1nnc(C2CCCC2)o1)c1ccc(SC(F)(F)F)cc1 ; O=C(Nc1nnc(C2CCCCC2)o1)c1ccc(SC(F)(F)F)cc1 ; O=C(Nc1nnc(C2CCCCCC2)o1)c1ccc(SC(F)(F)F)cc1]

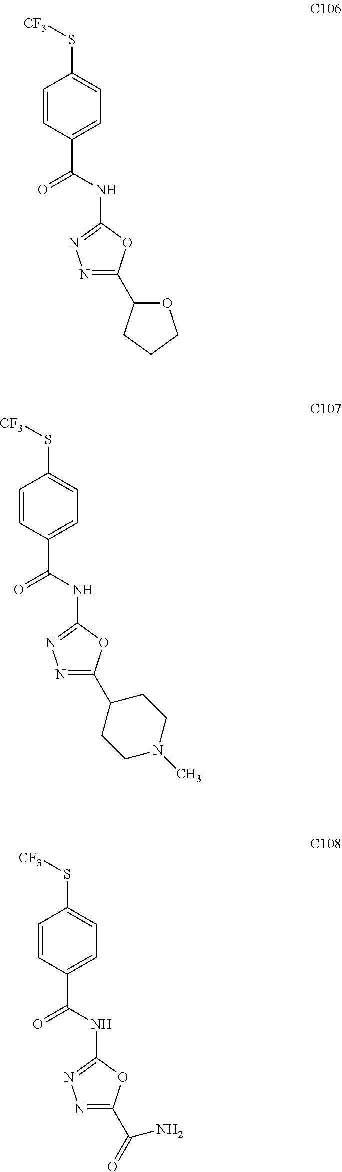 [SMILES: O=C(Nc1nnc(C2CCCO2)o1)c1ccc(SC(F)(F)F)cc1 ; CN1CCC(c2nnc(NC(=O)c3ccc(SC(F)(F)F)cc3)o2)CC1 ; NC(=O)c1nnc(NC(=O)c2ccc(SC(F)(F)F)cc2)o1]

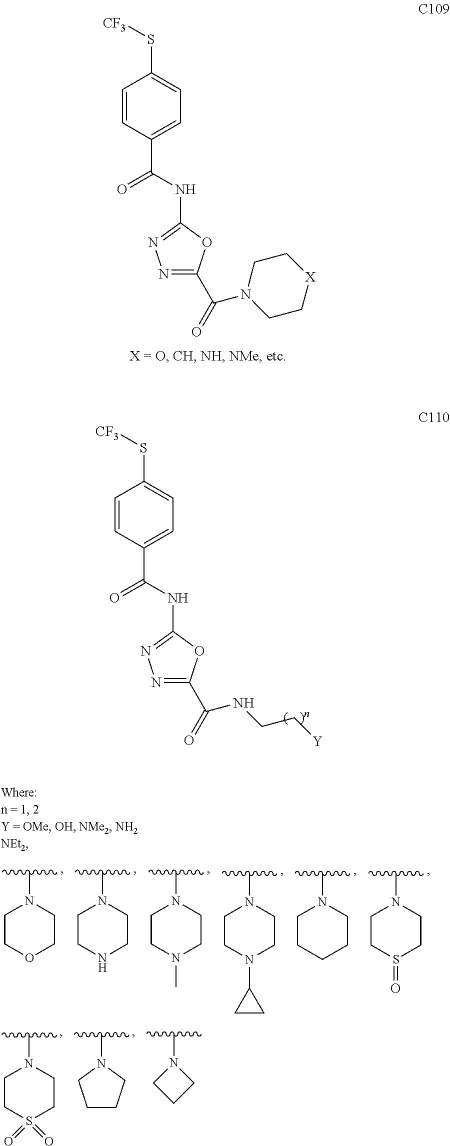

In some illustrative embodiments, the present invention relates to a compound having a formula (I) or (II), wherein the compound is

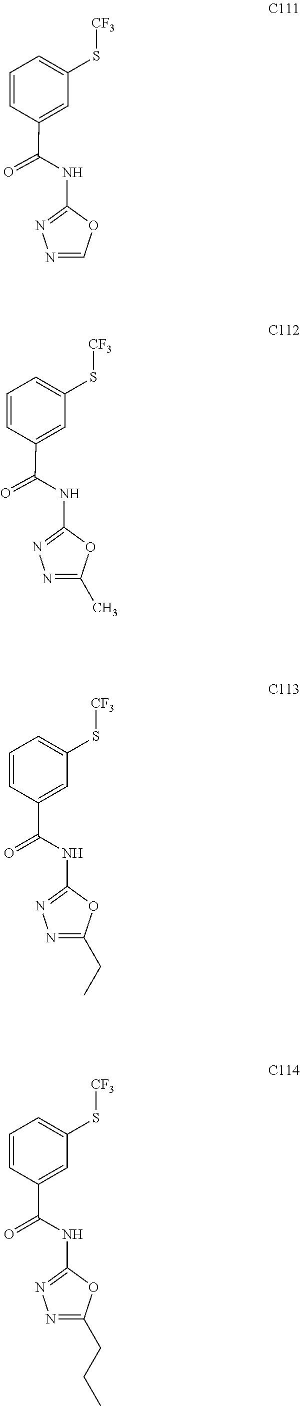 [SMILES: O=C(Nc1nnco1)c1cccc(SC(F)(F)F)c1 ; Cc1nnc(NC(=O)c2cccc(SC(F)(F)F)c2)o1 ; CCc1nnc(NC(=O)c2cccc(SC(F)(F)F)c2)o1 ; CCCc1nnc(NC(=O)c2cccc(SC(F)(F)F)c2)o1]

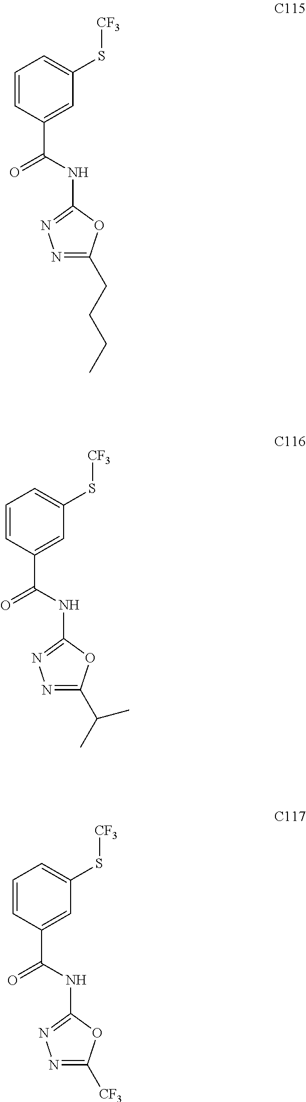 [SMILES: CCCCc1nnc(NC(=O)c2cccc(SC(F)(F)F)c2)o1 ; CC(C)c1nnc(NC(=O)c2cccc(SC(F)(F)F)c2)o1 ; O=C(Nc1nnc(C(F)(F)F)o1)c1cccc(SC(F)(F)F)c1]

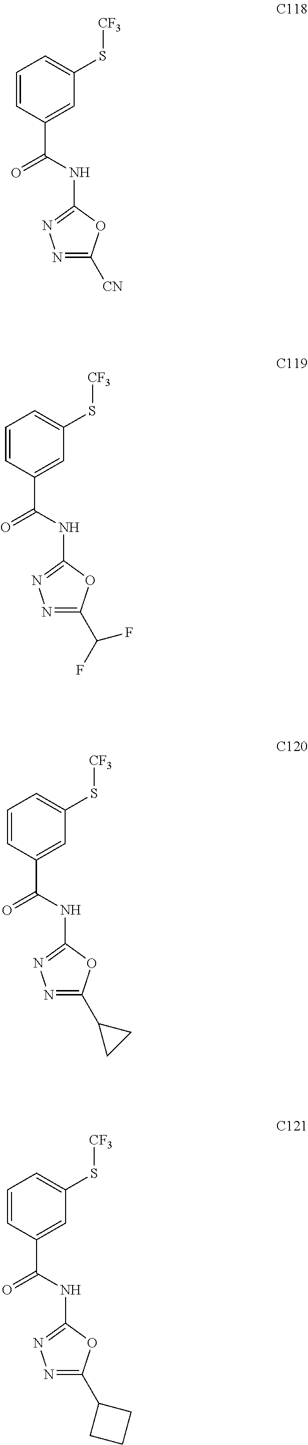 [SMILES: N#Cc1nnc(NC(=O)c2cccc(SC(F)(F)F)c2)o1 ; O=C(Nc1nnc(C(F)F)o1)c1cccc(SC(F)(F)F)c1 ; O=C(Nc1nnc(C2CC2)o1)c1cccc(SC(F)(F)F)c1 ; O=C(Nc1nnc(C2CCC2)o1)c1cccc(SC(F)(F)F)c1]

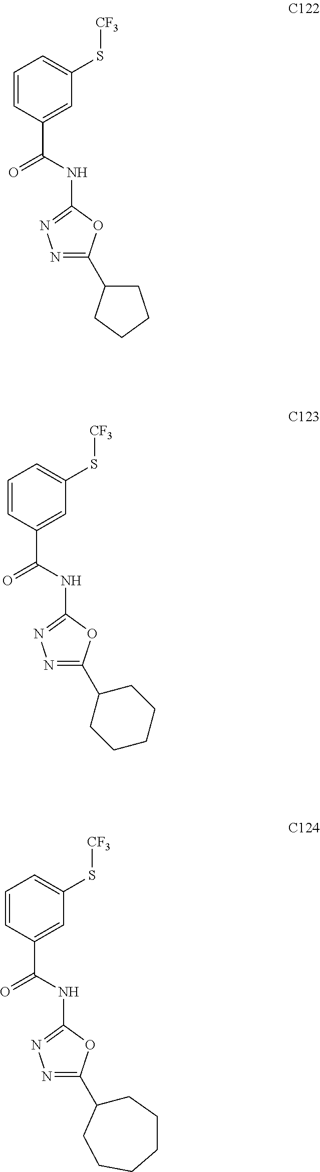 [SMILES: O=C(Nc1nnc(C2CCCC2)o1)c1cccc(SC(F)(F)F)c1 ; O=C(Nc1nnc(C2CCCCC2)o1)c1cccc(SC(F)(F)F)c1 ; O=C(Nc1nnc(C2CCCCCC2)o1)c1cccc(SC(F)(F)F)c1]

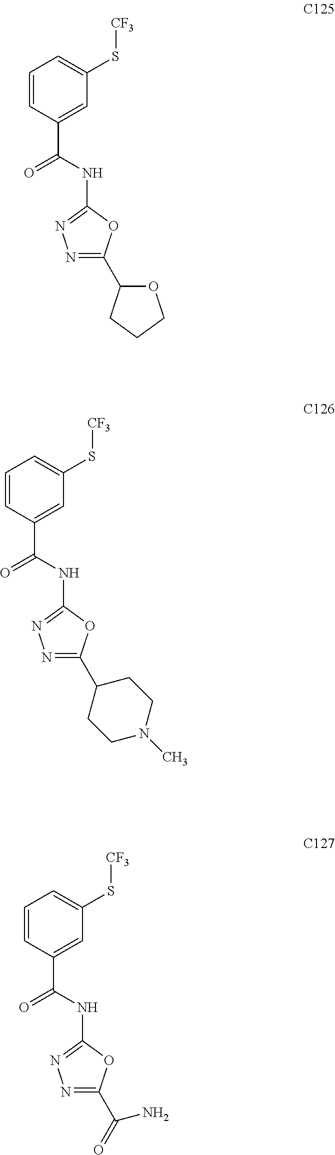 [SMILES: O=C(Nc1nnc(C2CCCO2)o1)c1cccc(SC(F)(F)F)c1 ; CN1CCC(c2nnc(NC(=O)c3cccc(SC(F)(F)F)c3)o2)CC1 ; NC(=O)c1nnc(NC(=O)c2cccc(SC(F)(F)F)c2)o1]

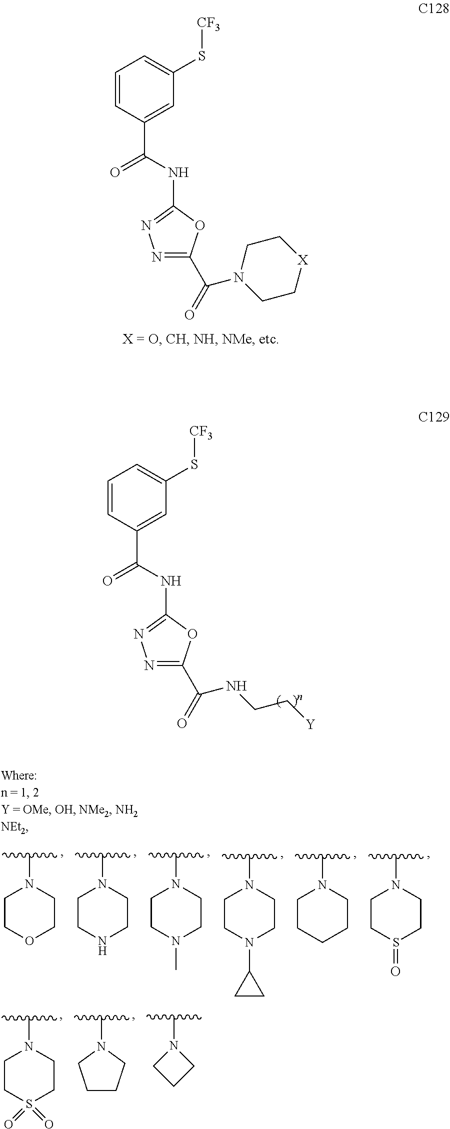

In some illustrative embodiments, the present invention relates to a compound having a formula (I) or (II), wherein the compound is

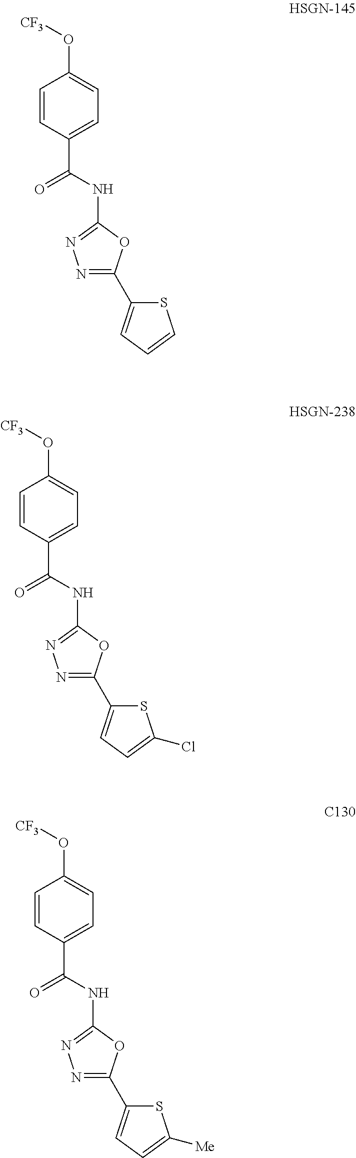 [SMILES: O=C(Nc1nnc(-c2cccs2)o1)c1ccc(OC(F)(F)F)cc1 ; O=C(Nc1nnc(-c2ccc(Cl)s2)o1)c1ccc(OC(F)(F)F)cc1 ; Cc1ccc(-c2nnc(NC(=O)c3ccc(OC(F)(F)F)cc3)o2)s1]

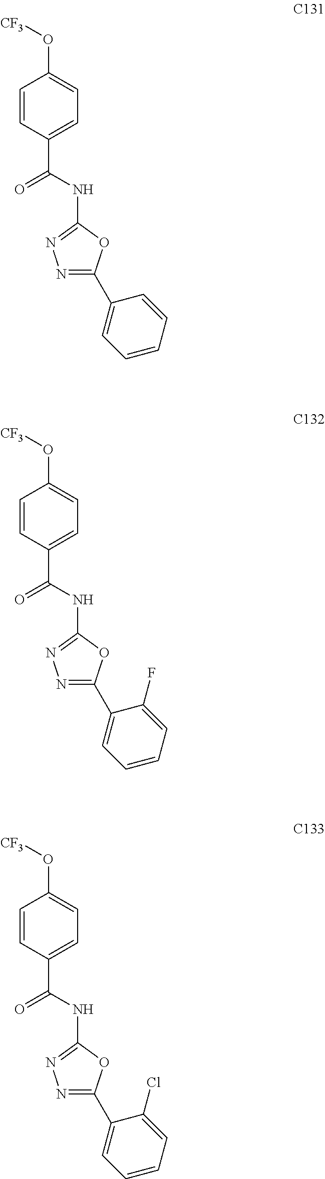 [SMILES: O=C(Nc1nnc(-c2ccccc2)o1)c1ccc(OC(F)(F)F)cc1 ; O=C(Nc1nnc(-c2ccccc2F)o1)c1ccc(OC(F)(F)F)cc1 ; O=C(Nc1nnc(-c2ccccc2Cl)o1)c1ccc(OC(F)(F)F)cc1]

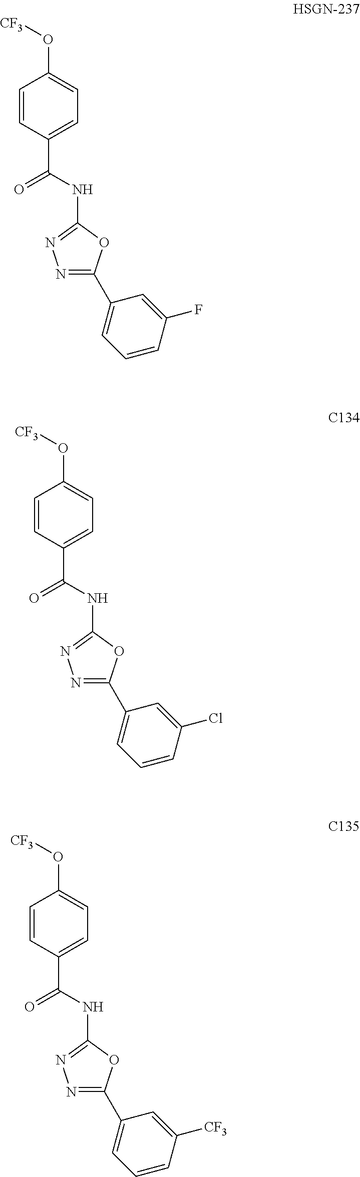 [SMILES: O=C(Nc1nnc(-c2cccc(F)c2)o1)c1ccc(OC(F)(F)F)cc1 ; O=C(Nc1nnc(-c2cccc(Cl)c2)o1)c1ccc(OC(F)(F)F)cc1 ; O=C(Nc1nnc(-c2cccc(C(F)(F)F)c2)o1)c1ccc(OC(F)(F)F)cc1]

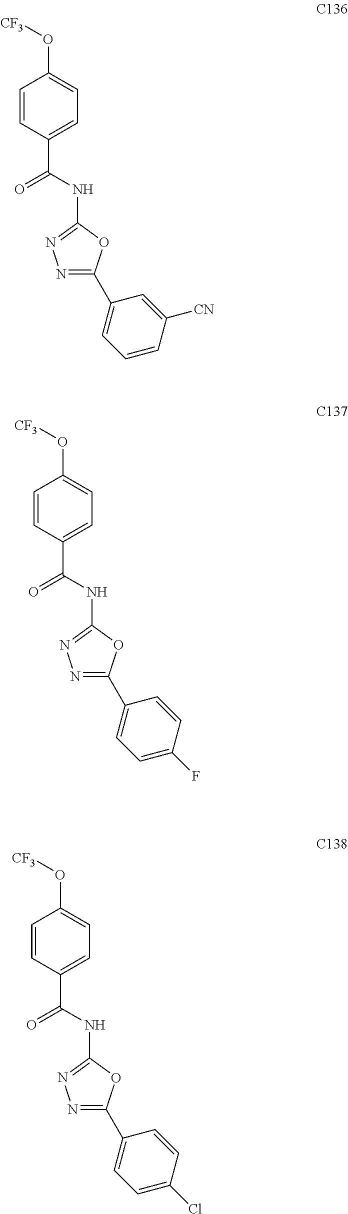 [SMILES: N#Cc1cccc(-c2nnc(NC(=O)c3ccc(OC(F)(F)F)cc3)o2)c1 ; O=C(Nc1nnc(-c2ccc(F)cc2)o1)c1ccc(OC(F)(F)F)cc1 ; O=C(Nc1nnc(-c2ccc(Cl)cc2)o1)c1ccc(OC(F)(F)F)cc1]

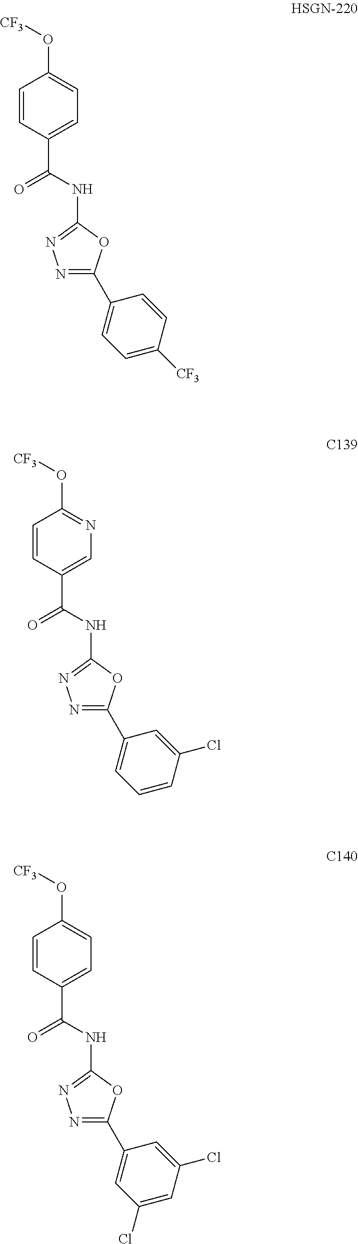 [SMILES: O=C(Nc1nnc(-c2ccc(C(F)(F)F)cc2)o1)c1ccc(OC(F)(F)F)cc1 ; O=C(Nc1nnc(-c2cccc(Cl)c2)o1)c1ccc(OC(F)(F)F)nc1 ; O=C(Nc1nnc(-c2cc(Cl)cc(Cl)c2)o1)c1ccc(OC(F)(F)F)cc1]

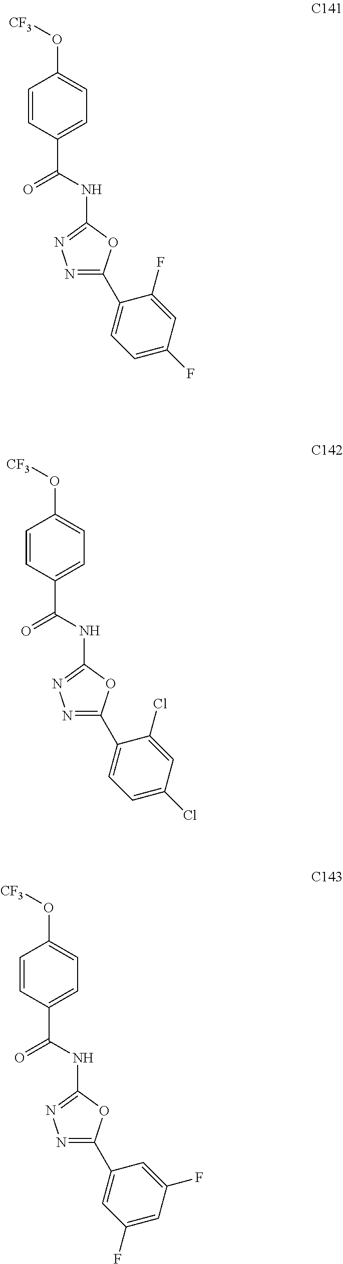 [SMILES: O=C(Nc1nnc(-c2ccc(F)cc2F)o1)c1ccc(OC(F)(F)F)cc1 ; O=C(Nc1nnc(-c2ccc(Cl)cc2Cl)o1)c1ccc(OC(F)(F)F)cc1 ; O=C(Nc1nnc(-c2cc(F)cc(F)c2)o1)c1ccc(OC(F)(F)F)cc1]

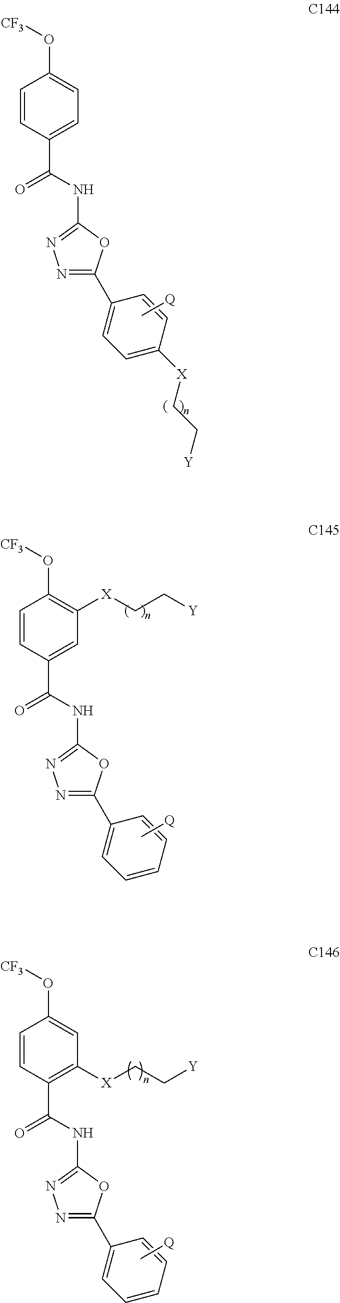

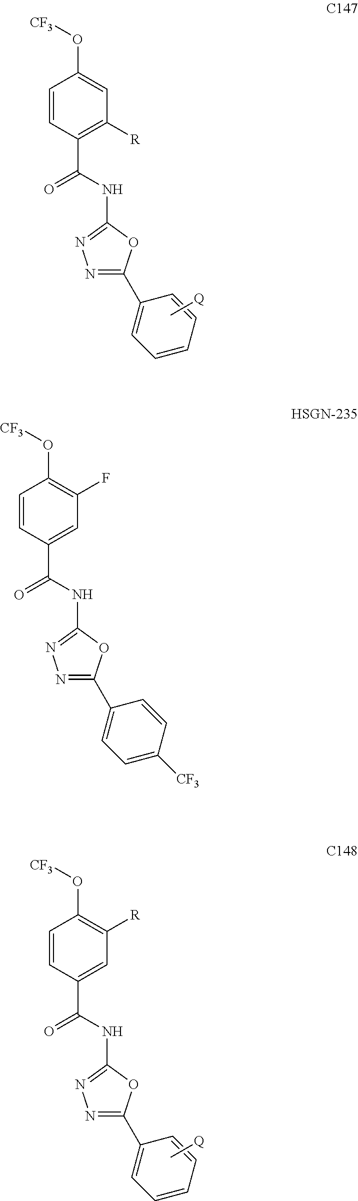

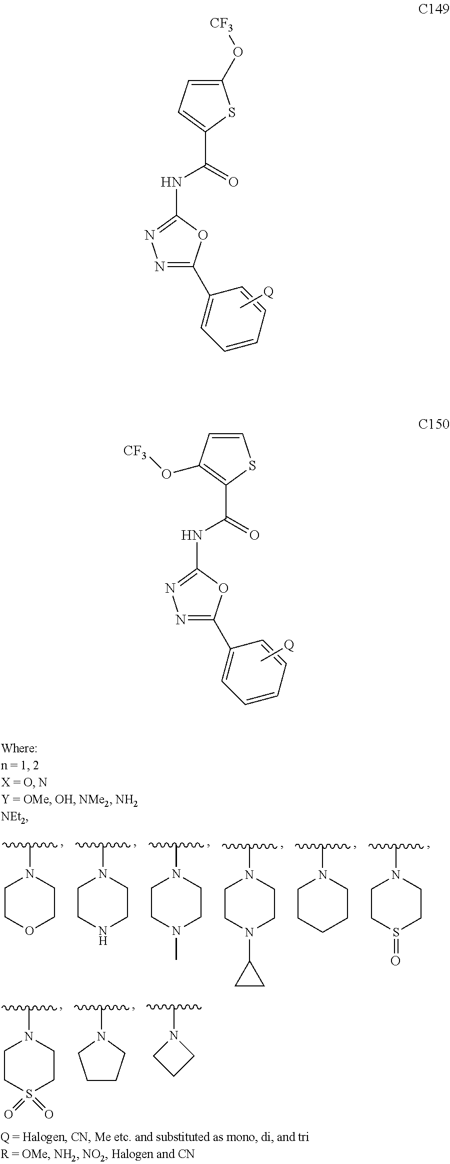

In some illustrative embodiments, the present invention relates to a compound having a formula (I) or (II), wherein the compound is

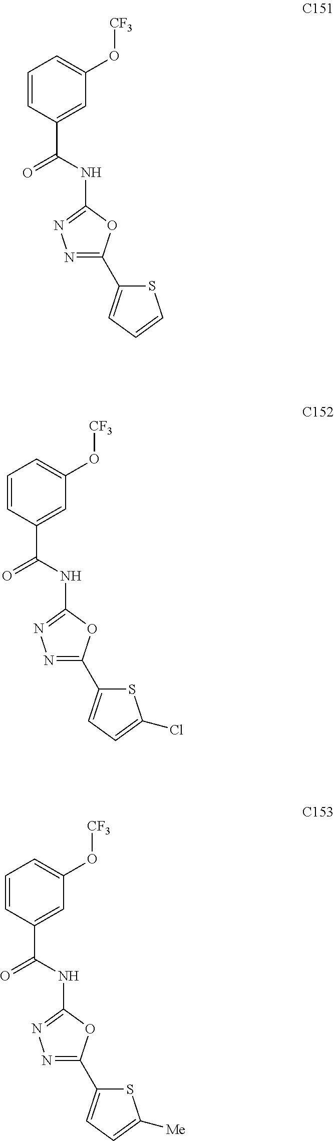 [SMILES: O=C(Nc1nnc(-c2cccs2)o1)c1cccc(OC(F)(F)F)c1 ; O=C(Nc1nnc(-c2ccc(Cl)s2)o1)c1cccc(OC(F)(F)F)c1 ; Cc1ccc(-c2nnc(NC(=O)c3cccc(OC(F)(F)F)c3)o2)s1]

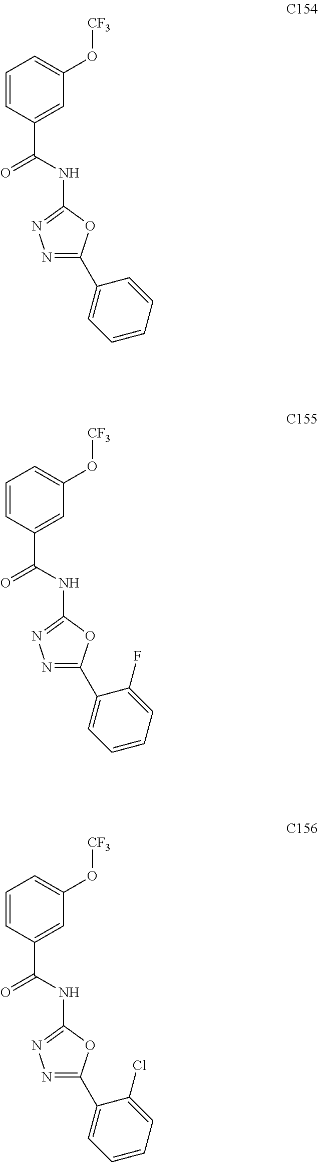 [SMILES: O=C(Nc1nnc(-c2ccccc2)o1)c1cccc(OC(F)(F)F)c1 ; O=C(Nc1nnc(-c2ccccc2F)o1)c1cccc(OC(F)(F)F)c1 ; O=C(Nc1nnc(-c2ccccc2Cl)o1)c1cccc(OC(F)(F)F)c1]

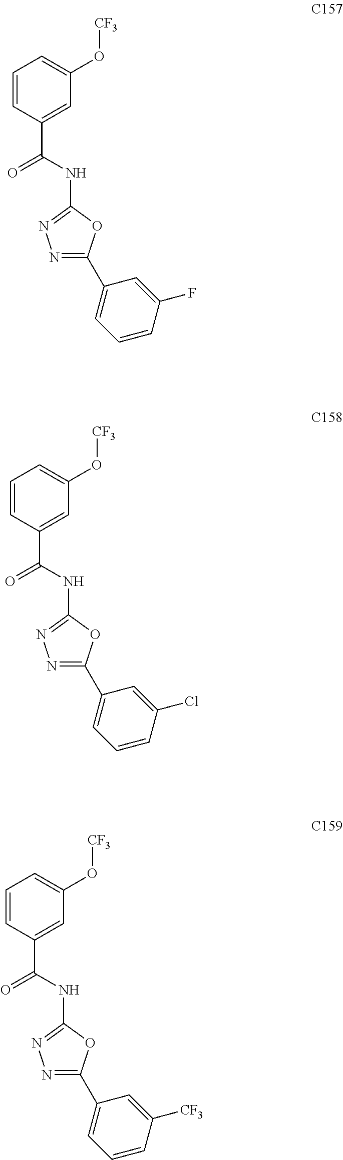 [SMILES: O=C(Nc1nnc(-c2cccc(F)c2)o1)c1cccc(OC(F)(F)F)c1 ; O=C(Nc1nnc(-c2cccc(Cl)c2)o1)c1cccc(OC(F)(F)F)c1 ; O=C(Nc1nnc(-c2cccc(C(F)(F)F)c2)o1)c1cccc(OC(F)(F)F)c1]

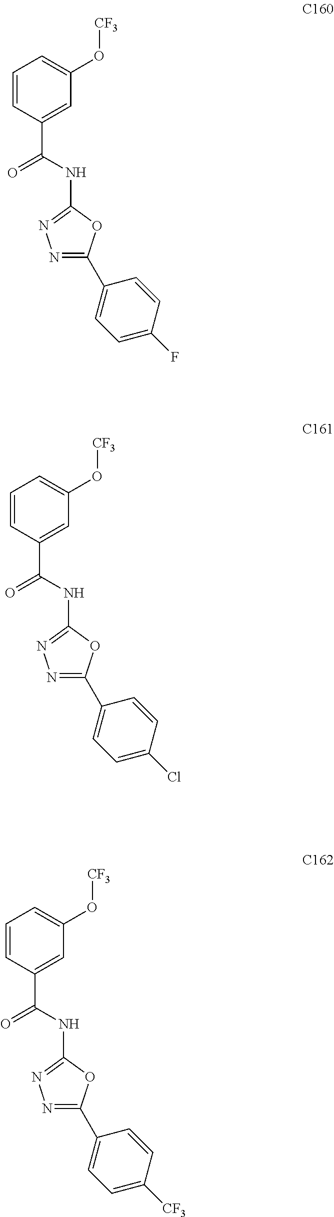 [SMILES: O=C(Nc1nnc(-c2ccc(F)cc2)o1)c1cccc(OC(F)(F)F)c1 ; O=C(Nc1nnc(-c2ccc(Cl)cc2)o1)c1cccc(OC(F)(F)F)c1 ; O=C(Nc1nnc(-c2ccc(C(F)(F)F)cc2)o1)c1cccc(OC(F)(F)F)c1]

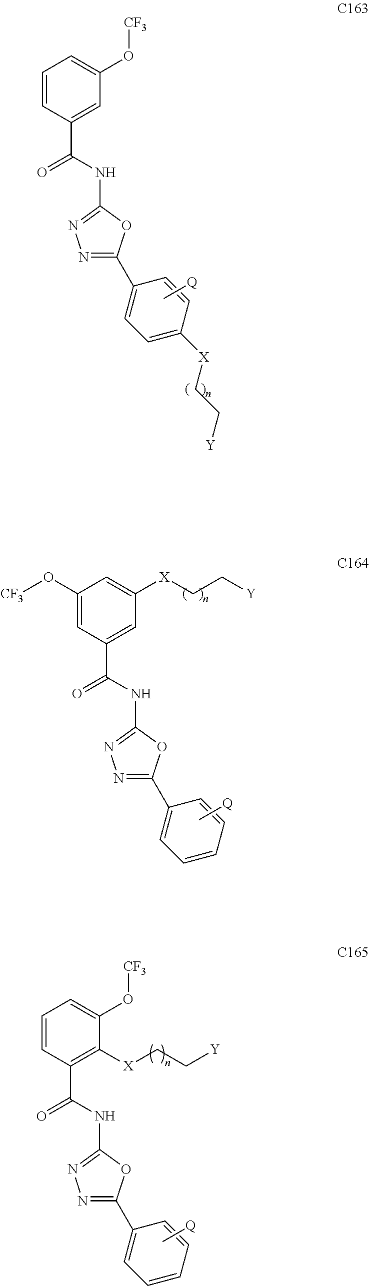

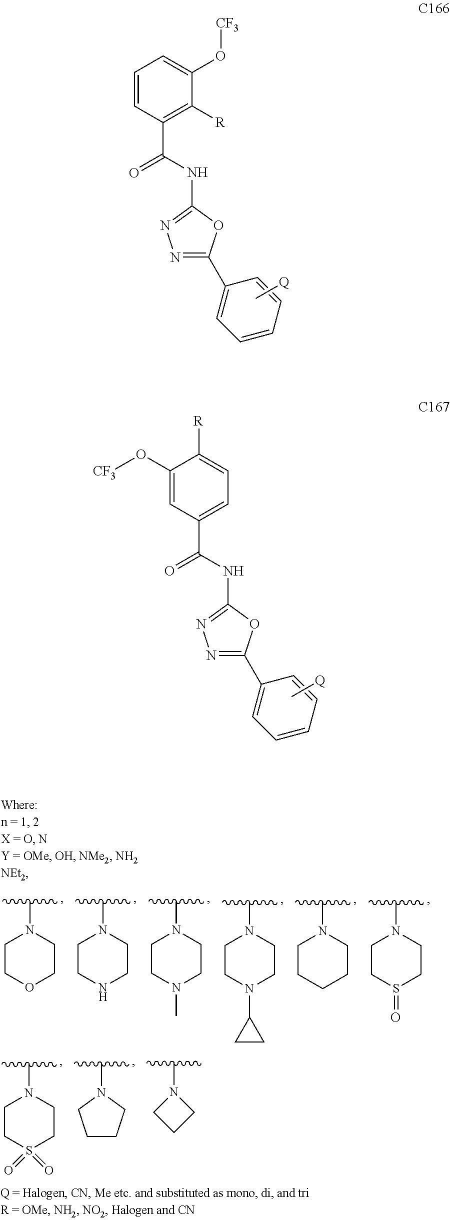

In some illustrative embodiments, the present invention relates to a compound having a formula (I) or (II), wherein the compound is

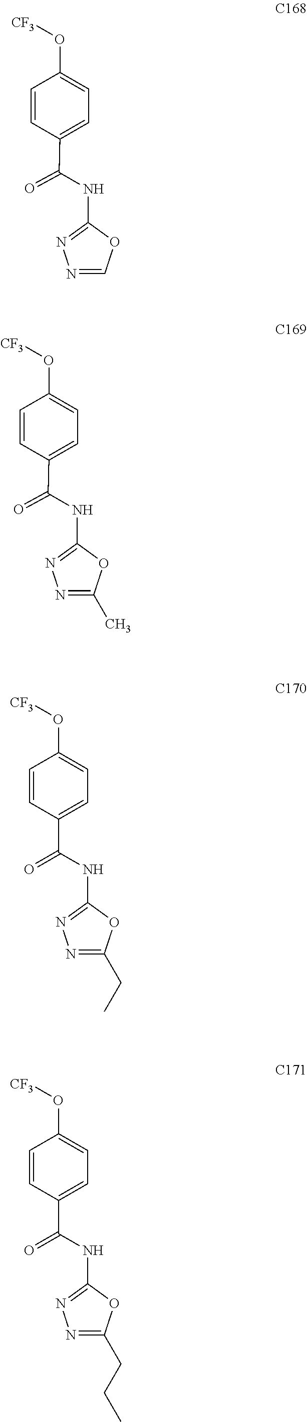 [SMILES: O=C(Nc1nnco1)c1ccc(OC(F)(F)F)cc1 ; Cc1nnc(NC(=O)c2ccc(OC(F)(F)F)cc2)o1 ; CCc1nnc(NC(=O)c2ccc(OC(F)(F)F)cc2)o1 ; CCCc1nnc(NC(=O)c2ccc(OC(F)(F)F)cc2)o1]

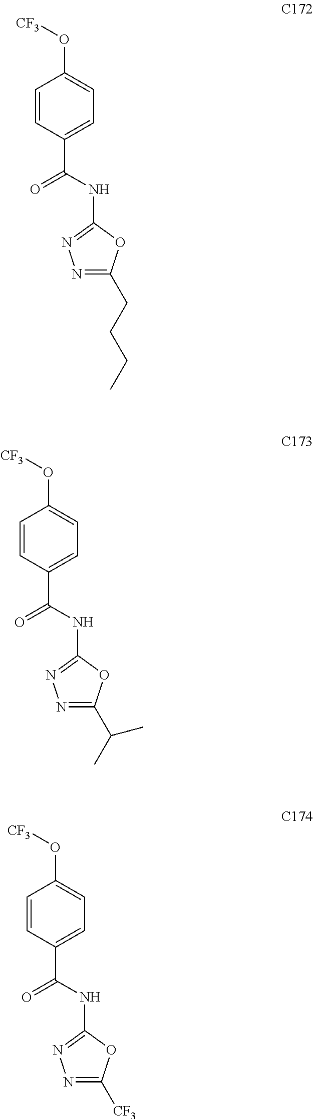 [SMILES: CCCCc1nnc(NC(=O)c2ccc(OC(F)(F)F)cc2)o1 ; CC(C)c1nnc(NC(=O)c2ccc(OC(F)(F)F)cc2)o1 ; O=C(Nc1nnc(C(F)(F)F)o1)c1ccc(OC(F)(F)F)cc1]

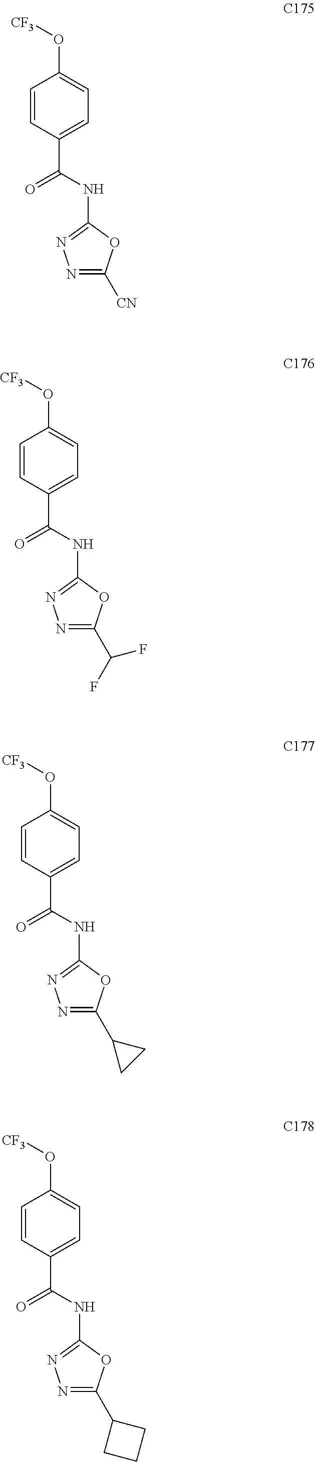 [SMILES: N#Cc1nnc(NC(=O)c2ccc(OC(F)(F)F)cc2)o1 ; O=C(Nc1nnc(C(F)F)o1)c1ccc(OC(F)(F)F)cc1 ; O=C(Nc1nnc(C2CC2)o1)c1ccc(OC(F)(F)F)cc1 ; O=C(Nc1nnc(C2CCC2)o1)c1ccc(OC(F)(F)F)cc1]

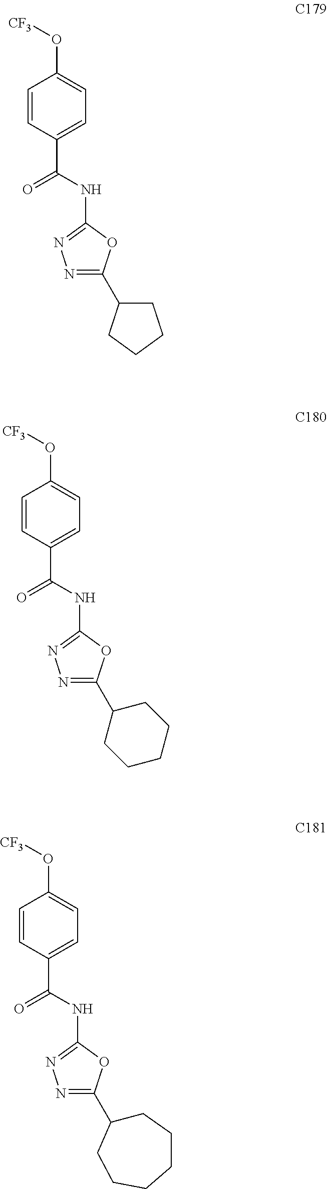 [SMILES: O=C(Nc1nnc(C2CCCC2)o1)c1ccc(OC(F)(F)F)cc1 ; O=C(Nc1nnc(C2CCCCC2)o1)c1ccc(OC(F)(F)F)cc1 ; O=C(Nc1nnc(C2CCCCCC2)o1)c1ccc(OC(F)(F)F)cc1]

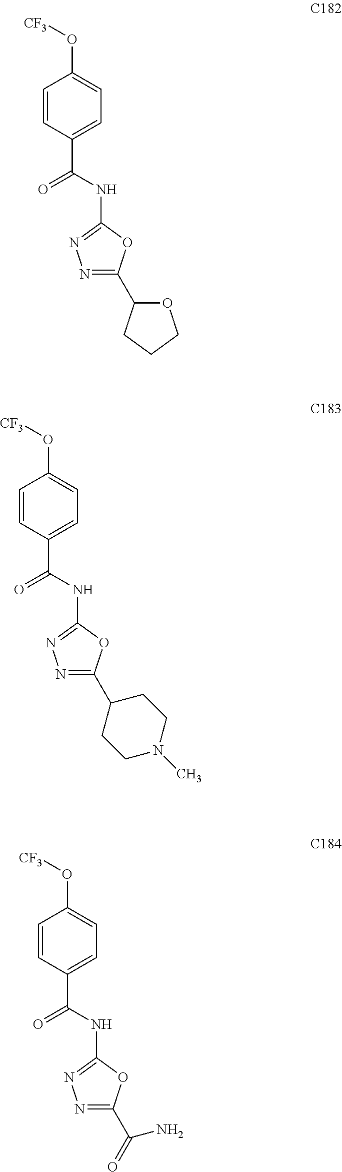 [SMILES: O=C(Nc1nnc(C2CCCO2)o1)c1ccc(OC(F)(F)F)cc1 ; CN1CCC(c2nnc(NC(=O)c3ccc(OC(F)(F)F)cc3)o2)CC1 ; NC(=O)c1nnc(NC(=O)c2ccc(OC(F)(F)F)cc2)o1]

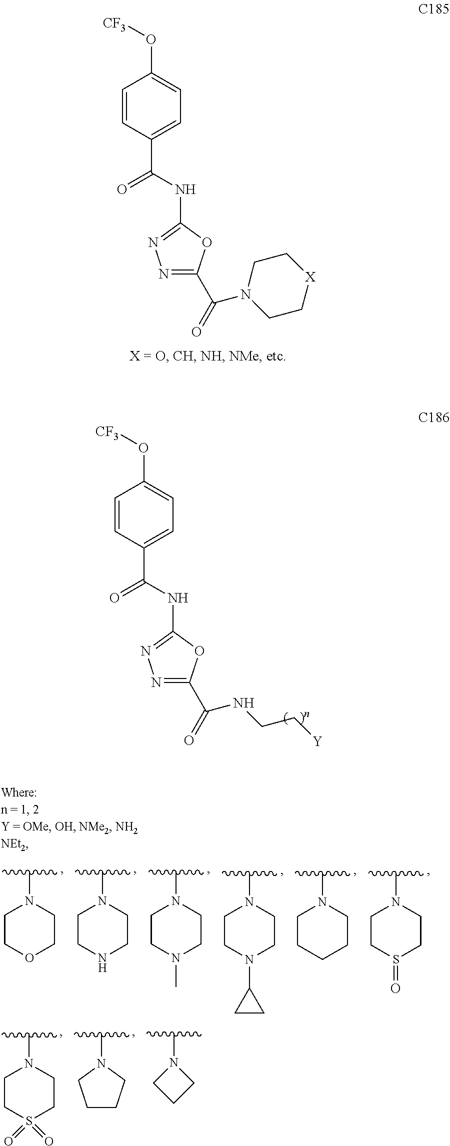

In some illustrative embodiments, the present invention relates to a compound having a formula (I) or (II), wherein the compound is

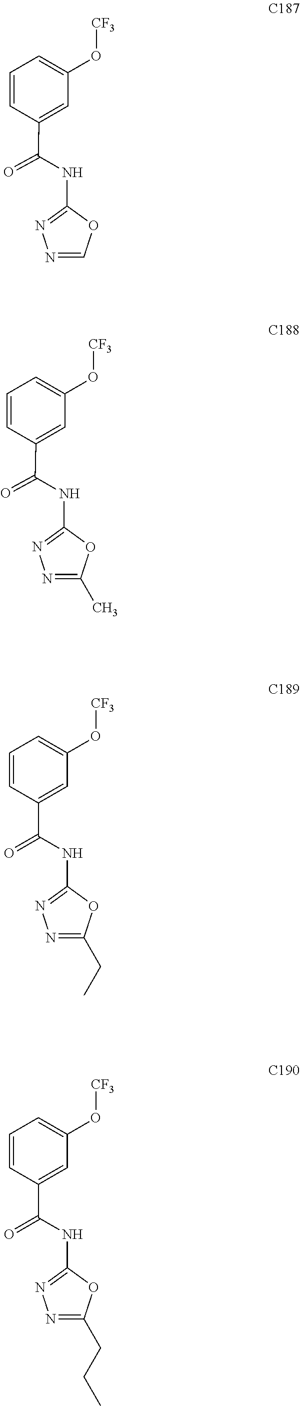 [SMILES: O=C(Nc1nnco1)c1cccc(OC(F)(F)F)c1 ; Cc1nnc(NC(=O)c2cccc(OC(F)(F)F)c2)o1 ; CCc1nnc(NC(=O)c2cccc(OC(F)(F)F)c2)o1 ; CCCc1nnc(NC(=O)c2cccc(OC(F)(F)F)c2)o1]

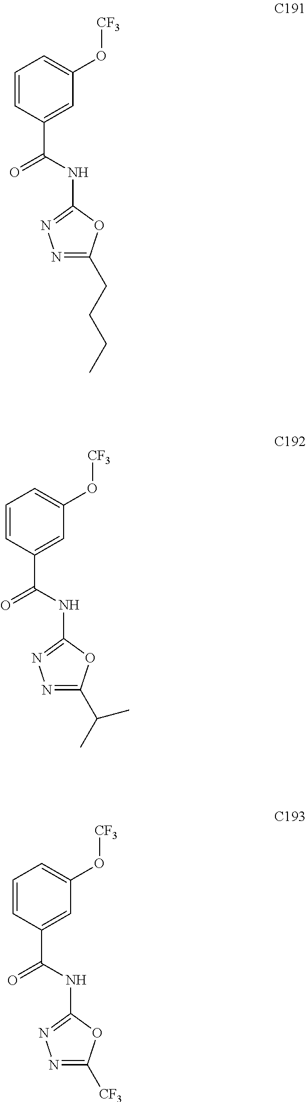 [SMILES: CCCCc1nnc(NC(=O)c2cccc(OC(F)(F)F)c2)o1 ; CC(C)c1nnc(NC(=O)c2cccc(OC(F)(F)F)c2)o1 ; O=C(Nc1nnc(C(F)(F)F)o1)c1cccc(OC(F)(F)F)c1]

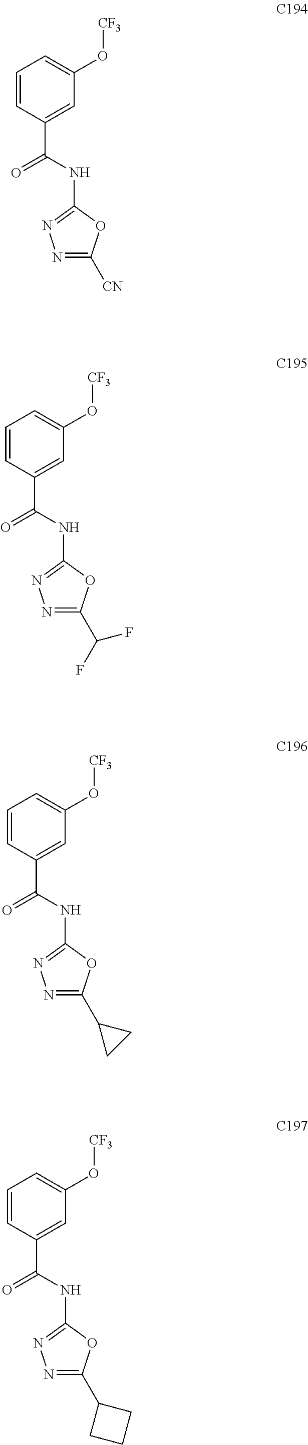 [SMILES: N#Cc1nnc(NC(=O)c2cccc(OC(F)(F)F)c2)o1 ; O=C(Nc1nnc(C(F)F)o1)c1cccc(OC(F)(F)F)c1 ; O=C(Nc1nnc(C2CC2)o1)c1cccc(OC(F)(F)F)c1 ; O=C(Nc1nnc(C2CCC2)o1)c1cccc(OC(F)(F)F)c1]

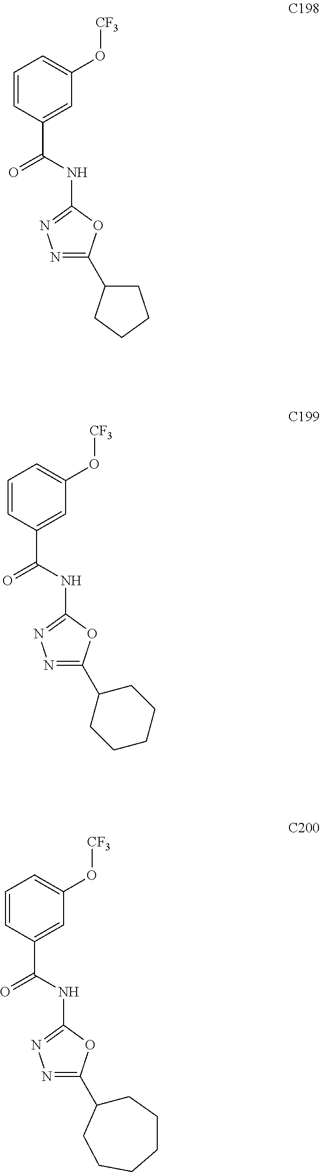 [SMILES: O=C(Nc1nnc(C2CCCC2)o1)c1cccc(OC(F)(F)F)c1 ; O=C(Nc1nnc(C2CCCCC2)o1)c1cccc(OC(F)(F)F)c1 ; O=C(Nc1nnc(C2CCCCCC2)o1)c1cccc(OC(F)(F)F)c1]

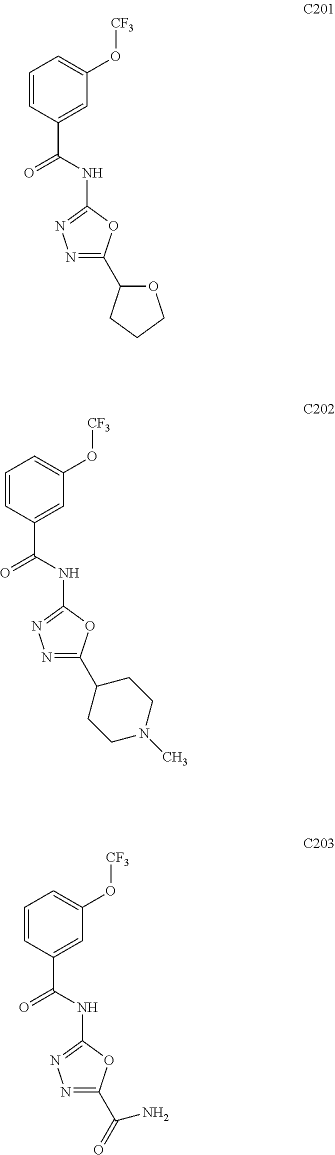 [SMILES: O=C(Nc1nnc(C2CCCO2)o1)c1cccc(OC(F)(F)F)c1 ; CN1CCC(c2nnc(NC(=O)c3cccc(OC(F)(F)F)c3)o2)CC1 ; NC(=O)c1nnc(NC(=O)c2cccc(OC(F)(F)F)c2)o1]

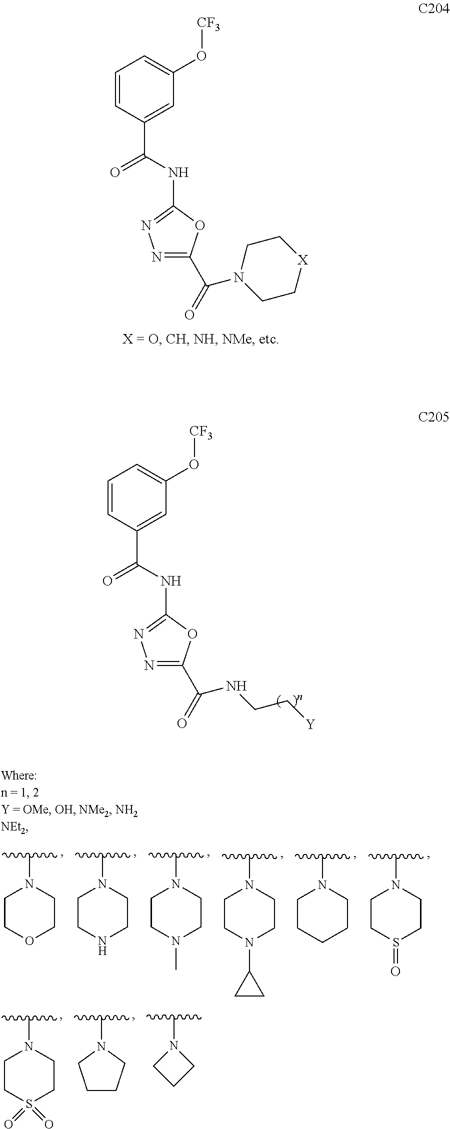

In some illustrative embodiments, the present invention relates to a compound having a formula (I) or (II), wherein the compound is

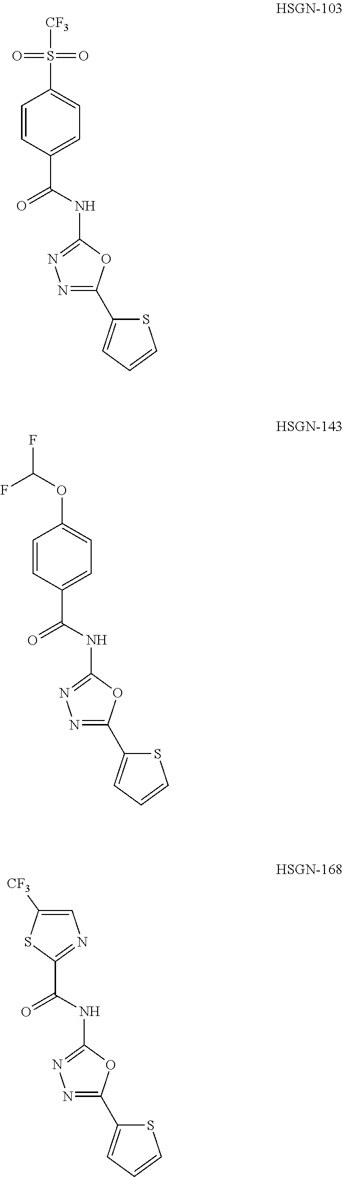 [SMILES: O=C(Nc1nnc(-c2cccs2)o1)c1ccc(S(=O)(=O)C(F)(F)F)cc1 ; O=C(Nc1nnc(-c2cccs2)o1)c1ccc(OC(F)F)cc1 ; O=C(Nc1nnc(-c2cccs2)o1)c1ncc(C(F)(F)F)s1]

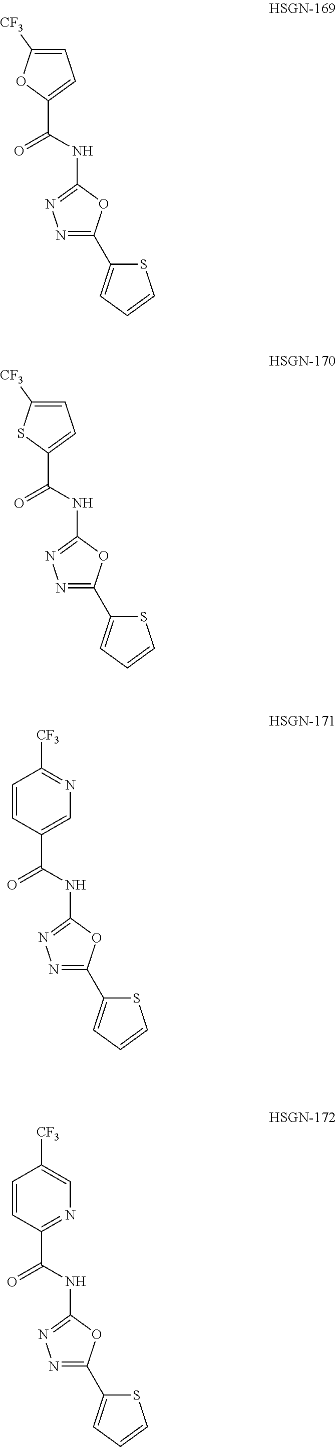 [SMILES: O=C(Nc1nnc(-c2cccs2)o1)c1ccc(C(F)(F)F)o1 ; O=C(Nc1nnc(-c2cccs2)o1)c1ccc(C(F)(F)F)s1 ; O=C(Nc1nnc(-c2cccs2)o1)c1ccc(C(F)(F)F)nc1 ; O=C(Nc1nnc(-c2cccs2)o1)c1ccc(C(F)(F)F)cn1]

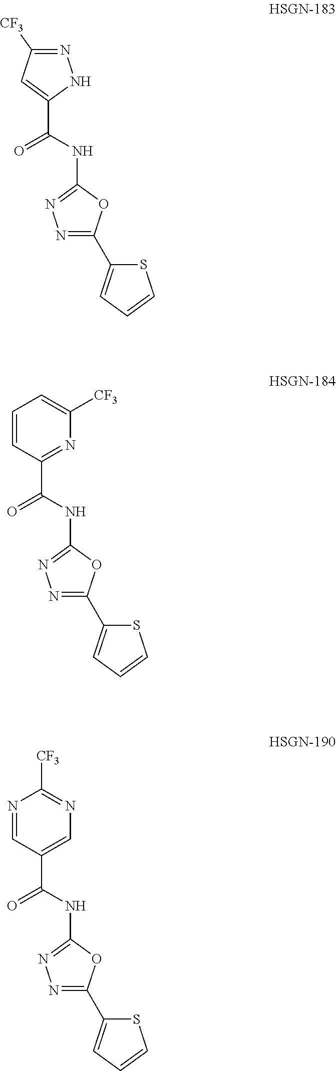 [SMILES: O=C(Nc1nnc(-c2cccs2)o1)c1cc(C(F)(F)F)n[nH]1 ; O=C(Nc1nnc(-c2cccs2)o1)c1cccc(C(F)(F)F)n1 ; O=C(Nc1nnc(-c2cccs2)o1)c1cnc(C(F)(F)F)nc1]

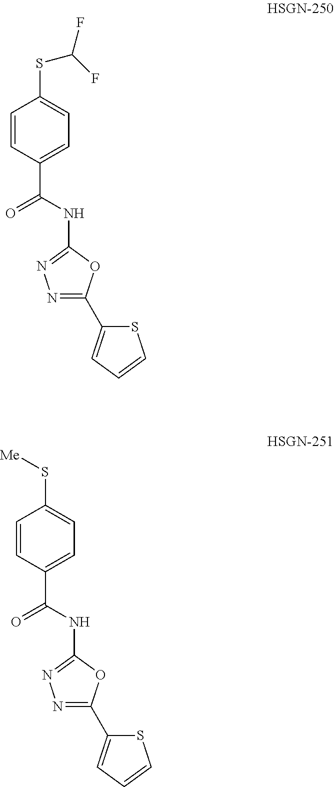 [SMILES: O=C(Nc1nnc(-c2cccs2)o1)c1ccc(SC(F)F)cc1 ; CSc1ccc(C(=O)Nc2nnc(-c3cccs3)o2)cc1]

In some illustrative embodiments, the present invention relates to a method for treating a patient with an infection comprising the step of administering a therapeutically effective amount of one or more compounds as disclosed herein, and one or more carriers, diluents, or excipients, to a patient in need of relief from said infection.

In some illustrative embodiments, the present invention relates to a method for treating a patient with an infection comprising the step of administering a therapeutically effective amount of one or more compounds as disclosed herein, in combination with one or more other compounds of the same or different mode of action, and one or more carriers, diluents, or excipients, to a patient in need of relief from said infection.

In some illustrative embodiments, the present invention relates to a method for treating a patient with an infection comprising the step of administering a therapeutically effective amount of one or more compounds as disclosed herein, and one or more carriers, diluents, or excipients, to a patient in need of relief from said infection, wherein the conjugate confers cell-type or tissue type targeting or the conjugate targets another pathway that synergizes the action of compounds disclosed herein.

In some illustrative embodiments, the present invention relates to a method for treating a patient with an infection comprising the step of administering a therapeutically effective amount of one or more compounds as disclosed herein, and one or more carriers, diluents, or excipients, to a patient in need of relief from said infection, wherein the conjugate confers an improved aqueous solubility or a low clearance.

In some illustrative embodiments, the present invention relates to a pharmaceutical composition comprising one or more compounds as disclosed herein, together with one or more pharmaceutically acceptable diluents, excipients or carriers.

In some illustrative embodiments, the present invention relates to a pharmaceutical composition comprising one or more compounds as disclosed herein, together with one or more pharmaceutically acceptable diluents, excipients or carriers, wherein said pharmaceutical composition is for the treatment of a bacterial infection.

In some illustrative embodiments, the present invention relates to the use of one or more compounds disclosed herein, together with one or more pharmaceutically acceptable diluents, excipients or carriers, in the manufacture of a medicament for the treatment of a bacterial infection.

In some illustrative embodiments, the present invention relates to a pharmaceutical composition comprising nanoparticles of one or more compounds disclosed herein, together with one or more diluents, excipients or carriers.

In some illustrative embodiments, the present invention relates to a prodrug comprising one or more compounds as disclosed herein, wherein the prodrug moiety is removed at specific location, such as gastrointestinal or in blood or in tissues or in cancer specific.

In some illustrative embodiments, the present invention relates to an analog of compounds disclosed herein, wherein specific metabolic hot spots are modified with groups such as deuterium or fluorine.

In some illustrative embodiments, the present invention relates to a method for treating an infection disease comprising the step of administering a therapeutically effective amount of one or more compounds as disclosed herein, and one or more carriers, diluents, or excipients, to a patient or animal in need of relief from said infection, wherein said infection is an infection caused by MRSA, VISA, VRSA, VRE, methicillin-resistant S. aureus, E. faecalis, VRE, E. faecium, S. pneumoniae, S. pseudopneumoniae, S. pyogenes, S. sanguinis, S. sobrinus, S. intermedius, S. anginosus, S. mitis, S. mutans, S. oralis, S. tigurinus, S. constellatus, S. bovis, L. monocytogenes, C. difficile, C. perfringens, C. tetani, C. botulinum, N gonorrhoeae, E. rhusiopathiae, B. anthracis, C. diphtheriae, S. suis, S. iniae, S. equi, S. dysgalactiae.

In some other embodiments, the present invention relates to a method for treating an infection disease comprising the step of administering a therapeutically effective amount of one or more compounds disclosed herein, and one or more carriers, diluents, or excipients, to a patient in need of relief from said infection.

In some other embodiments, the present invention relates to a method for treating an infection disease comprising the step of administering a therapeutically effective amount of a compound disclosed herein, in combination with one or more other compounds of the same or different mode of action, and one or more carriers, diluents, or excipients, to a patient in need of relief from said infection.

As used herein, the following terms and phrases shall have the meanings set forth below.

Unless defined otherwise, all technical and scientific terms used herein have the same meaning as commonly understood to one of ordinary skill in the art.

In the present disclosure the term “about” can allow for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, 99%, 99.5%, 99.9%, 99.99%, or at least about 99.999% or more of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

The term “substituted” as used herein refers to a functional group in which one or more hydrogen atoms contained therein are replaced by one or more non-hydrogen atoms. The term “functional group” or “substituent” as used herein refers to a group that can be or is substituted onto a molecule. Examples of substituents or functional groups include, but are not limited to, a halogen (e.g., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, azides, hydroxylamines, cyano, nitro groups, N-oxides, hydrazides, and enamines; and other heteroatoms in various other groups.

The term “alkyl” as used herein refers to substituted or unsubstituted straight chain and branched alkyl groups and cycloalkyl groups having from 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (Cₗ-C₆). Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to substituted or unsubstituted straight chain and branched divalent alkenyl and cycloalkenyl groups having from 2 to 20 carbon atoms(C₂-C₂₀), 2 to 12 carbons (C₂-C₁₂), 2 to 8 carbon atoms (C₂-C₈) or, in some embodiments, from 2 to 4 carbon atoms (C₂-C₄) and at least one carbon-carbon double bond. Examples of straight chain alkenyl groups include those with from 2 to 8 carbon atoms such as —CH═CH—, —CH═CHCH₂—, and the like. Examples of branched alkenyl groups include, but are not limited to, —CH═(CH₃)— and the like.

An alkynyl group is the fragment, containing an open point of attachment on a carbon atom that would form if a hydrogen atom bonded to a triply bonded carbon is removed from the molecule of an alkyne. The term “hydroxyalkyl” as used herein refers to alkyl groups as defined herein substituted with at least one hydroxyl (—OH) group.

The term “cycloalkyl” as used herein refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. In some embodiments, cycloalkyl groups can have 3 to 6 carbon atoms (C₃-C₆). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning here. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “aryl” as used herein refers to substituted or unsubstituted cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The term “aralkyl” and “arylalkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to substituted or unsubstituted aromatic and non-aromatic ring compounds containing 3 or more ring members, of which, one or more is a heteroatom such as, but not limited to, B, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆) or 6 to 8 carbon atoms (C₆-C₈).

A heteroaryl ring is an embodiment of a heterocyclyl group. The phrase “heterocyclyl group” includes fused ring species including those that include fused aromatic and non-aromatic groups. Representative heterocyclyl groups include, but are not limited to pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, and benzimidazolinyl groups.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclylalkyl groups include, but are not limited to, furan-2-yl methyl, furan-3-yl methyl, pyridine-3-yl methyl, tetrahydrofuran-2-yl methyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl,—CF(CH₃)₂ and the like.

The term “optionally substituted,” or “optional substituents,” as used herein, means that the groups in question are either unsubstituted or substituted with one or more of the substituents specified. When the groups in question are substituted with more than one substituent, the substituents may be the same or different. When using the terms “independently,” “independently are,” and “independently selected from” mean that the groups in question may be the same or different. Certain of the herein defined terms may occur more than once in the structure, and upon such occurrence each term shall be defined independently of the other.

The compounds described herein may contain one or more chiral centers, or may otherwise be capable of existing as multiple stereoisomers. It is to be understood that in one embodiment, the invention described herein is not limited to any particular stereochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. It is to be understood that in another embodiment, the invention described herein is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds, while including mixtures of geometry at one or more other double bonds.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

The term “prodrug” means a derivative of a compound that can hydrolyze, oxidize, or otherwise react under biological conditions (in vitro or in vivo) to provide an active compound, particularly a compound of the invention. Examples of prodrugs include, but are not limited to, derivatives and metabolites of a compound of the invention that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

Further, in each of the foregoing and following embodiments, it is to be understood that the formulae include and represent not only all pharmaceutically acceptable salts of the compounds, but also include any and all hydrates and/or solvates of the compound formulae or salts thereof. It is to be appreciated that certain functional groups, such as the hydroxy, amino, and like groups form complexes and/or coordination compounds with water and/or various solvents, in the various physical forms of the compounds. Accordingly, the above formulae are to be understood to include and represent those various hydrates and/or solvates. In each of the foregoing and following embodiments, it is also to be understood that the formulae include and represent each possible isomer, such as stereoisomers and geometric isomers, both individually and in any and all possible mixtures. In each of the foregoing and following embodiments, it is also to be understood that the formulae include and represent any and all crystalline forms, partially crystalline forms, and non-crystalline and/or amorphous forms of the compounds.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

Illustrative formats for oral administration include tablets, capsules, elixirs, syrups, and the like. Illustrative routes for parenteral administration include intravenous, intraarterial, intraperitoneal, epidural, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. In cases where the compound in itself is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill

Depending upon the route of administration, a wide range of permissible dosages are contemplated herein, including doses falling in the range from about 1 μg/kg to about 1 g/kg. The dosages may be single or divided, and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In addition to the illustrative dosages and dosing protocols described herein, it is to be understood that an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The term “patient” includes human and non-human animals such as companion animals (dogs and cats and the like) and livestock animals. Livestock animals are animals raised for food production. The patient to be treated is preferably a mammal, in particular a human being.

It has been suggested that resistance to antibiotics has developed over the years via a myriad of processes including the inordinate use of antibiotics and the lack of development of new antibiotics³. The wide gap between emergence of drug-resistant pathogens and the development of novel antibacterial therapeutics has been attributed to the non-profitable nature of the venture (it costs several millions of dollars to conduct clinical trials and the high probability of bacterial resistance emerging against a new antibiotic hinders investment in antibiotic discovery)^{2, 3}. Efforts however, need to be directed towards identifying and developing novel structures as antibacterial agents with possibly novel mechanisms of action². It is projected that in the absence of new antibacterial agents, annual mortality rates could exceed 10 million by the year 2050.

The following non-limiting exemplary embodiments are included herein to further illustrate the invention. These exemplary embodiments are not intended and should not be interpreted to limit the scope of the invention in any way. It is also to be understood that numerous variations of these exemplary embodiments are contemplated herein.

Materials and Methods

General Chemistry Considerations. Unless noted otherwise, all reagents and solvents were purchased from commercial sources and used as received. The ¹H and ¹³C NMR spectra were obtained in CDCl₃ as solvent using a 500 MHz spectrometer with Me₄Si as an internal standard. Chemical shifts are reported in parts per million (δ) and are calibrated using residual undeuterated solvent as an internal reference. Data for ¹H NMR spectra are reported as follows: chemical shift (δ ppm) (multiplicity, coupling constant (Hz), integration). Multiplicities are reported as follows: s=singlet, d=doublet, t=triplet, q=quartet, m=multiplet, or combinations thereof. High resolution mass spectra (HRMS) were obtained using electron spray ionization (ESI) technique and as TOF mass analyzer. New compounds were characterized by ¹H NMR, ¹³C NMR, and HRMS data.

Biological Evaluation, Bacterial Viability Analysis:

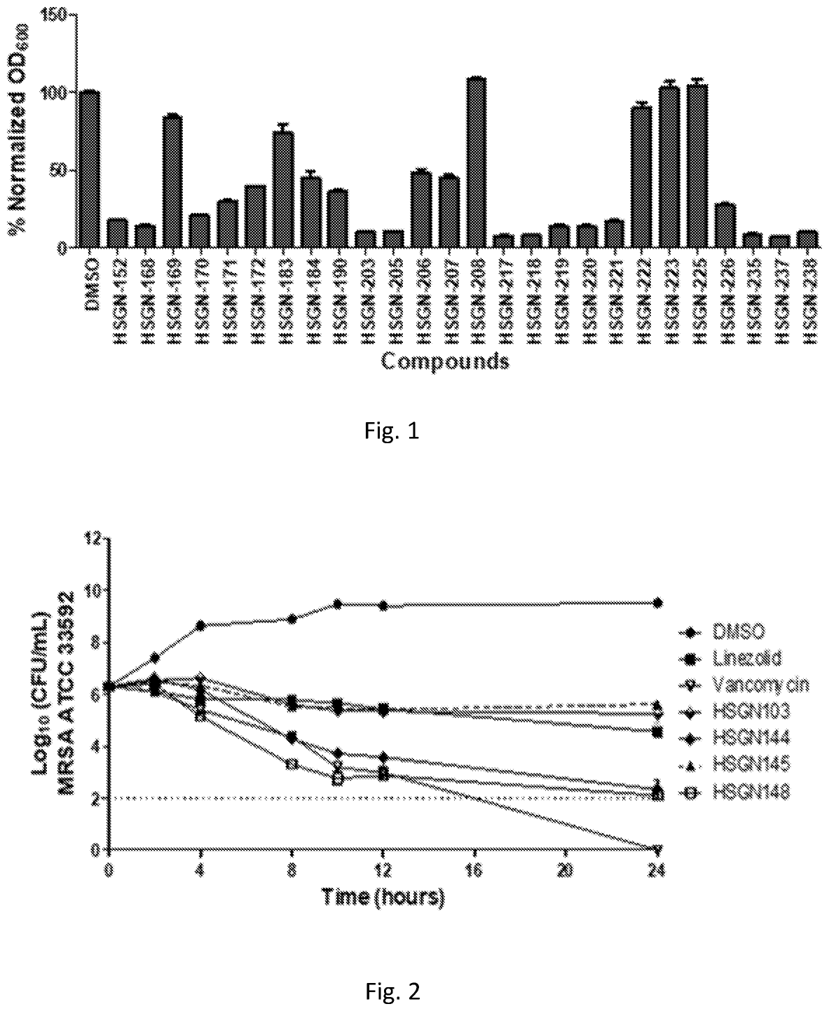

S. aureus culture was grown overnight in 10 ml Tryptic Soy Broth (TSB). The overnight culture was diluted 1:1,000 in Cation-adjusted Mueller-Hinton broth (MHB) and grown to early exponential phase by incubation at 37° C. Aliquots of bacteria culture were then dispensed into sterile glass tubes containing stock solutions of compounds in DMSO to yield a final concentration of 16 μg/mL (FIG. 1).

All MRSA isolates were acquired from BEI Resources. The remaining bacteria were purchased from the American Type Culture Collection (ATCC).

Determination of the MIC and MBC

The minimum inhibitory concentration (MIC) of compounds and control antibiotics (methicillin, linezolid and vancomycin), tested from 128 μg/mL to 1 μg/mL, was determined using the broth microdilution method²⁸ (Reference: Clinical and Laboratory Standards Institute (2012) Methods for Dilution Antimicrobial Susceptibility Tests for Bacteria That Grow Aerobically—Ninth Edition: Approved Standard M07-A9. Wayne, Pa.) against the selected bacterial pathogens. Bacteria were cultured in cation-adjusted Mueller Hinton Broth (for strains in Tables 1-3) or Brain Heart Infusion broth (for Enterococcus faecium) or Tryptic Soy Broth (all other bacteria) in a 96-well plate at 37° C. for at least 20 hours. The MIC was classified as the lowest concentration where no visual growth of bacteria was observed. The minimum bactericidal concentration (MBC) was tested by spotting 4 μL from wells with no growth onto Tryptic Soy Agar (TSA) plates. Plates were incubated at 37° C. for at least 18 hours before recording the MBC.

Time-kill analysis (H. Mohammad et al., PLoS One. 2017, 12(8):e0182821; M. Hagras et al., Eur J Med Chem. 2018, 143:1448-1456.)

The time-kill analysis was performed as previously described²⁹. MRSA USA300 cells in logarithmic growth phase were diluted to 1.25×10⁶ colony-forming units per mL (CFU/mL) and exposed to concentrations equivalent to either 3× MIC or 6× MIC (in triplicate) of compound F₆ or linezolid in Tryptic Soy Broth. Aliquots (100 μL) were collected from each treatment after 0, 2, 4, 8, 12, and 24 hours of incubation at 37° C. and subsequently serially diluted in phosphate-buffered saline (PBS). Bacteria were then transferred to TSA plates and incubated at 37° C. for 18-20 hours before viable CFU/mL was determined (see FIG. 2).

Results and Discussions

TABLE 1
MIC values (in μg/mL) for Compounds against
a panel of drug-resistant Gram-positive bacteria

| | | | E. | | |
|---|---|---|---|---|---|
| | | MRSA | faecalis | VRE | L. |
| | S. | ATCC | ATCC | ATCC | monocytogenes |
| Compounds | aureus | 33592 | 29212 | 51575 | ATCC 19115 |
| HSGN143 | 32 | 32 | — | — | — |
| HSGN144 | 0.25 | 0.5 | 2 | 2 | 1 |
| HSGN145 | 1 | 1 | >8 | 8 | 8 |
| HSGN-148 | 1 | 0.5 | 4 | 4 | 2 |
| HSGN-152 | 2 | 2 | — | — | — |
| HSGN-168 | 16 | 8 | — | — | — |
| HSGN-169 | >64 | >64 | — | — | — |
| HSGN-170 | 4 | 2 | — | — | — |
| HSGN-171 | 16 | 8 | — | — | — |
| HSGN-172 | 64 | 32 | — | — | — |
| HSGN-183 | >64 | >64 | — | — | — |
| HSGN-184 | 16 | 16 | — | — | — |
| HSGN-190 | 32 | 32 | — | — | — |
| HSGN-203 | 0.25 | 0.25 | 1 | 0.5 | 0.5 |
| HSGN-204 | 0.125 | 0.125 | 0.25 | 0.25 | 0.125 |
| HSGN-205 | 0.5 | 0.25 | 2 | 4 | 1 |
| HSGN-206 | 32 | 32 | — | — | — |
| HSGN-207 | 16 | 8 | — | — | — |
| HSGN-210 | 0.25 | 0.25 | 0.5 | 0.5 | 0.25 |
| HSGN-217 | 0.5 | 0.5 | — | — | — |
| HSGN-218 | 0.0625 | 0.0625 | 0.25 | 0.25 | 0.125 |
| HSGN-219 | 0.5 | 0.5 | — | — | — |
| HSGN-220 | 0.125 | 0.25 | — | — | — |
| HSGN-221 | 4 | 4 | — | — | — |
| HSGN-ccc | 32 | 32 | — | — | — |
| HSGN-226 | 16 | 16 | — | — | — |
| HSGN-235 | 1 | 2 | 4 | 2 | 1 |
| HSGN-237 | 0.5 | 0.5 | 1 | 1 | 0.5 |
| HSGN-238 | 0.25 | 0.25 | 1 | 1 | 0.5 |
| HSGN-247 | 0.5 | 0.5 | — | — | — |
| HSGN-248 | 0.5 | 0.25 | — | — | — |
| HSGN-250 | 4 | 2 | — | — | — |
| HSGN-251 | 16 | 8 | — | — | — |
| HSGN-252 | 2 | 1 | — | — | — |
| HSGN-253 | 0.25 | 0.125 | — | — | — |
| HSGN-255 | >64 | >64 | — | — | — |
| HSGN-256 | >64 | >64 | — | — | — |
| HSGN-257 | >64 | >64 | — | — | — |
| HSGN-258 | 2 | 2 | — | — | — |
| HSGN-259 | 0.0625 | 0.0313 | — | — | — |
| HSGN-260 | >64 | >64 | — | — | — |
| HSGN-262 | 16 | 16 | — | — | — |
| HSGN-265 | 0.0625 | 0.0625 | — | — | — |
| HSGN-266 | 0.5 | 0.5 | — | — | — |
| HSGN-280 | 1 | 1 | — | — | — |
| HSGN-281 | 0.0625 | 0.0625 | — | — | — |
| HSGN-282 | 0.0625 | 0.0625 | — | — | — |
| HSGN-283 | 0.0625 | 0.0625 | — | — | — |
| HSGN-2111 | 1 | 1 | — | — | — |
| HSGN-2112 | 0.5 | 0.5 | — | — | — |
| HSGN-2113 | 0.25 | 0.25 | — | — | — |
| HSGN-2114 | 0.5 | 0.5 | — | — | — |
| HSGN-2115 | 0.5 | 0.5 | — | — | — |
| HSGN-2135 | >128 | >128 | — | — | — |
| HSGN-2136 | >128 | >128 | — | — | — |
| HSGN-2140 | 1 | 1 | — | — | — |
| HSGN-2141 | 32 | 32 | — | — | — |
| HSGN-2142 | 1 | 0.5 | — | — | — |
| HSGN-2143 | 2 | 1 | — | — | — |

TABLE 2
MIC (μg/mL) of compounds against a panel of C. difficile strains.

| | | | | | | | | C. |
|---|---|---|---|---|---|---|---|---|
| | C. | C. | C. | C. | C. | C. | C. | difficile |
| | difficile | difficile | difficile | difficile | difficile | difficile | difficile | ATCC |
| Compounds/ | NR-13432 | NR-13435 | NR-32883 | NR-32891 | NR-32895 | NR-32904 | ATCC | BAA |
| Control drug | (isolate 6) | (isolate 9) | (P2) | (P13) | (P19) | (P30) | 43255 | 1801 |
| HSGN-103 | 0.5 | 0.5 | 0.5 | 0.5 | 0.5 | 0.5 | 0.5 | 0.5 |
| HSGN 144 | 0.015 | 0.015 | 0.003 | 0.125 | 0.06 | 0.125 | 0.125 | 0.03 |
| HSGN-145 | 0.5 | 0.5 | 1 | 2 | 1 | 1 | 0.5 | 0.5 |
| HSGN 148 | 0.5 | 0.5 | 0.5 | 0.5 | 0.5 | 0.5 | 0.5 | 0.5 |
| HSGN 203 | 0.125 | 0.06 | 0.06 | 0.125 | 0.06 | 0.125 | 0.25 | 0.125 |
| HSGN 204 | 0.03 | 0.007 | 0.003 | 0.03 | 0.007 | 0.015 | 0.003 | 0.03 |
| HSGN 205 | 0.03 | 0.007 | 0.003 | 0.03 | 0.007 | 0.015 | 0.003 | 0.03 |
| HSGN 210 | 0.015 | 0.003 | 0.003 | 0.06 | 0.03 | 0.06 | 0.007 | 0.06 |
| HSGN 217 | 0.06 | 0.06 | 0.06 | 0.125 | 0.125 | 0.125 | 0.06 | 0.06 |
| HSGN 218 | 0.03 | 0.003 | 0.007 | 0.007 | 0.007 | 0.007 | 0.015 | 0.03 |
| HSGN 219 | 0.03 | 0.007 | 0.003 | 0.125 | 0.06 | 0.06 | 0.06 | 0.015 |
| HSGN 235 | 0.06 | 0.06 | 0.06 | 0.25 | 0.06 | 0.25 | 0.06 | 0.125 |
| HSGN 237 | 0.06 | 0.03 | 0.03 | 0.125 | 0.03 | 0.03 | 0.03 | 0.06 |
| HSGN 238 | 0.03 | 0.015 | 0.007 | 0.06 | 0.015 | 0.015 | 0.06 | 0.03 |
| HSGN-247 | — | — | — | — | — | — | — | 0.015 |
| HSGN-248 | — | — | — | — | — | — | — | 0.015 |
| HSGN-259 | — | — | — | — | — | — | — | <0.003 |
| Vancomycin | 0.25 | 1 | 0.5 | 0.5 | 1 | 1 | 1 | 1 |

TABLE 3
Results from Time-Kill Assay. HSGN-103
and HSGN-145 are bacteriostatic; while
HSGN-144 and HSGN-148 are bactericidal.

| Test Agent | Δlog₂ CFU/mL | Bactericidal/Bacteriostatic |
|---|---|---|
| Linezolid | 1.7 | Bacteriostatic |
| HSGN-103 | 1.04 | Bacteriostatic |
| HSGN-145 | 0.69 | Bacteriostatic |
| HSGN-144 | 3.96 | Bactericidal |
| HSGN-148 | 4.2 | Bactericidal |
| Vancomycin | — | Bactericidal |

TABLE 4
MIC (μg/mL) of compounds against
Neisseria gonorrhea strain 181.

| Compounds/Control | Neisseria gonorrhea |
|---|---|
| antibiotics | strain 181 |
| HSGN 148 | 0.06 |
| HSGN 203 | 0.5 |
| HSGN 204 | 0.25 |
| HSGN 205 | 0.25 |
| HSGN 210 | 0.5 |
| HSGN 217 | 0.25 |
| HSGN 218 | 0.125 |
| HSGN 219 | 2 |
| HSGN 235 | 16 |
| HSGN 237 | 0.125 |
| HSGN 238 | 0.125 |
| HSGN 247 | 0.125 |
| HSGN 248 | >128 |
| HSGN 253 | 2 |
| HSGN 259 | 1 |
| HSGN 265 | 2 |
| Azithromycin | 256 |
| Tetracycline | 2 |

General synthetic scheme is shown in Scheme 1 below.

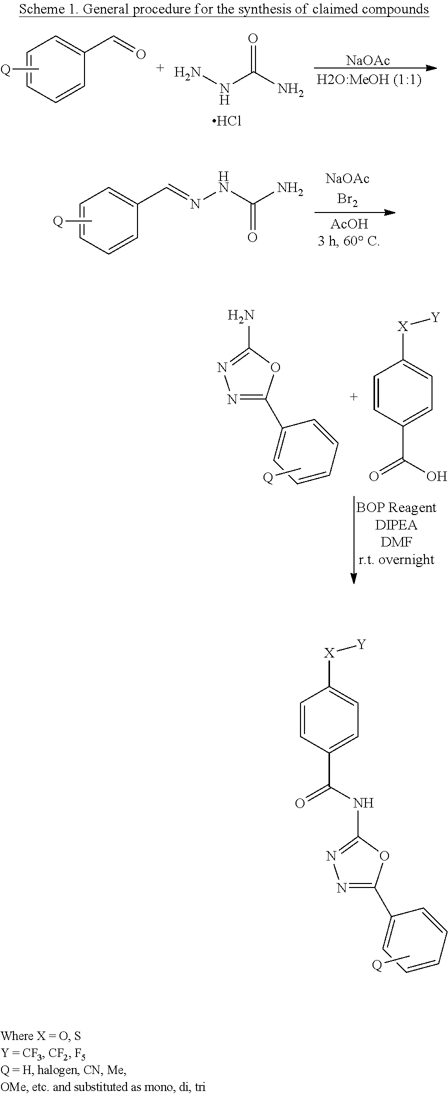

### 1,3,4-oxadiazolyl amine synthesis:

Step 1: To a vial was added the semicarbazide hydrochloride (1.0 eq.) and sodium acetate (2.0 eq.) followed by water (10 mL). To a second vial was added the benzaldehyde (1.0 eq.) in methanol (10 mL). The aldehyde solution was then added dropwise to the first solution while stirring at room temperature. The reaction stirred for 30 min in which time it formed a suspension. The solid was filtered out by vacuum filtration.

Step 2: To a vial was added the imine formed in Step 1 (1.0 eq.), sodium acetate (2.0 eq.) and acetic acid (0.5 mL). To a second vial was added the bromine (1.1 eq.) in acetic acid (0.5 mL). The bromine solution was then added to the first vial while stirring in a dropwise manner at room temperature. The reaction was then heated to 60° C. and stirred for 3 h. The reaction was then cooled to room temperature and poured onto ice water. A yellow precipitate formed and was filtered by vacuum filtration then rinsed with DCM to provide the corresponding 1,3,4-oxadiazolyl amine.

Amide Coupling of Compounds:

A 20 mL screw caped vial, charged with the corresponding acid (0.5 mmol), amine (0.5 mmol), BOP reagent (1.4 mmol) and diisopropylethylamine (13 mmol) in anhydrous DMF solvent (3 mL) was stirred at room temperature for 16 h. After completion, the reaction mixture was concentrated under reduced pressure, followed by flash column chromatography (hexanes:ethyl acetate 80:20 to 60:40) give the desired product.

### 4-(difluoromethoxy)-N-(5-(thiophen-2-yl)-1,3,4-oxadiazol-2-yl)benzamide (HSGN-143)

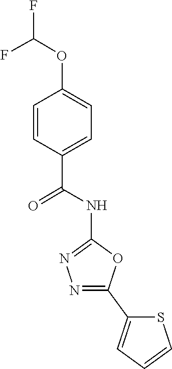 [SMILES: O=C(Nc1nnc(-c2cccs2)o1)c1ccc(OC(F)F)cc1]

¹H NMR (500 MHz, DMSO-d₆) δ 12.17 (s, 1H), 8.11-8.06 (m, 2H), 7.92 (dd, J=5.0, 1.2 Hz, 1H), 7.75 (dd, J=3.8, 1.2 Hz, 1H), 7.33 (d, J=8.7 Hz, 2H), 7.29-7.26 (m, 1H).¹³C NMR (201 MHz, DMSO-d₆) δ 164.9, 157.9, 154.8, 131.6, 131.2, 130.2, 129.3, 129.2, 124.7, 118.4, 116.4 (t, J=258.3 Hz). HRMS (ESI) m/z calcd for C₁₄H₁₀F₂N₃O₃S [M+H]⁺338.0408, found 338.0406.

### 4-(pentafluoro-sulfanyl)-N-(5-(thiophen-2-yl)-1,3,4-oxadiazol-2-yl)benzamide (HSGN-144):

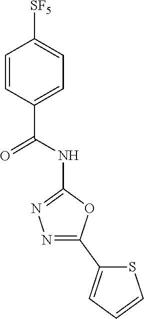

¹H NMR (500 MHz, DMSO-d₆) δ 8.21 (d, J=8.5 Hz, 2H), 8.10 (d, J=8.9 Hz, 2H), 7.93 (dd, J=5.0, 1.2 Hz, 1H), 7.76 (dd, J=3.7, 1.2 Hz, 1H), 7.28 (dd, J=5.0, 3.7 Hz, 1H). ¹³C NMR (126 MHz, DMSO-d₆) δ 165.0, 158.0, 157.3, 155.7, 136.8, 131.7, 130.3, 130.0, 129.2, 126.7, 124.6. HRMS (ESI) m/z calcd for C₁₃H₉F₅N₃O₂S₂ [M+H]⁺398.0053, found 398.0051.

### N-(5-(thiophen-2-yl)-1,3,4-oxadiazol-2-yl)-4-(trifluoromethoxy)benzamide (HSGN-145):

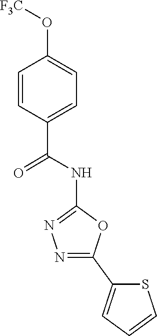 [SMILES: O=C(Nc1nnc(-c2cccs2)o1)c1ccc(OC(F)(F)F)cc1]

¹H NMR (500 MHz, DMSO-d₆) δ 12.28 (s, 1H), 8.14 (d, J=8.4 Hz, 2H), 7.93 (dd, J=5.0, 1.2 Hz, 1H), 7.76 (d, J=3.7 Hz, 1H), 7.55 (d, J=8.3 Hz, 2H), 7.28 (dd, J=5.0, 3.7 Hz, 1H). ¹³C NMR (126 MHz, DMSO-d₆) δ 164.9, 157.9, 157.6, 151.8, 132.0, 131.6, 131.3, 130.2, 129.2, 124.7, 121.4 (q, J=258.3 Hz), 121.2. HRMS (ESI) m/z calcd for C₁₄H₁₀F₃N₃O₃S [M+H]⁺356.0313, found 356.0311.

### N-(5-(thiophen-2-yl)-1,3,4-oxadiazol-2-yl)-4-((trifluoromethyl)thio)benzamide (HSGN-148):

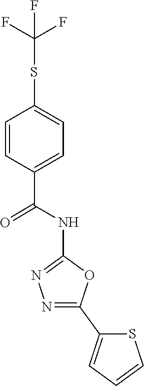 [SMILES: O=C(Nc1nnc(-c2cccs2)o1)c1ccc(SC(F)(F)F)cc1]

¹H NMR (500 MHz, DMSO-d₆) δ 8.12 (d, J=8.1 Hz, 2H), 7.92 (d, J=5.0 Hz, 1H), 7.89 (d, J=8.0 Hz, 2H), 7.75 (d, J=3.7 Hz, 1H), 7.28 (dd, J=5.0, 3.7 Hz, 1H). ¹³C NMR (126 MHz, DMSO-d₆) δ 165.3, 157.9, 157.5, 136.2, 135.6, 131.7, 131.1 (q, J=308.7 Hz), 130.2, 130.1, 129.2, 128.5, 124.7. HRMS (ESI) m/z calcd for C₁₄H₉F₃N₃O₂S₂ [M+H]⁺372.0084, found 372.0082.

### N-(5-(thiophen-2-yl)-1,3,4-oxadiazol-2-yl)-5-(trifluoromethyl)thiazole-2-carboxamide (HSGN-168):

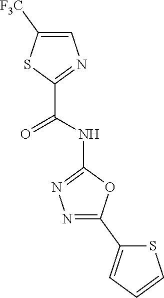 [SMILES: O=C(Nc1nnc(-c2cccs2)o1)c1ncc(C(F)(F)F)s1]

¹H NMR (500 MHz, DMSO-d₆) δ 8.58 (s, 1H), 7.87 (d, J=5.0 Hz, 1H), 7.72 (d, J=3.7 Hz, 1H), 7.25 (s, 1H). HRMS (ESI) m/z calcd for C₁₁H₆F₃N₄O₂S₂ [M+H]⁺346.9879, found 346.9877.

### N-(5-(thiophen-2-yl)-1,3,4-oxadiazol-2-yl)-5-(trifluoromethyl)furan-2-carboxamide (HSGN-169):

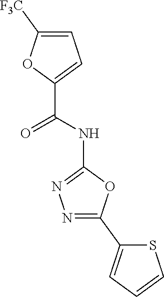 [SMILES: O=C(Nc1nnc(-c2cccs2)o1)c1ccc(C(F)(F)F)o1]

¹H NMR (500 MHz, DMSO-d₆) δ 7.93 (d, J=5.0 Hz, 1H), 7.75 (d, J=3.7 Hz, 1H), 7.68-7.63 (m, 1H), 7.47 (d, J=3.7 Hz, 1H), 7.32-7.21 (m, 1H). HRMS (ESI) m/z calcd for C₁₂H₇F₃N₃O₃S [M+H]⁺330.0154, found 330.0154.

### N-5-(thiophen-2-yl)-1,3,4-oxadiazol-2-yl)-5-(trifluoromethyl)thiophene-2-carboxamide (HSGN-170):

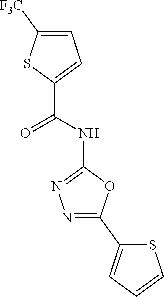 [SMILES: O=C(Nc1nnc(-c2cccs2)o1)c1ccc(C(F)(F)F)s1]

¹H NMR (500 MHz, DMSO-d₆) δ 8.09 (s, 1H), 7.93 (dd, J=5.0, 1.2 Hz, 1H), 7.84-7.81 (m, 1H), 7.80-7.72 (m, 1H), 7.28 (dd, J=5.0, 3.7 Hz, 1H). ¹³C NMR (201 MHz, DMSO-d₆) δ 161.0, 157.9, 143.2, 134.9 (q, J=39 Hz), 131.8, 131.5, 131.3, 131.2, 130.4, 129.1, 124.4, 123.0 (q, J=269.6 Hz). HRMS (ESI) m/z calcd for C₁₂H₇F₃N₃O₂S₂ [M+H]⁺345.9926, found 345.9924.

### N-(5-(thiophen-2-yl)-1,3,4-oxadiazol-2-yl)-6-(trifluoromethyl)nicotinamide (HSGN-171):

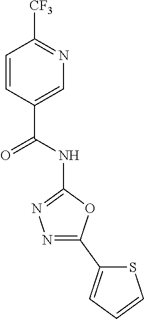 [SMILES: O=C(Nc1nnc(-c2cccs2)o1)c1ccc(C(F)(F)F)nc1]

¹H NMR (500 MHz, DMSO-d₆) δ 9.30 (d, J=2.1 Hz, 1H), 8.62 (dd, J=8.1, 2.1 Hz, 1H), 8.11 (d, J=8.2 Hz, 1H), 7.94 (dd, J=5.0, 1.2 Hz, 1H), 7.82-7.76 (m, 1H), 7.29 (dd, J=5.0, 3.7 Hz, 1H). HRMS (ESI) m/z calcd for C₁₃H₈F₃N₄O₂S [M+H]⁺341.0315, found 341.0313.

### N-(5-(thiophen-2-yl)-1,3,4-oxadiazol-2-yl)-5-(trifluoromethyl)picolinamide (HSGN-172):

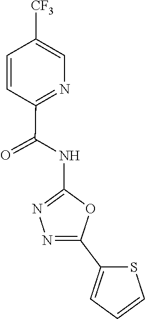 [SMILES: O=C(Nc1nnc(-c2cccs2)o1)c1ccc(C(F)(F)F)cn1]

¹H NMR (500 MHz, DMSO-d₆) δ 12.35 (s, 1H), 9.14 (d, J=2.4 Hz, 1H), 8.52 (dd, J=8.3, 2.3 Hz, 1H), 8.33 (d, J=8.2 Hz, 1H), 7.94 (dd, J=5.0, 1.3 Hz, 1H), 7.77 (dd, J=3.7, 1.3 Hz, 1H), 7.29 (dd, J=5.0, 3.7 Hz, 1H).¹³C NMR (126 MHz, DMSO-d₆) δ 162.6, 158.6, 157.0, 152.0, 146.2, 136.5, 131.9, 130.4, 129.2, 128.8 (q, J=32.7 Hz), 124.8 (q, J=274.6 Hz), 124.6, 124.1. HRMS (ESI) m/z calcd for C₁₃H₈F₃N₄O₂S [M+H]⁺341.0315, found 341.0315.

### N-(5-(thiophen-2-yl)-1,3,4-oxadiazol-2-yl)-3-(trifluoromethyl)-1H-pyrazole-5-carboxamide (HSGN-183):

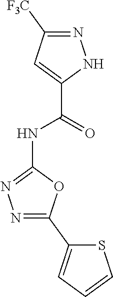 [SMILES: O=C(Nc1nnc(-c2cccs2)o1)c1cc(C(F)(F)F)n[nH]1]

¹H NMR (500 MHz, DMSO-d₆) δ 7.94-7.92 (m, 1H), 7.75 (dd, J=3.7, 1.2 Hz, 1H), 7.61-7.55 (m, 1H), 7.28 (dd, J=5.0, 3.7 Hz, 1H).¹³C NMR (126 MHz, DMSO-d₆) δ 163.8, 157.1, 142.1 (q, J=36.5 Hz), 131.8, 130.3, 129.2, 127.7, 122.7 (q, J=269.6 Hz), 118.4, 106.1. HRMS (ESI) m/z calcd for C₁₁H₇F₃N₅O₂S [M+H]⁺330.0267, found 330.0266.

### N-(5-(thiophen-2-yl)-1,3,4-oxadiazol-2-yl)-6-(trifluoromethyl)picolinamide (HSGN-184):

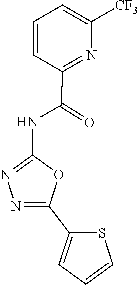 [SMILES: O=C(Nc1nnc(-c2cccs2)o1)c1cccc(C(F)(F)F)n1]

1H NMR (500 MHz, DMSO-d₆) δ 9.41 (s, 1H), 9.20 (s, 1H), 8.75 (s, 1H), 7.96-7.91 (m, 1H), 7.76 (d, J=2.5 Hz, 1H), 7.29-7.26 (m, 1H).¹³C NMR (126 MHz, DMSO-d₆) δ 164.1, 157.8, 157.0, 153.6, 149.8, 133.8, 131.8, 130.3, 129.2, 129.1, 125.5 (q, J=32.7 Hz), 124.8 (q, J=274.6 Hz), 124.5. HRMS (ESI) m/z calcd for C₁₃H₈F₃N₄O₂S [M+H]⁺341.0315, found 341.0314.

### N-(5-(thiophen-2-yl)-1,3,4-oxadiazol-2-yl)-2-(trifluoromethyl)pyrimidine-5-carboxamide (HSGN-190):

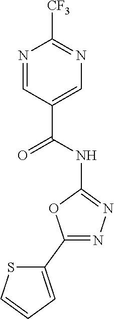 [SMILES: O=C(Nc1nnc(-c2cccs2)o1)c1cnc(C(F)(F)F)nc1]

¹H NMR (500 MHz, DMSO-d₆) δ 9.53 (s, 2H), 7.95 (dd, J=5.0, 1.2 Hz, 1H), 7.78 (dd, J=3.7, 1.2 Hz, 1H), 7.29 (dd, J=5.0, 3.7 Hz, 1H). HRMS (ESI) m/z calcd for C₁₂H₇F₃N₅O₂S [M+H]⁺342.0273, found 342.0274.

### N-(5-(4-(trifluoromethyl)phenyl)-1,3,4-oxadiazol-2-yl)-4-((trifluoromethyl)thio)benzamide (HSGN-203):

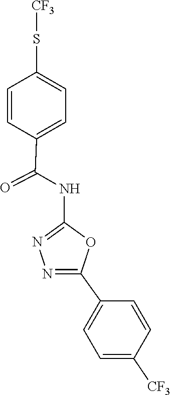 [SMILES: O=C(Nc1nnc(-c2ccc(C(F)(F)F)cc2)o1)c1ccc(SC(F)(F)F)cc1]

¹H NMR (500 MHz, DMSO-d₆) δ 8.15 (dd, J=14.0, 7.9 Hz, 4H), 7.98 (d, J=8.0 Hz, 2H), 7.90 (d, J=8.0 Hz, 2H). HRMS (ESI) m/z calcd for C₁₇H₁₀F₆N₃O₂S [M+H]⁺434.0398, found 434.0396.

### N-(5-(3-chlorophenyl)-1,3,4-oxadiazol-2-yl)-4-((trifluoromethyl)thio)benzamide (HSGN-204):

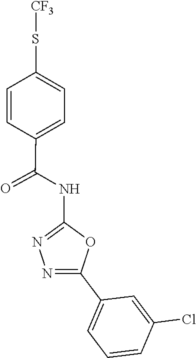 [SMILES: O=C(Nc1nnc(-c2cccc(Cl)c2)o1)c1ccc(SC(F)(F)F)cc1]

¹H NMR (500 MHz, DMSO-d₆) δ 8.13 (d, J=8.0 Hz, 2H), 7.93-7.87 (m, 4H), 7.75-7.68 (m, 1H), 7.64 (t, J=7.8 Hz, 1H). ¹³C NMR (126 MHz, DMSO) δ 163.68, 161.74, 136.27, 132.46, 131.15, 130.16, 128.70, 125.78, 122.85, 119.22, 113.36, 113.17. HRMS (ESI) m/z calcd for C₁₆H₁₀ClF₃N₃O₂S [M+H]⁺400.0134, found 400.0132.

### N-(5-(3-fluorophenyl)-1,3,4-oxadiazol-2-yl)-4-((trifluoromethyl)thio)benzamide (HSGN-205):

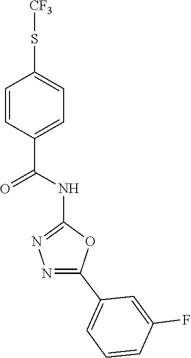 [SMILES: O=C(Nc1nnc(-c2cccc(F)c2)o1)c1ccc(SC(F)(F)F)cc1]

¹H NMR (500 MHz, DMSO-d₆) δ 8.13 (d, J=8.0 Hz, 2H), 7.89 (d, J=8.1 Hz, 2H), 7.80 (d, J=7.8 Hz, 1H), 7.73-7.63 (m, 2H), 7.49 (td, J=8.6, 2.7 Hz, 1H). ¹³C NMR (126 MHz, DMSO) δ 163.68, 161.74, 136.27, 132.46, 131.15, 130.16, 128.70, 125.78, 122.85, 119.22, 113.36, 113.17. HRMS (ESI) m/z calcd for C₁₆H₁₀F₄N₃O₂S [M+H]⁺384.0430, found 384.0429.

### N-(5-(pyridin-3-yl)-1,3,4-oxadiazol-2-yl)-4-((trifluoromethyl)thio)benzamide (HSGN-206):

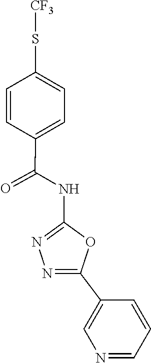 [SMILES: O=C(Nc1nnc(-c2cccnc2)o1)c1ccc(SC(F)(F)F)cc1]

¹H NMR (500 MHz, DMSO-d₆) δ 9.12 (d, J=2.2 Hz, 1H), 8.79 (dd, J=4.8, 1.6 Hz, 1H), 8.31 (dt, J=8.0, 1.9 Hz, 1H), 8.13 (d, J=8.0 Hz, 2H), 7.90 (d, J=8.0 Hz, 2H), 7.64 (dd, J=8.0, 4.8 Hz, 1H). HRMS (ESI) m/z calcd for C₁₅H₁₀F₃N₄O₂S [M+H]⁺367.0477, found 367.0479.

### N-(5-(4-methoxyphenyl)-1,3,4-oxadiazol-2-yl)-4-((trifluoromethyl)thio)benzamide (HSGN-207):

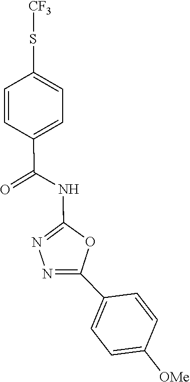 [SMILES: COc1ccc(-c2nnc(NC(=O)c3ccc(SC(F)(F)F)cc3)o2)cc1]

¹H NMR (500 MHz, DMSO-d₆) δ 12.30 (s, 1H), 8.13 (d, J=7.8 Hz, 2H), 7.89 (d, J=8.9 Hz, 3H), 7.14 (d, J=8.9 Hz, 2H), 3.84 (s, 3H). HRMS (ESI) m/z calcd for C₁₇H₁₃F₃N₃O₃S [M+H]⁺396.0630, found 396.0629.

### N-(5-(thiophen-2-yl)-4H-1,2,4-triazol-3-yl)-4-((trifluoromethyl)thio)benzamide (HSGN-208):

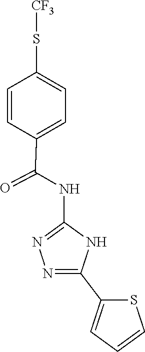 [SMILES: O=C(Nc1nnc(-c2cccs2)[nH]1)c1ccc(SC(F)(F)F)cc1]

¹H NMR (500 MHz, DMSO-d₆) δ 8.17 (d, J=8.4 Hz, 2H), 7.89 (d, J=8.3 Hz, 2H), 7.67 (dd, J=5.0, 1.2 Hz, 1H), 7.58 (dd, J=3.6, 1.2 Hz, 1H), 7.14 (dd, J=5.0, 3.6 Hz, 1H).¹³C NMR (126 MHz, DMSO) δ 166.90, 159.16, 156.75, 135.56, 135.14, 133.25, 132.23, 129.19, 128.54, 128.18.

### N-(5-(5-chlorothiophen-2-yl)-1,3,4-oxadiazol-2-yl)-4-((trifluoromethyl)thio)benzamide (HSGN-210)

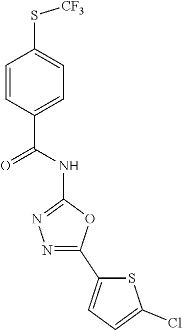 [SMILES: O=C(Nc1nnc(-c2ccc(Cl)s2)o1)c1ccc(SC(F)(F)F)cc1]

¹H NMR (500 MHz, DMSO-d₆) δ 8.11 (d, J=8.0 Hz, 2H), 7.89 (d, J=8.0 Hz, 2H), 7.63 (d, J=4.0 Hz, 1H), 7.34 (d, J=4.1 Hz, 1H). HRMS (ESI) m/z calcd for C₁₄H₈ClF₃N₃O₂S₂ [M+H]⁺405.9699, found 405.9700.

### N-(5-(3-bromophenyl)-1,3,4-oxadiazol-2-yl)-4-((trifluoromethyl)thio)benzamide (HSGN-217)

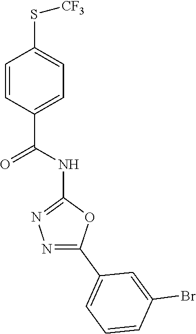 [SMILES: O=C(Nc1nnc(-c2cccc(Br)c2)o1)c1ccc(SC(F)(F)F)cc1]

¹H NMR (500 MHz, DMSO-d₆) δ 8.13 (d, J=7.9 Hz, 2H), 8.06 (s, 1H), 7.97-7.81 (m, 4H), 7.57 (t, J=7.9 Hz, 1H). HRMS (ESI) m/z calcd for C₁₆H₁₀BrF₃N₃O₂S [M+H]⁺443.9629, found 443.9630.

### N-(5-(3,5-dichlorophenyl)-1,3,4-oxadiazol-2-yl)-4-((trifluoromethyl)thio)benzamide (HSGN-218):

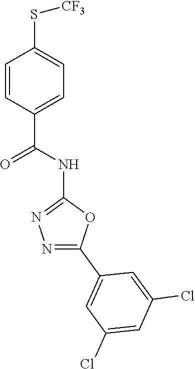 [SMILES: O=C(Nc1nnc(-c2cc(Cl)cc(Cl)c2)o1)c1ccc(SC(F)(F)F)cc1]

¹H NMR (500 MHz, DMSO-d₆) δ 8.12 (d, J=7.9 Hz, 2H), 7.90 (dd, J=10.4, 1.9 Hz, 5H). HRMS (ESI) m/z calcd for C₁₆H₉Cl₂F₃N₃O₂S [M+H]⁺433.9745, found 433.9744.

### N-(5-(2,4-difluorophenyl)-1,3,4-oxadiazol-2-yl)-4-((trifluoromethyl)thio)benzamide (HSGN-219):

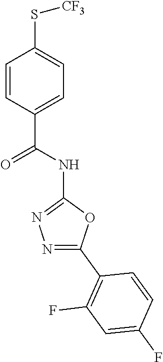 [SMILES: O=C(Nc1nnc(-c2ccc(F)cc2F)o1)c1ccc(SC(F)(F)F)cc1]

¹H NMR (500 MHz, DMSO-d₆) δ 8.12 (d, J=7.9 Hz, 2H), 8.04 (td, J=8.5, 6.3 Hz, 1H), 7.89 (d, J=8.1 Hz, 2H), 7.58 (ddd, J=11.4, 9.3, 2.5 Hz, 1H), 7.38-7.31 (m, 1H). HRMS (ESI) m/z calcd for C₁₆H₉F₅N₃O₂S [M+H]⁺402.0335, found 402.0333.

4-(trifluoromethoxy)-N-(5-(4-(trifluoromethyl)phenyl)-1,3,4-oxadiazol-2-yl)benzamide (HSGN-220):

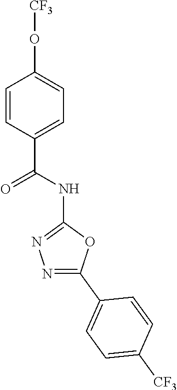 [SMILES: O=C(Nc1nnc(-c2ccc(C(F)(F)F)cc2)o1)c1ccc(OC(F)(F)F)cc1]

¹H NMR (500 MHz, DMSO-d₆) δ 8.16 (d, J=8.1 Hz, 4H), 7.98 (d, J=8.1 Hz, 2H), 7.55 (d, J=8.3 Hz, 2H). HRMS (ESI) m/z calcd for C₁₇H₁₀F₅N₃O₃ [M+H]⁺418.0626, found 418.0625.

### N-(3-(4-(trifluoromethyl)phenyl)-1,2,4-oxadiazol-5-yl)-4-((trifluoromethyl)thio)benzamide (HSGN-223):

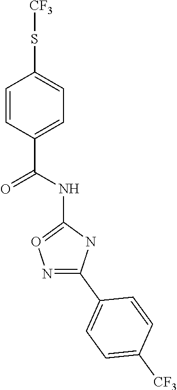

¹H NMR (500 MHz, DMSO-d₆) δ 7.97 (d, J=8.4 Hz, 2H), 7.70 (d, J=8.4 Hz, 2H), 7.53 (ddd, J=8.1, 6.9, 1.0 Hz, 2H), 7.40 (ddd, J=8.1, 6.9, 1.1 Hz, 2H).

### N-(5-(thiophen-2-yl)-1,3,4-thiadiazol-2-yl)-4-((trifluoromethyl)thio)benzamide (HSGN-225):

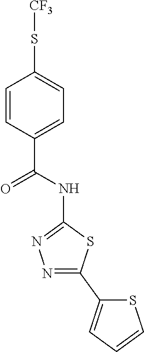 [SMILES: O=C(Nc1nnc(-c2cccs2)s1)c1ccc(SC(F)(F)F)cc1]

¹H NMR (500 MHz, DMSO-d₆) δ 8.21 (d, J=8.5 Hz, 2H), 7.86 (d, J=8.1 Hz, 2H), 7.58-7.53 (m, 2H), 7.50 (dd, J=5.0, 1.1 Hz, 1H), 7.10 (dd, J=5.1, 3.6 Hz, 1H). ¹³C NMR (126 MHz, DMSO) δ 164.80, 158.81, 144.53, 138.75, 136.15, 134.88, 131.15, 130.06, 128.70, 128.52, 128.30, 126.09, 124.43, 107.76.

3-fluoro-4-(trifluoromethoxy)-N-(5-(4-(trifluoromethoxy)phenyl)-1,3,4-oxadiazol-2-yl)benzamide (HSGN-235):

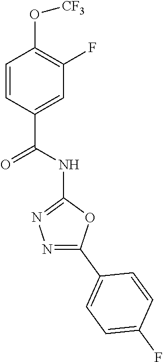 [SMILES: O=C(Nc1nnc(-c2ccc(F)cc2)o1)c1ccc(OC(F)(F)F)c(F)c1]

¹H NMR (500 MHz, DMSO-d₆) δ 8.14 (d, J=8.1 Hz, 2H), 7.98 (d, J=8.1 Hz, 2H), 7.81 (d, J=4.7 Hz, 2H), 7.47 (t, J=8.0 Hz, 1H).

### N-(5-(3-fluorophenyl)-1,3,4-oxadiazol-2-yl)-4-(trifluoromethoxy)benzamide (HSGN-237):

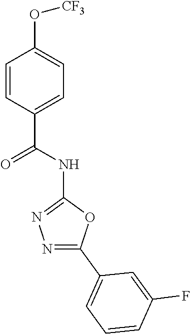 [SMILES: O=C(Nc1nnc(-c2cccc(F)c2)o1)c1ccc(OC(F)(F)F)cc1]

¹H NMR (500 MHz, DMSO-d₆) δ 8.18-8.14 (m, 2H), 7.80 (dd, J=7.8, 3.6 Hz, 1H), 7.74-7.64 (m, 2H), 7.59-7.46 (m, 3H). HRMS (ESI) m/z calcd for C₁₆H₁₀F₄N₃O₃ [M+H]⁺368.0658, found 368.0659.

### N-(5-(5-chlorothiophen-2-yl)-1,3,4-oxadiazol-2-yl)-4-(trifluoromethoxy)benzamide (HSGN-238):

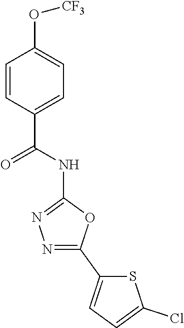 [SMILES: O=C(Nc1nnc(-c2ccc(Cl)s2)o1)c1ccc(OC(F)(F)F)cc1]

¹H NMR (500 MHz, DMSO-d₆) δ 8.14 (d, J=8.5 Hz, 2H), 7.62 (d, J=4.1 Hz, 1H), 7.54 (d, J=8.3 Hz, 2H), 7.33 (d, J=4.1 Hz, 1H). HRMS (ESI) m/z calcd for C₁₄H₈ClF₃N₃O₃S [M+H]⁺389.9927, found 389.9925.

### N-(5-(3-chlorophenyl)-1,3,4-oxadiazol-2-yl)-6-((trifluoromethyl)thio)nicotinamide (HSGN-247):

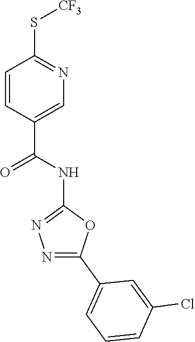 [SMILES: O=C(Nc1nnc(-c2cccc(Cl)c2)o1)c1ccc(SC(F)(F)F)nc1]

¹H NMR (500 MHz, DMSO-d₆) δ 9.17 (s, 1H), 8.44 (d, J=8.1 Hz, 1H), 7.92-7.79 (m, 3H), 7.69-7.60 (m, 2H).

### N-(5-(2,4-dichlorophenyl)-1,3,4-oxadiazol-2-yl)-4-((trifluoromethyl)thio)benzamide (HSGN-248):

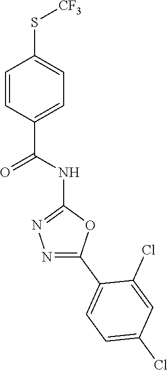 [SMILES: O=C(Nc1nnc(-c2ccc(Cl)cc2Cl)o1)c1ccc(SC(F)(F)F)cc1]

¹H NMR (500 MHz, DMSO-d₆) δ 8.12 (d, J=7.9 Hz, 2H), 7.96 (d, J=8.5 Hz, 1H), 7.94-7.87 (m, 3H), 7.68 (dd, J=8.4, 2.1 Hz, 1H). HRMS (ESI) m/z calcd for C₁₆H₉Cl₂F₃N₃O₂S [M+H]⁺433.9745, found 433.9746.

4-((difluoromethyl)thio)-N-(5-(thiophen-2-yl)-1,3,4-oxadiazol-2-yl)benzamide (HSGN-250):

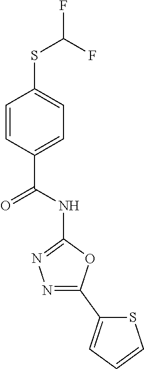 [SMILES: O=C(Nc1nnc(-c2cccs2)o1)c1ccc(SC(F)F)cc1]

¹H NMR (500 MHz, DMSO-d₆) δ 8.21 (d, J=8.5 Hz, 2H), 8.10 (d, J=8.9 Hz, 2H), 7.93 (dd, J=5.0, 1.2 Hz, 1H), 7.76 (dd, J=3.7, 1.2 Hz, 1H), 7.28 (dd, J=5.0, 3.7 Hz, 1H). ¹³C NMR (201 MHz, DMSO-d₆) δ 165.0, 157.6, 157.2, 133.2, 131.4, 131.0, 129.6, 129.2, 128.6, 124.2, 120.7 (t, J=273.4 Hz). HRMS (ESI) m/z calcd for C₁₃H₉F₅N₃O₂S₂ [M+H]⁺398.0053, found 398.0051.

4-(methylthio)-N-(5-(thiophen-2-yl)-1,3,4-oxadiazol-2-yl)benzamide (HSGN-251):

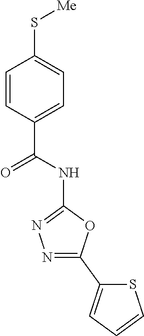 [SMILES: CSc1ccc(C(=O)Nc2nnc(-c3cccs3)o2)cc1]

¹H NMR (500 MHz, DMSO-d₆) δ 7.93 (dq, J=12.6, 7.6 Hz, 3H), 7.75 (t, J=6.8 Hz, 1H), 7.42-7.34 (m, 2H), 7.29-7.25 (m, 1H), 2.53 (d, J=11.9 Hz, 3H). ¹³C NMR (126 MHz, DMSO-d₆) δ 164.8, 158.0, 145.7, 131.6, 130.1, 129.3, 129.2, 128.4, 125.3, 124.8, 14.4. HRMS (ESI) m/z calcd for C₁₄H₁₂N₃O₂S₂ [M+H]⁺318.0371, found 318.0371.

### N-(5-(5-chloro-2-methoxyphenyl)-1,3,4-oxadiazol-2-yl)-4-((trifluoromethyl)thio)benzamide (HSGN-252):

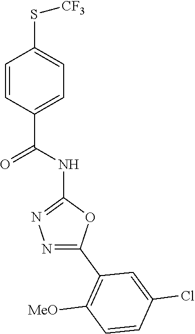 [SMILES: COc1ccc(Cl)cc1-c1nnc(NC(=O)c2ccc(SC(F)(F)F)cc2)o1]

¹H NMR (500 MHz, DMSO-d₆) δ 8.16-8.06 (m, 2H), 7.92-7.72 (m, 3H), 7.64 (t, J=10.9 Hz, 1H), 7.30 (q, J=10.3, 9.7 Hz, 1H), 3.89 (dd, J=16.3, 6.8 Hz, 3H).

### N-(5-(3-(trifluoromethyl)phenyl)-1,3 ,4-oxadiazol-2-yl)-4-((trifluoromethyl)thio)benzamide (HSGN-253):

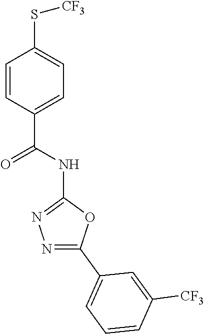 [SMILES: O=C(Nc1nnc(-c2cccc(C(F)(F)F)c2)o1)c1ccc(SC(F)(F)F)cc1]

¹H NMR (500 MHz, DMSO-d₆) δ 8.25 (d, J=7.9 Hz, 1H), 8.20-8.10 (m, 2H), 8.00 (d, J=7.8 Hz, 1H), 7.93-7.83 (m, 3H). HRMS (ESI) m/z calcd for C₁₇H₁₀F₆N₃O₂S [M+H]⁺434.0398, found 434.0399.

### N-(5-(3-chlorophenyl)-1,3,4-oxadiazol-2-yl)-4-(pentafluoro-δ6-sulfaneyl)benzenesulfonamide (HSGN-255):

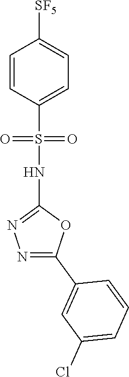

¹H NMR (500 MHz, DMSO-d₆) δ 7.86 (d, J=8.8 Hz, 4H), 7.78 (d, J=8.5 Hz, 4H).

### N-(5-(3-chlorophenyl)-1,3,4-oxadiazol-2-yl)-4-((trifluoromethyl)thio)benzenesulfonamide (HSGN-256):

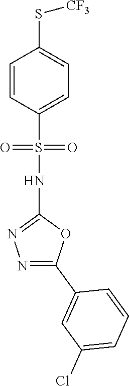 [SMILES: O=S(=O)(Nc1nnc(-c2cccc(Cl)c2)o1)c1ccc(SC(F)(F)F)cc1]

¹H NMR (500 MHz, DMSO-d₆) δ 7.71 (d, J=8.3 Hz, 4H), 7.66 (d, J=8.3 Hz, 4H).

### N-(5-(4-(trifluoromethyl)phenyl)-1,2,4-oxadiazol-3-yl)-4-((trifluoromethyl)thio)benzamide (HSGN-257):

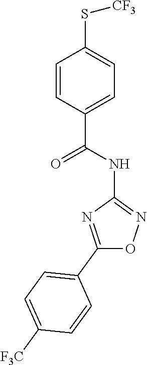 [SMILES: O=C(Nc1noc(-c2ccc(C(F)(F)F)cc2)n1)c1ccc(SC(F)(F)F)cc1]

¹H NMR (500 MHz, DMSO-d₆) δ 7.97 (d, J=8.4 Hz, 2H), 7.70 (d, J=8.4 Hz, 2H), 7.52 (ddd, J=8.1, 6.9, 0.9 Hz, 2H), 7.40 (ddd, J=8.1, 6.9, 1.1 Hz, 2H).

### N-(5-(5-methylthiophen-2-yl)-1,3,4-oxadiazol-2-yl)-4-((trifluoromethyl)thio)benzamide (HSGN-258):

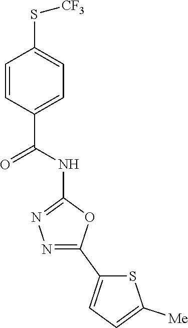 [SMILES: Cc1ccc(-c2nnc(NC(=O)c3ccc(SC(F)(F)F)cc3)o2)s1]

¹H NMR (500 MHz, DMSO-d₆) δ 8.11 (d, J=8.0 Hz, 2H), 7.89 (d, J=8.0 Hz, 2H), 7.56 (d, J=3.7 Hz, 1H), 6.99 (dd, J=3.7, 1.2 Hz, 1H), 2.54 (d, J=1.1 Hz, 3H). HRMS (ESI) m/z calcd for C₁₅H₁₁F₃N₃O₂S₂ [M+H]⁺386.0245, found 386.0246.

### N-(5-(3-chlorophenyl)-1,3,4-oxadiazol-2-yl)-4-(pentafluoro-k6-sulfaneyl)benzamide (HSGN-259):

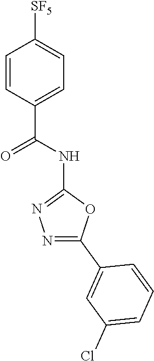

¹H NMR (500 MHz, DMSO-d₆) δ 8.22 (d, J=8.5 Hz, 2H), 8.11 (d, J=8.6 Hz, 2H), 7.91 (d, J=8.2 Hz, 2H), 7.72-7.68 (m, 1H), 7.64 (t, J=7.8 Hz, 1H). HRMS (ESI) m/z calcd for C₁₆H₁₀ClF₅N₃O₂S [M+H]⁺426.0102, found 426.0100.

### N-(5-(3-chlorophenyl)-1,3,4-oxadiazol-2-yl)-3-nitro-4-(trifluoromethoxy)benzamide (HSGN-262):

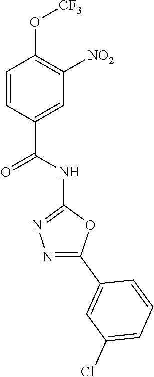 [SMILES: O=C(Nc1nnc(-c2cccc(Cl)c2)o1)c1ccc(OC(F)(F)F)c([N+](=O)[O-])c1]

¹H NMR (500 MHz, DMSO-d₆) δ 7.90 (d, J=8.6 Hz, 1H), 7.77-7.70 (m, 2H), 7.55 (d, J=6.7 Hz, 2H), 7.33 (s, 2H).

### N-(5-(3,5-dichloro-4-methoxyphenyl)-1,3,4-oxadiazol-2-yl)-4-((trifluoromethyl)thio)benzamide (HSGN-265):

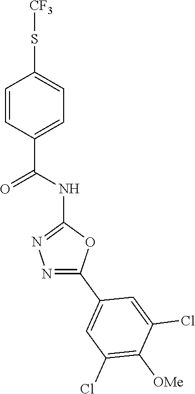 [SMILES: COc1c(Cl)cc(-c2nnc(NC(=O)c3ccc(SC(F)(F)F)cc3)o2)cc1Cl]

¹H NMR (500 MHz, DMSO-d₆) δ 8.17 (d, J=7.5 Hz, 2H), 7.92 (s, 1H), 7.82 (d, J=7.8 Hz, 1H), 3.89 (s, 2H).

### N-(5-(3-chloro-5-methoxyphenyl)-1,3,4-oxadiazol-2-yl)-4-((trifluoromethyl)thio)benzamide (HSGN-266):

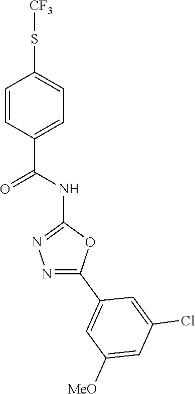 [SMILES: COc1cc(Cl)cc(-c2nnc(NC(=O)c3ccc(SC(F)(F)F)cc3)o2)c1]

¹H NMR (500 MHz, DMSO-d₆) δ 8.13 (d, J=8.0 Hz, 2H), 7.90 (d, J=8.0 Hz, 2H), 7.67 (d, J=1.8 Hz, 1H), 7.50 (d, J=1.8 Hz, 1H), 4.00 (s, 3H).

### N-(5-phenyl-1,3,4-oxadiazol-2-yl)-4-((trifluoromethyl)thio)benzamide (HSGN-280)

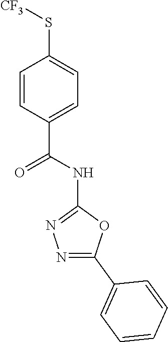 [SMILES: O=C(Nc1nnc(-c2ccccc2)o1)c1ccc(SC(F)(F)F)cc1]

¹H NMR (500 MHz, DMSO-d₆) δ 8.09 (d, J=8.0 Hz, 2H), 7.92 (d, J=8.0 Hz, 2H), 7.99-7.93 (m, 2H), 7.61 (d, J=7.0 Hz, 3H).

### N-(5-(4-fluorophenyl)-1,3,4-oxadiazol-2-yl)-4-((trifluoromethyl)thio)benzamide (HSGN-281)

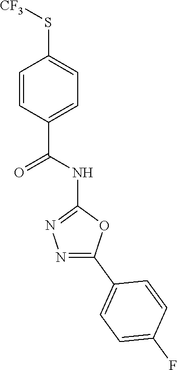 [SMILES: O=C(Nc1nnc(-c2ccc(F)cc2)o1)c1ccc(SC(F)(F)F)cc1]

¹H NMR (500 MHz, DMSO-d₆) δ 8.11 (d, J=8.0 Hz, 2H), 7.90 (d, J=8.1 Hz, 2H), 7.87 (d, J=8.2 Hz, 2H), 7.45 (t, J=8.7 Hz, 2H).

### N-(5-(4-chlorophenyl)-1,3,4-oxadiazol-2-yl)-4-((trifluoromethyl)thio)benzamide (HSGN-282)

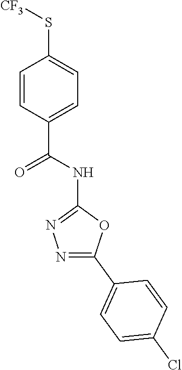 [SMILES: O=C(Nc1nnc(-c2ccc(Cl)cc2)o1)c1ccc(SC(F)(F)F)cc1]

¹H NMR (500 MHz, DMSO-d₆) δ 8.10 (d, J=8.0 Hz, 2H), 8.04-7.96 (m, 2H), 7.88 (d, J=8.1 Hz, 2H), 7.57-7.47 (m, 2H).

### N-(5-(3-cyanophenyl)-1,3,4-oxadiazol-2-yl)-4-((trifluoromethyl)thio)benzamide (HSGN-283)

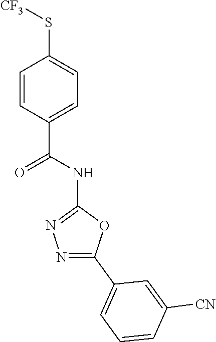 [SMILES: N#Cc1cccc(-c2nnc(NC(=O)c3ccc(SC(F)(F)F)cc3)o2)c1]

¹H NMR (500 MHz, DMSO-d₆) δ 8.24 (d, J=8.0 Hz, 1H), 8.13 (d, J=8.0 Hz, 2H), 8.02 (d, J=7.8 Hz, 1H), 7.82-7.79 (m, 3H), 7.76 (d, J=7.8 Hz, 1H).

### N-(5-(4-fluorophenyl)thiazol-2-yl)-4-(pentafluoro-k⁶-sulfaneyl)benzamide (HSGN-285):

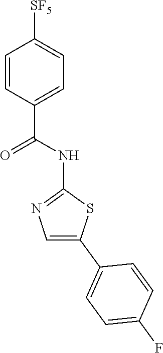

¹H NMR (500 MHz, DMSO-d₆) δ 8.23 (d, J=8.5 Hz, 2H), 8.09 (d, J=8.9 Hz, 2H), 7.75 (d, J=8.2 Hz, 2H), 7.51 (s, 1H), 7.36 (t, J=8.7 Hz, 2H).

### N-(5-(3-chlorophenyl)-1,3,4-oxadiazol-2-yl)-3-((trifluoromethyl)thio)benzamide (HSGN-2111)

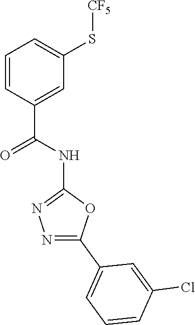

¹H NMR (500 MHz, DMSO-d₆) δ 8.10-8.02 (m, 2H), 7.89-7.75 (m, 2H), 7.52-7.43 (m, 4H).

### N-(5-(3-chlorophenyl)-1,3,4-oxadiazol-2-yl)-3-(pentafluoro-λ⁶-sulfaneyl)benzamide (HSGN-2112):

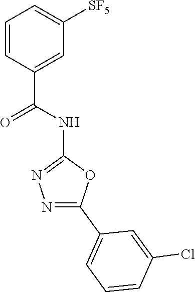

¹H NMR (500 MHz, DMSO-d₆) δ 8.42-8.28 (m, 2H), 8.10-7.95 (m, 3H), 7.55-7.50 (m, 3H).

### N-(5-(5-chlorothiophen-2-yl)-1,3,4-oxadiazol-2-yl)-4-(pentafluoro-λ⁶-sulfaneyl)benzamide (HSGN-2113):

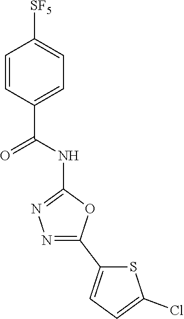

¹H NMR (500 MHz, DMSO-d₆) δ 8.22 (d, J=8.5 Hz, 2H), 8.11 (d, J=8.9 Hz, 2H), 7.61 (d, J=4.0 Hz, 1H), 7.29 (d, J=4.1 Hz, 1H).

### N-(5-(2-fluorophenyl)-1,3,4-oxadiazol-2-yl)-4-((trifluoromethyl)thio)benzamide (HSGN-2114):

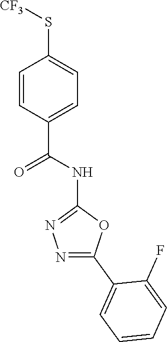 [SMILES: O=C(Nc1nnc(-c2ccccc2F)o1)c1ccc(SC(F)(F)F)cc1]

¹H NMR (500 MHz, DMSO-d₆) δ 8.13 (d, J=8.0 Hz, 2H), 7.98 (td, J=7.6, 1.8 Hz, 1H), 7.92 (d, J=8.1 Hz, 2H), 7.67 (dddd, J=8.7, 7.1, 5.1, 1.8 Hz, 1H), 7.55-7.48 (m, 2H).

### N-(5-(2-chlorophenyl)-1,3,4-oxadiazol-2-yl)-4-((trifluoromethyl)thio)benzamide (HSGN-2115):

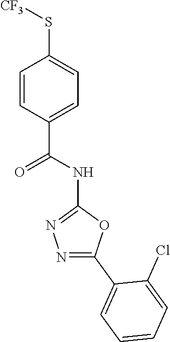 [SMILES: O=C(Nc1nnc(-c2ccccc2Cl)o1)c1ccc(SC(F)(F)F)cc1]

¹H NMR (500 MHz, DMSO-d₆) δ 8.11 (d, J=8.0 Hz, 2H), 8.00 (dd, J=7.8, 1.7 Hz, 1H), 7.88 (d, J=8.1 Hz, 2H), 7.71 (dd, J=8.1, 1.2 Hz, 1H), 7.61 (td, J=7.7, 1.8 Hz, 1H), 7.54 (td, J=7.6, 1.3 Hz, 1H).

4-(pentafluoro-λ⁶-sulfaneyl)-N-(1-phenyl-1H-pyrazol-4-yl)benzamide (HSGN2135):

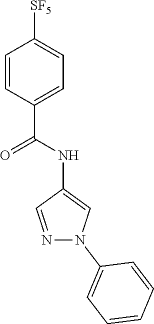

¹H NMR (500 MHz, DMSO-d₆) δ 8.22 (d, J=8.5 Hz, 2H), 8.11 (d, J=8.9 Hz, 2H), 8.03 (s, 1H), 7.72 (s, 1H), 7.68-7.62 (m, 2H), 7.53 (d, J=7.0 Hz, 3H).

### N-(2-benzyl-2H-tetrazol-5-yl)-4-(pentafluoro-λ⁶-sulfaneyl)benzamide (HSGN-2136):

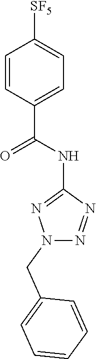

¹H NMR (500 MHz, DMSO-d₆) δ 8.24 (d, J=8.5 Hz, 2H), 8.12 (d, J=8.9 Hz, 2H), 7.34-7.29 (m, 3H), 7.21 (m, 2H), 4.97 (s, 2H).

### N-(5-methyl-1,3,4-oxadiazol-2-yl)-4-(pentafluoro-λ⁶-sulfaneyl)benzamide (HSGN-2140):

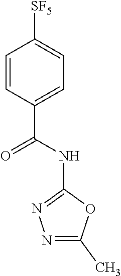

¹H NMR (500 MHz, DMSO-d₆) δ 8.26 (d, J=8.5 Hz, 2H), 8.10 (d, J=8.9 Hz, 2H), 2.59 (s, 1H).

### 4-(pentafluoro-λ⁶-sulfaneyl)-N-(5-(trifluoromethyl)-1,3,4-oxadiazol-2-yl)benzamide (HSGN-2141):

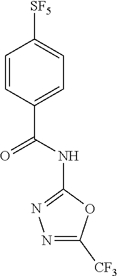

¹H NMR (500 MHz, DMSO-d₆) δ 8.23 (d, J=8.5 Hz, 2H), 8.07 (d, J=8.9 Hz, 2H).

### 4-(pentafluoro-λ⁶-sulfaneyl)-N-(5-propyl-1,3,4-oxadiazol-2-yl)benzamide (HSGN-2142):

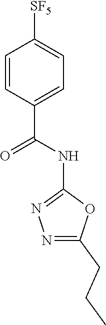

¹H NMR (500 MHz, DMSO-d₆) δ 8.24 (d, J=8.5 Hz, 2H), 8.10 (d, J=8.9 Hz, 2H), 2.59 (d, J=7.5 Hz, 2H), 1.68 (m, 2H), 1.01 (s, J=8.0 Hz, 3H).

### N-(5-cyclopentyl-1,3,4-oxadiazol-2-yl)-4-(pentafluoro-16-sulfaneyl)benzamide (HSGN-2143):

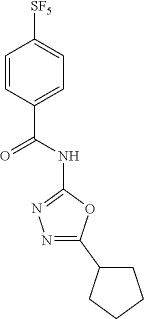

¹H NMR (500 MHz, DMSO-d₆) δ 8.18 (d, J=8.5 Hz, 2H), 8.09 (d, J=8.9 Hz, 2H), 2.85-2.79 (m, 1H), 1.94-1.68 (m, 4H), 1.65-1.59 (m, 4H).

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

It is intended that that the scope of the present methods and compositions be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

### REFERENCES CITED

1. Gould, I. M.; Bal, A. M., New antibiotic agents in the pipeline and how they can help overcome microbial resistance. Virulence 2013, 4 (2), 185-91.
2. Wright, G. D., Something old, something new: revisiting natural products in antibiotic drug discovery. Can. J. Microbiol. 2014, 60 (3), 147-54.
3. Ventola, C. L., The antibiotic resistance crisis: part 1: causes and threats. P T 2015, 40 (4), 277-83.
4. Bush, K.; Courvalin, P.; Dantas, G.; et al. Nat. Rev. Microbiol. 2011, 9 (12), 894-6.
5. Frieden, T., Antibiotic Resistance Threats in the United States, 2013. Centers for Disease

Control and Prevention: Atlanta, Ga., USA, 2013; p 114.

1. A compound having a formula (I) 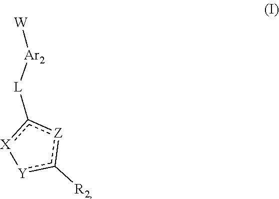
or a pharmaceutically acceptable salt thereof, wherein [symbol]
represents a single or double bond;
X, Y or Z is, independently, CH, O, S, N, or NR, wherein R is H, alkyl, aryl, heteroalkyl, or heteroaryl;
W is —CF₃, —OCF₃, —OCHF₂, —SCF₃, or —SF₅;
L is a linkage selected from the group comprising —NH—, NR—, —NHCO—, —CO—NH—, —NRCO—, —CO—NR—, —CO—, —NH—SO₂—, —NR—SO₂—, —O—, —S—, —S(═O)—, or —S(O₂)—;
R₂ is an optionally substituted aryl, heteroaryl, alkyl (linear, branched or cyclic), heteroalkyl (linear, branched or cyclic), amide, CN, urea, sulfone, sulfoxide, sulfonamide;
and
Ar₂ is an optionally substituted aryl or heteroaryl.

2. The compound according to claim 1, wherein the compound has a formula (II) 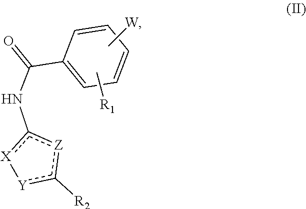
wherein [symbol] represents a single or double bond;
X, Y or Z is, independently, CH, O, S, N, or NR, wherein R is H, alkyl, aryl, heteroalkyl, or heteroaryl;
W is —CF₃, —OCF₃, —OCHF₂, —SCF₃, or —SF₅;
R₁ represents four substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted; or any two adjacent substituents are taken together with the attached carbons form an optionally substituted cyclic or heterocyclic moiety;
and
R₂ is an optionally substituted aryl, heteroaryl, alkyl (linear, branched or cyclic), heteroalkyl (linear, branched or cyclic), amide, CN, urea, sulfone, sulfoxide, sulfonamide.

3. The compound according to claim 1, wherein X and Y are N, and Z is O.

4. The compound according to claim 2, wherein at least one of the four R₁ substituents is hydrogen, halo, alkoxy, or heteroalkoxy.

5-6. (canceled)

7. The compound according to claim 1, wherein W is —SCF₃, —OCF₃, —CF₃ or —SF₅.

8-9. (canceled)

10. The compound according to claim 1, wherein the compound is one of the following: 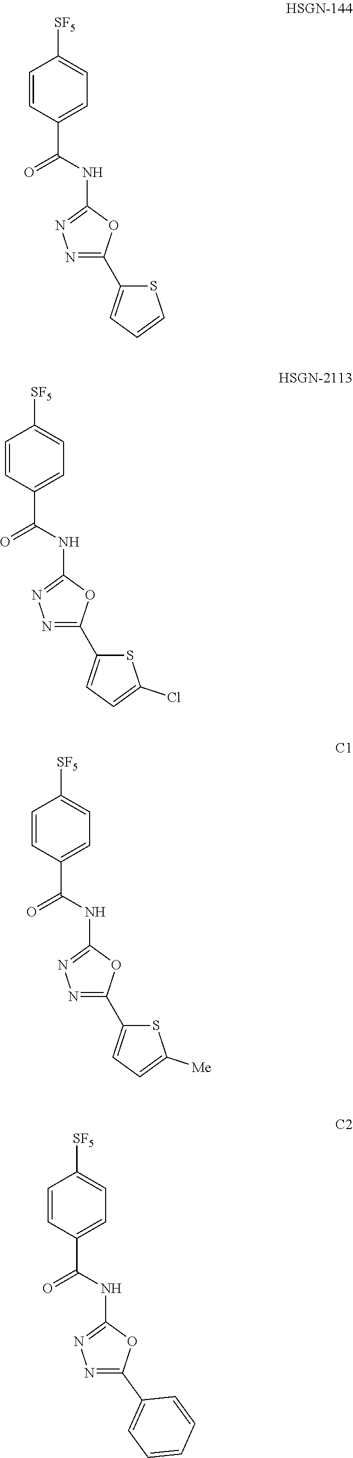 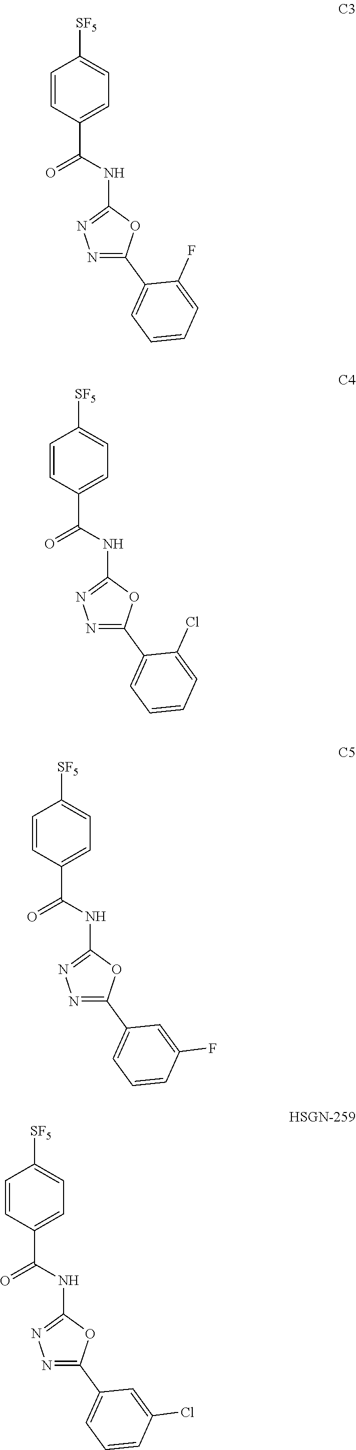 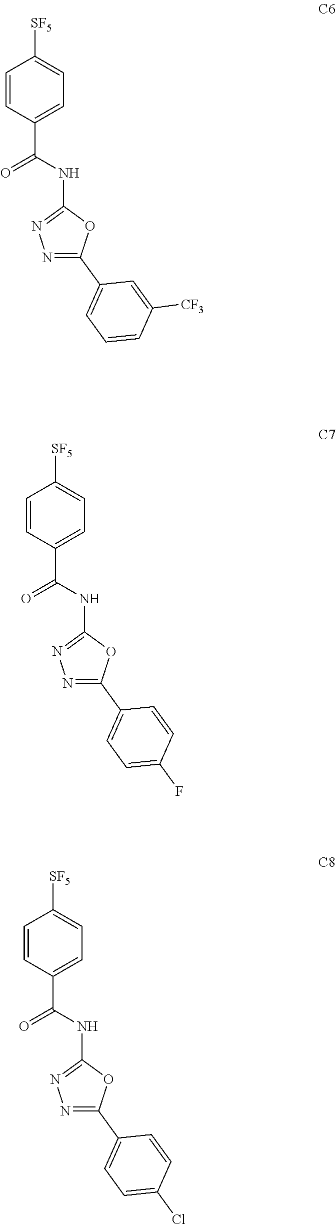 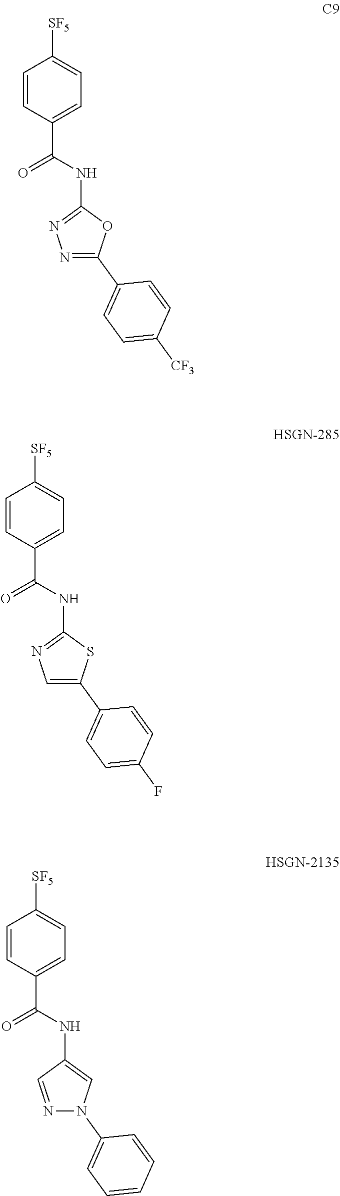 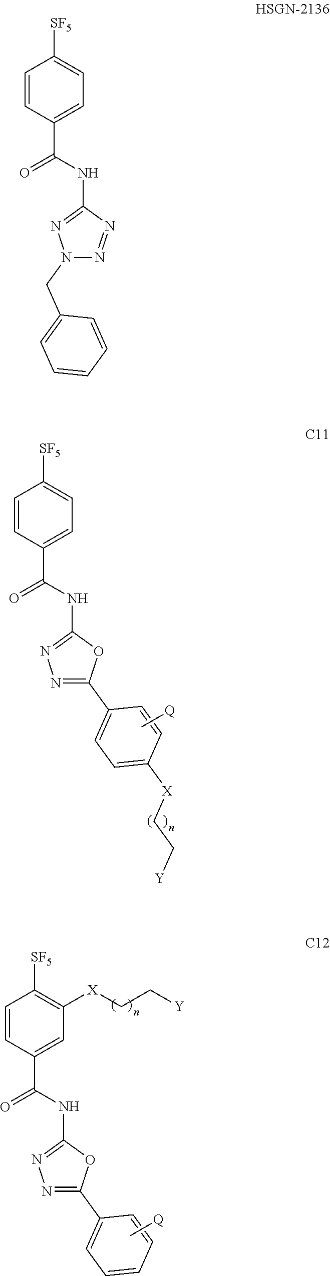 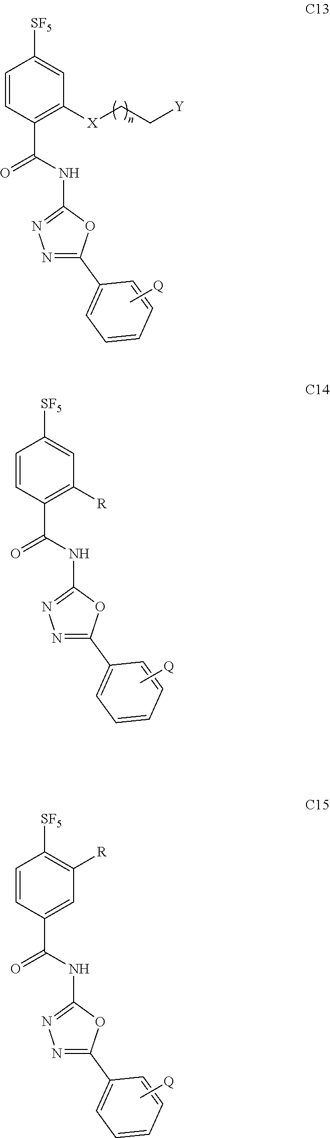 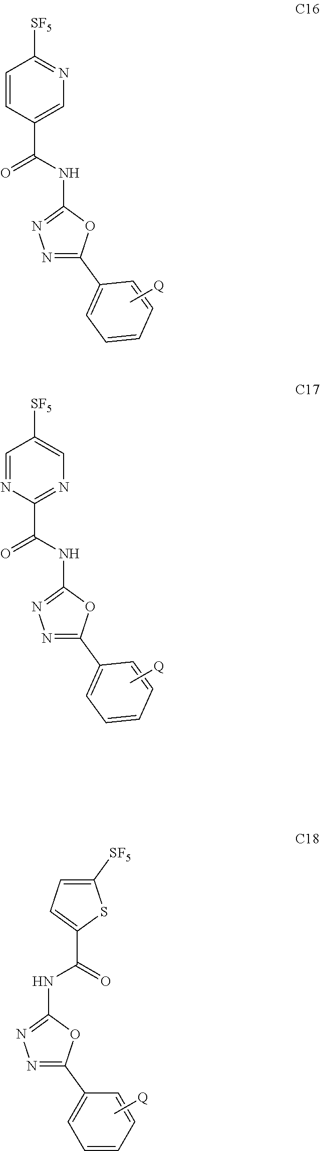 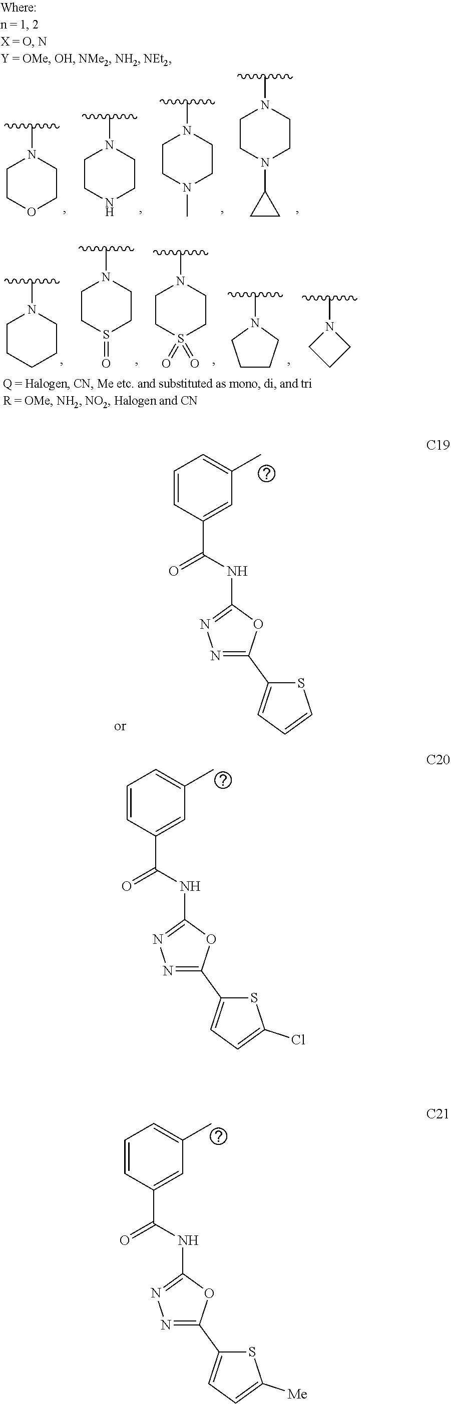 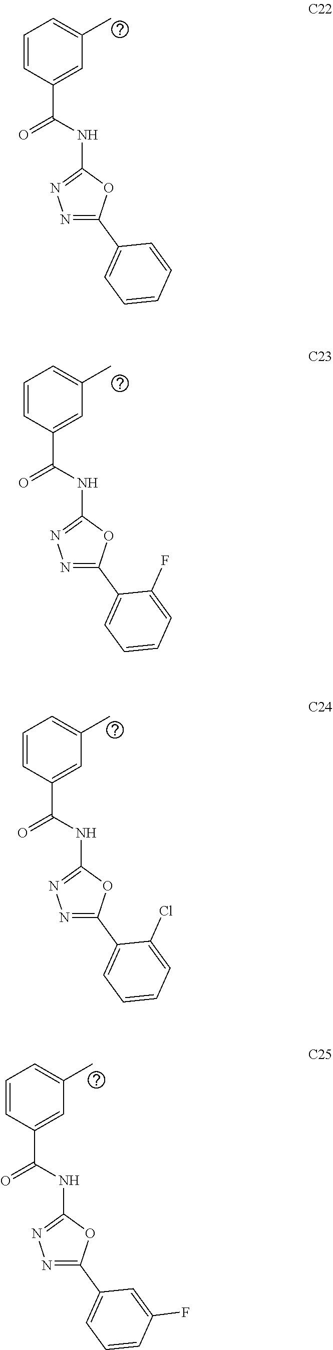 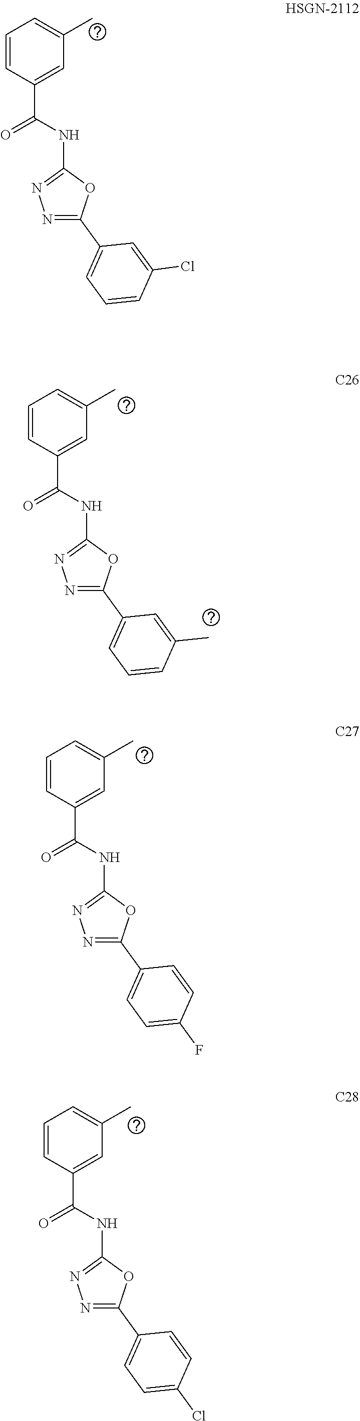 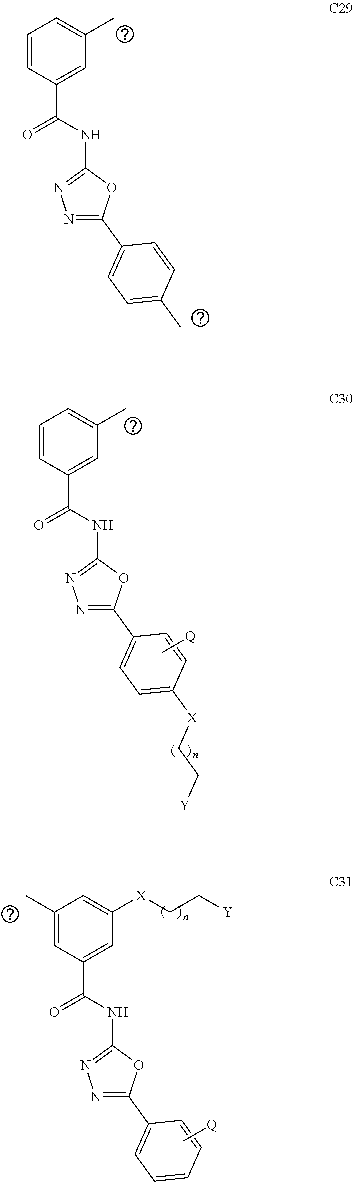 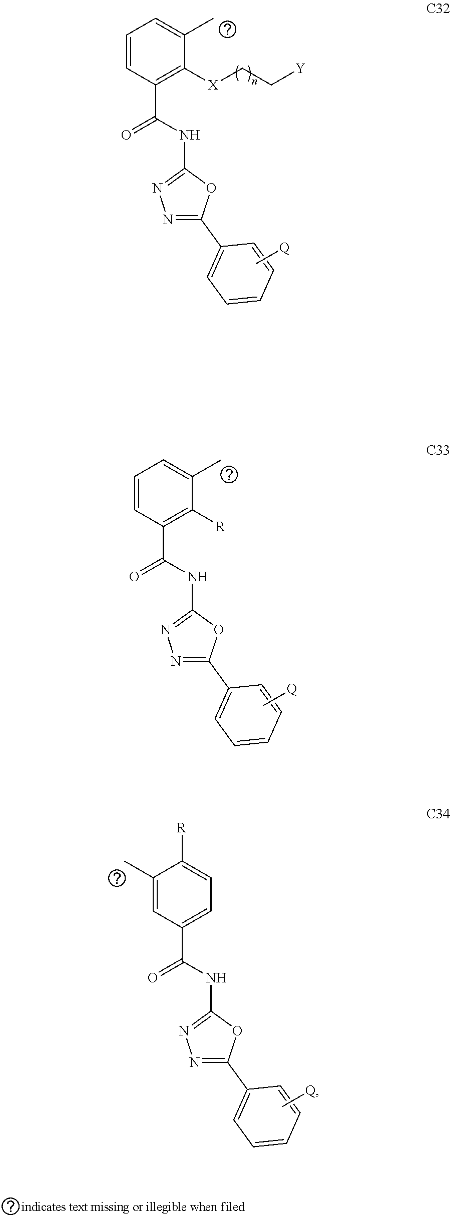
where:
n is 1 or 2,
X is O or N,
Y is OMe, OH, NMe₂, NH₂, NEt₂, 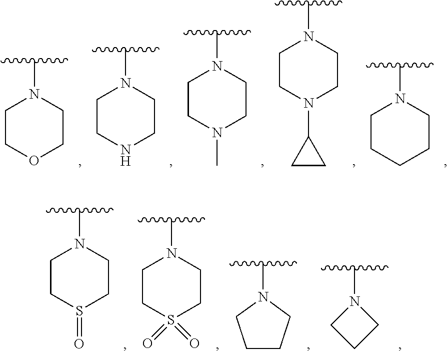
Q is halogen, CN, Me, etc. and substituted as mono, di and tri, and
R is selected from OMe, NH₂, NO₂, halogen, and CN.

11. (canceled)

12. The compound according to claim 1, wherein the compound is one of the following: 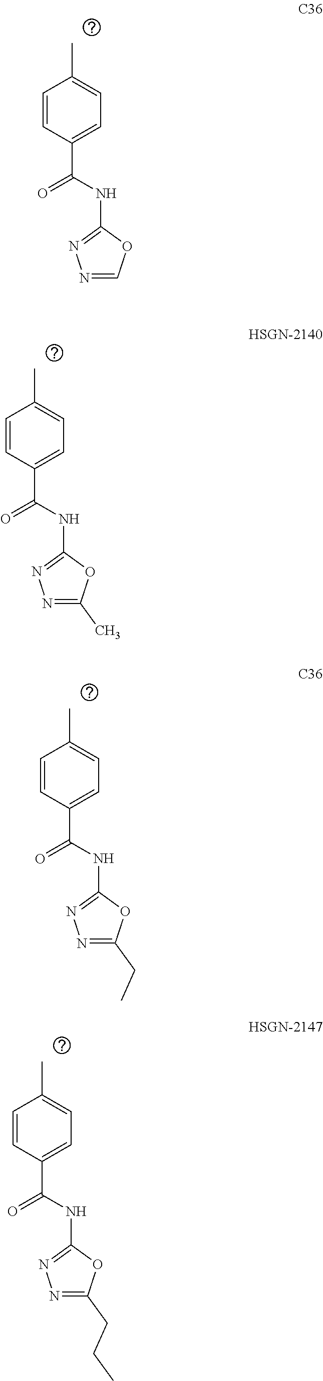 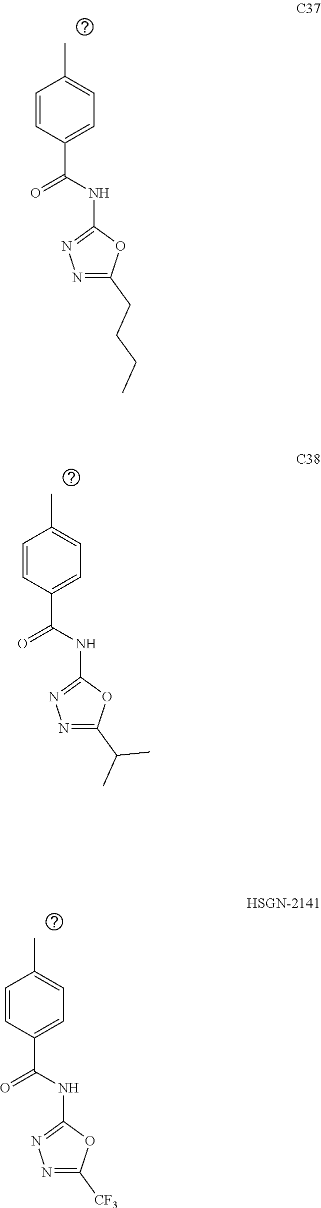 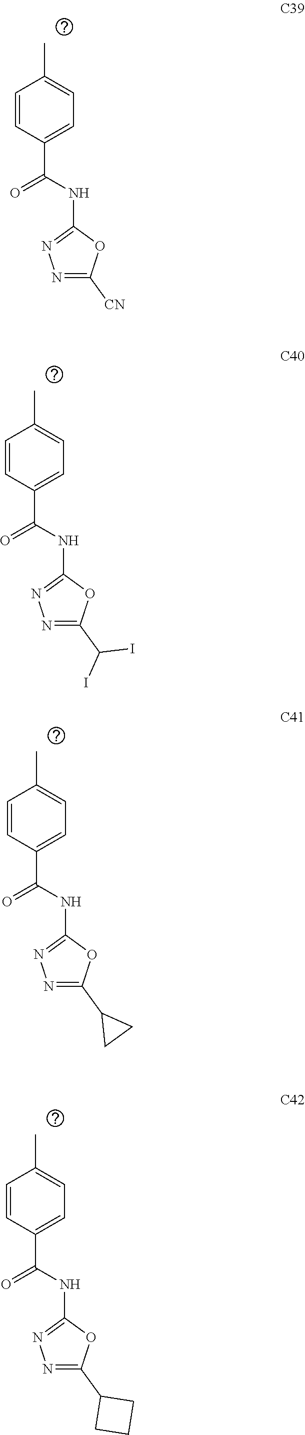 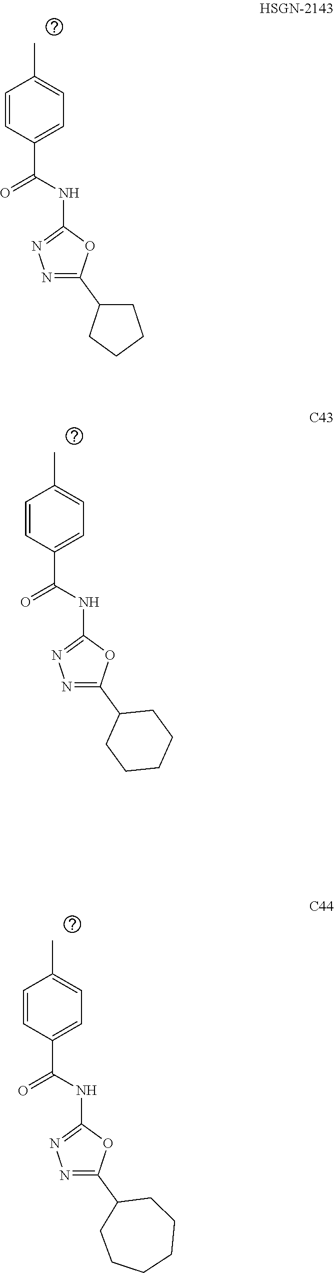 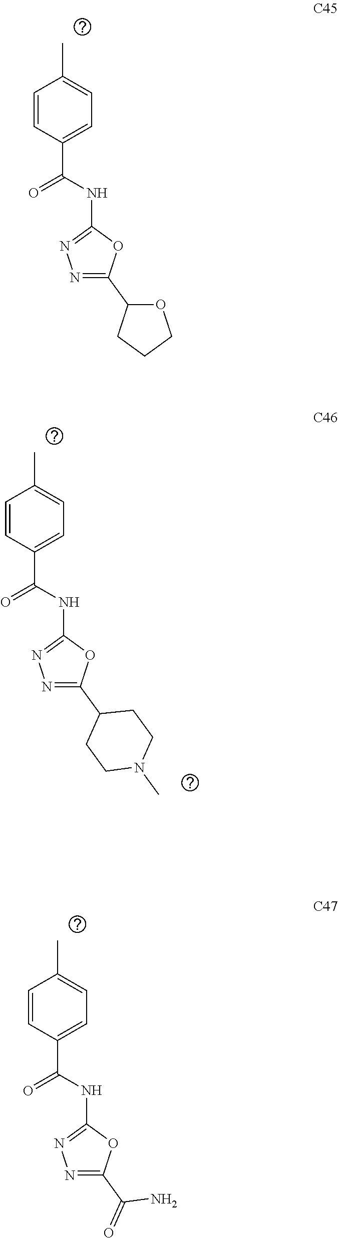 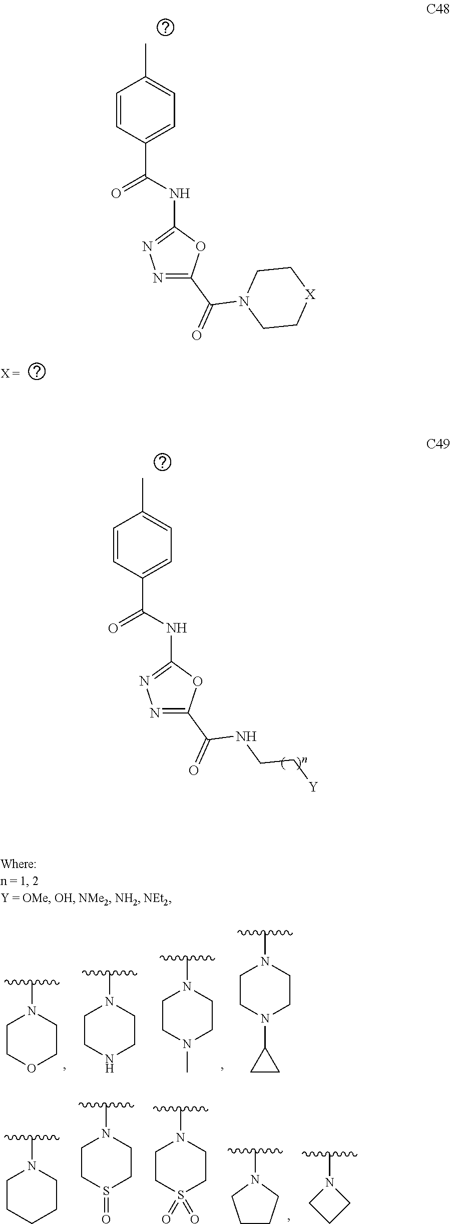 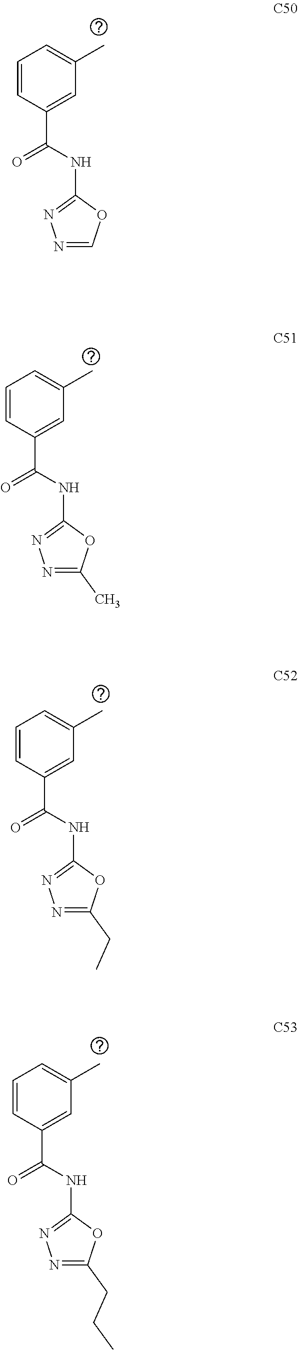 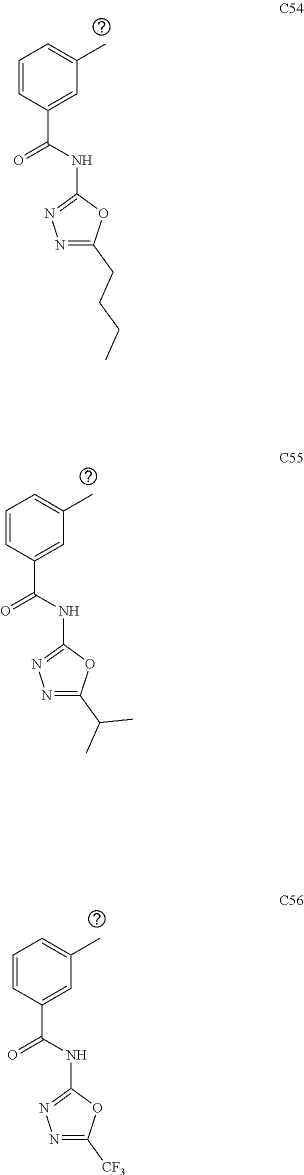 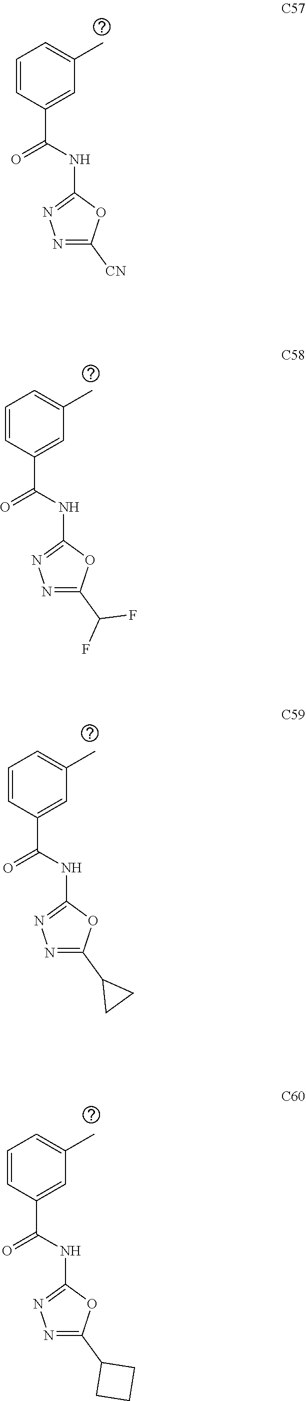 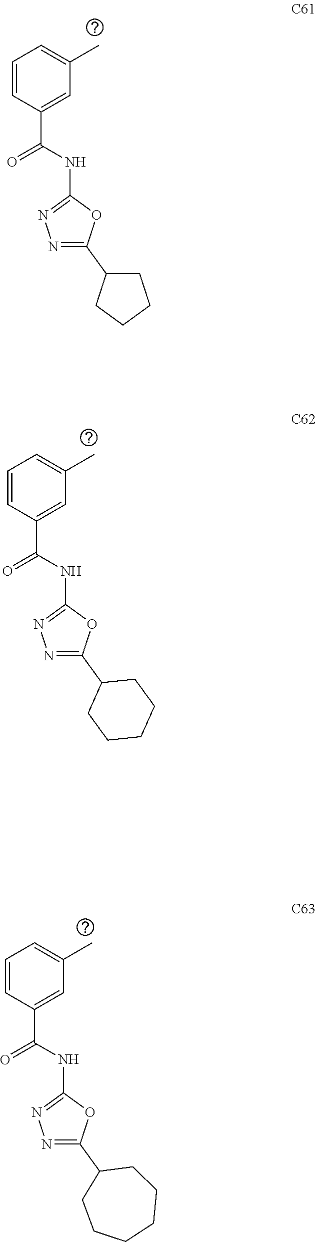 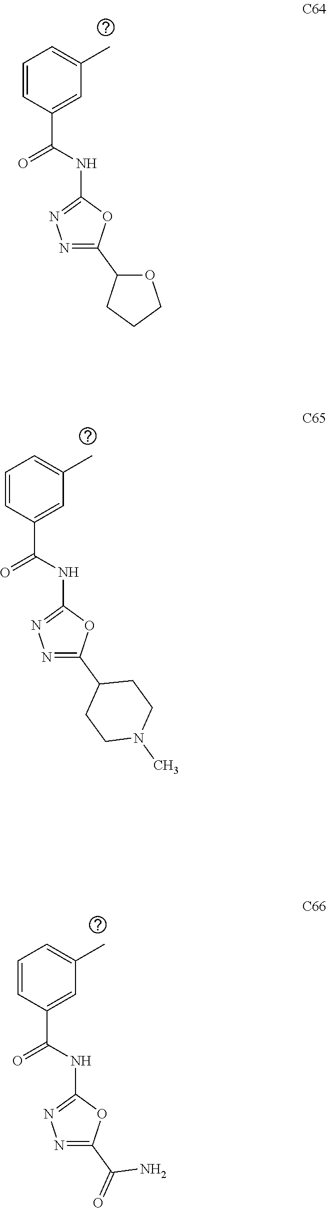 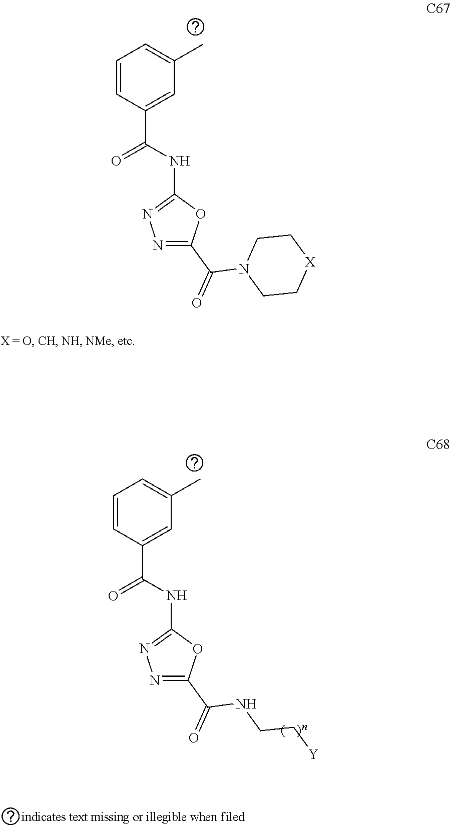
where:
n is 1 or 2, and
Y is OMe, OH, NMe₂, NH₂, NEt₂,

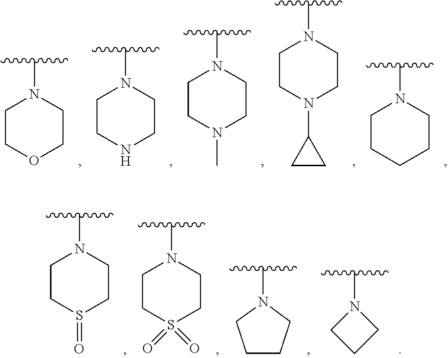

13. (canceled)

14. The compound according to claim 1, wherein the compound is one of the following:

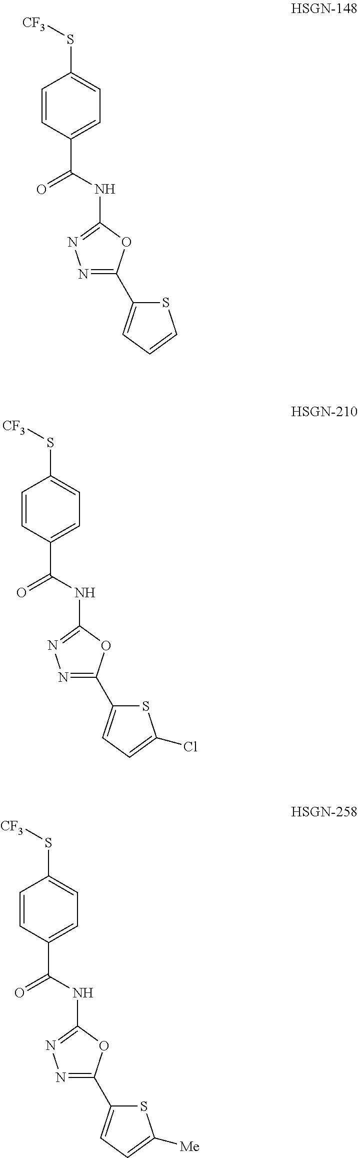 [SMILES: O=C(Nc1nnc(-c2cccs2)o1)c1ccc(SC(F)(F)F)cc1 ; O=C(Nc1nnc(-c2ccc(Cl)s2)o1)c1ccc(SC(F)(F)F)cc1 ; Cc1ccc(-c2nnc(NC(=O)c3ccc(SC(F)(F)F)cc3)o2)s1]

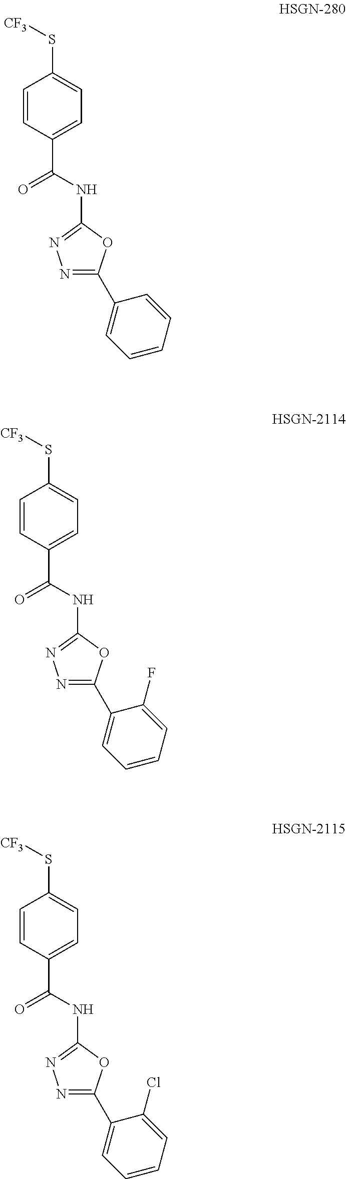 [SMILES: O=C(Nc1nnc(-c2ccccc2)o1)c1ccc(SC(F)(F)F)cc1 ; O=C(Nc1nnc(-c2ccccc2F)o1)c1ccc(SC(F)(F)F)cc1 ; O=C(Nc1nnc(-c2ccccc2Cl)o1)c1ccc(SC(F)(F)F)cc1]

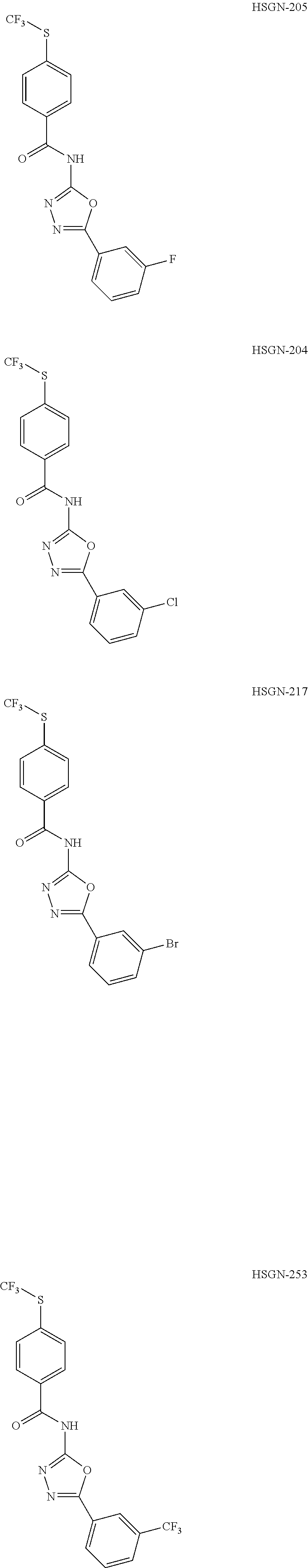 [SMILES: O=C(Nc1nnc(-c2cccc(F)c2)o1)c1ccc(SC(F)(F)F)cc1 ; O=C(Nc1nnc(-c2cccc(Cl)c2)o1)c1ccc(SC(F)(F)F)cc1 ; O=C(Nc1nnc(-c2cccc(Br)c2)o1)c1ccc(SC(F)(F)F)cc1 ; O=C(Nc1nnc(-c2cccc(C(F)(F)F)c2)o1)c1ccc(SC(F)(F)F)cc1]

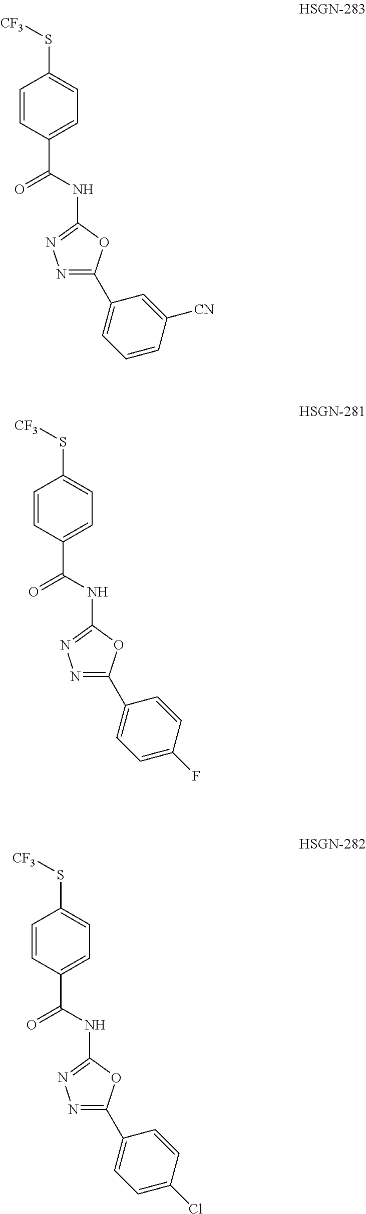 [SMILES: N#Cc1cccc(-c2nnc(NC(=O)c3ccc(SC(F)(F)F)cc3)o2)c1 ; O=C(Nc1nnc(-c2ccc(F)cc2)o1)c1ccc(SC(F)(F)F)cc1 ; O=C(Nc1nnc(-c2ccc(Cl)cc2)o1)c1ccc(SC(F)(F)F)cc1]

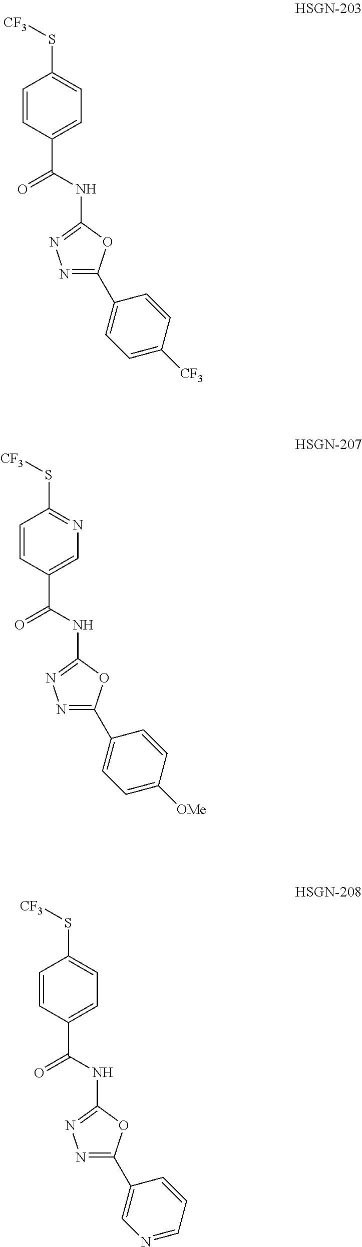 [SMILES: O=C(Nc1nnc(-c2ccc(C(F)(F)F)cc2)o1)c1ccc(SC(F)(F)F)cc1 ; COc1ccc(-c2nnc(NC(=O)c3ccc(SC(F)(F)F)nc3)o2)cc1 ; O=C(Nc1nnc(-c2cccnc2)o1)c1ccc(SC(F)(F)F)cc1]

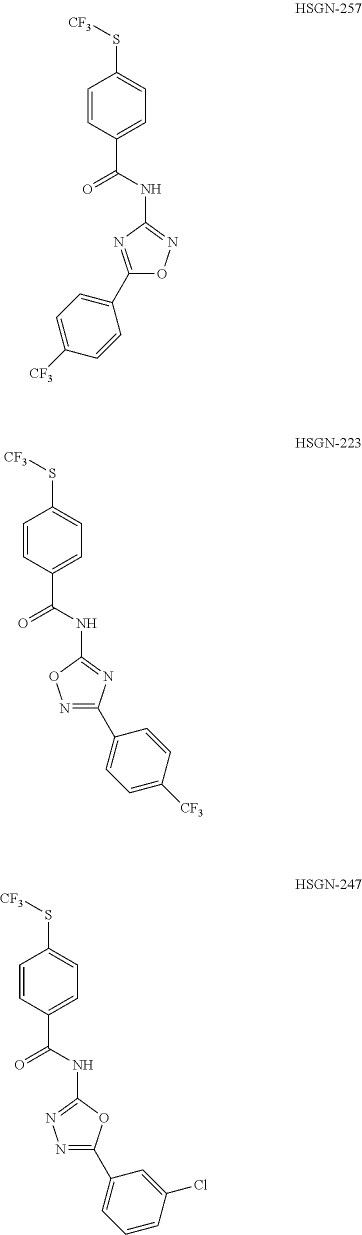 [SMILES: O=C(Nc1noc(-c2ccc(C(F)(F)F)cc2)n1)c1ccc(SC(F)(F)F)cc1 ; O=C(Nc1nc(-c2ccc(C(F)(F)F)cc2)no1)c1ccc(SC(F)(F)F)cc1 ; O=C(Nc1nnc(-c2cccc(Cl)c2)o1)c1ccc(SC(F)(F)F)cc1]

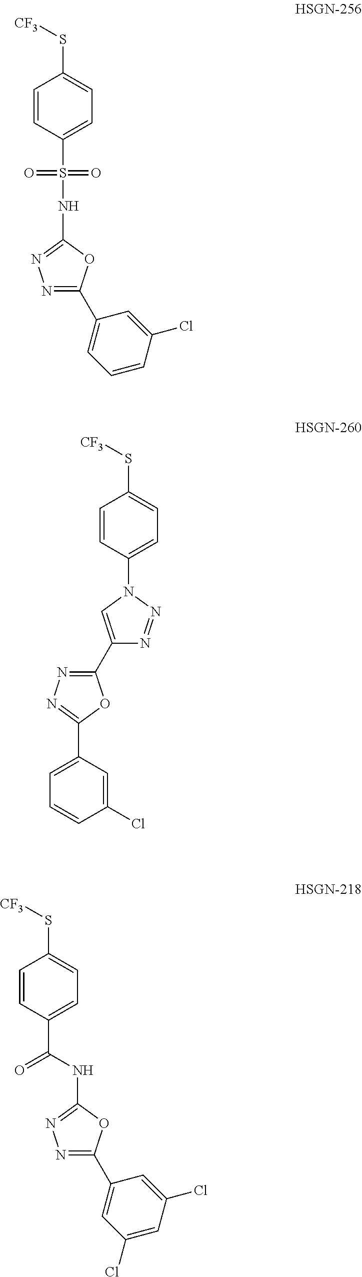 [SMILES: O=S(=O)(Nc1nnc(-c2cccc(Cl)c2)o1)c1ccc(SC(F)(F)F)cc1 ; FC(F)(F)Sc1ccc(-n2cc(-c3nnc(-c4cccc(Cl)c4)o3)nn2)cc1 ; O=C(Nc1nnc(-c2cc(Cl)cc(Cl)c2)o1)c1ccc(SC(F)(F)F)cc1]

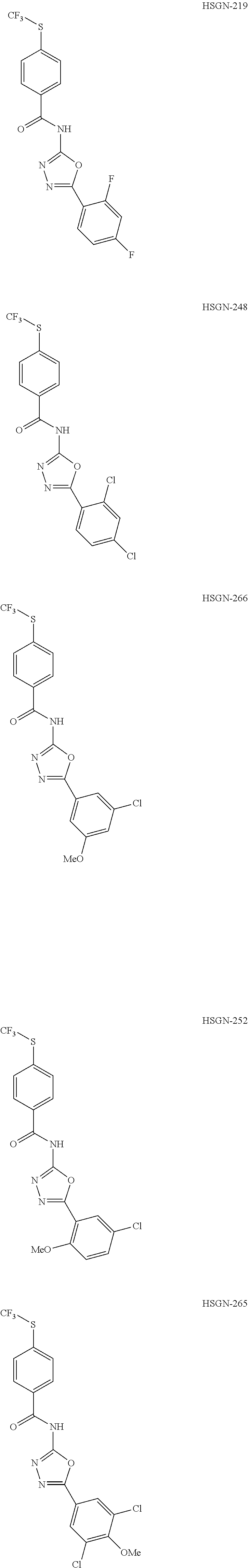 [SMILES: O=C(Nc1nnc(-c2ccc(F)cc2F)o1)c1ccc(SC(F)(F)F)cc1 ; O=C(Nc1nnc(-c2ccc(Cl)cc2Cl)o1)c1ccc(SC(F)(F)F)cc1 ; COc1cc(Cl)cc(-c2nnc(NC(=O)c3ccc(SC(F)(F)F)cc3)o2)c1 ; COc1ccc(Cl)cc1-c1nnc(NC(=O)c2ccc(SC(F)(F)F)cc2)o1 ; COc1c(Cl)cc(-c2nnc(NC(=O)c3ccc(SC(F)(F)F)cc3)o2)cc1Cl]

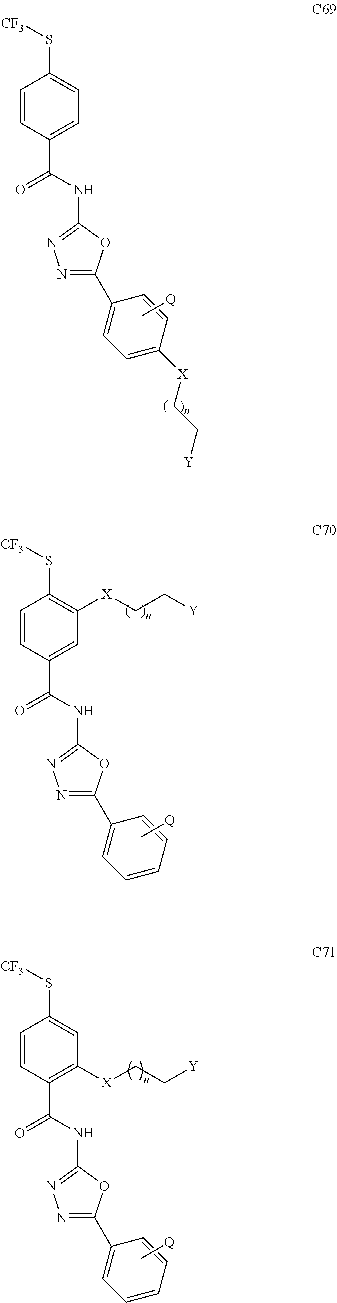

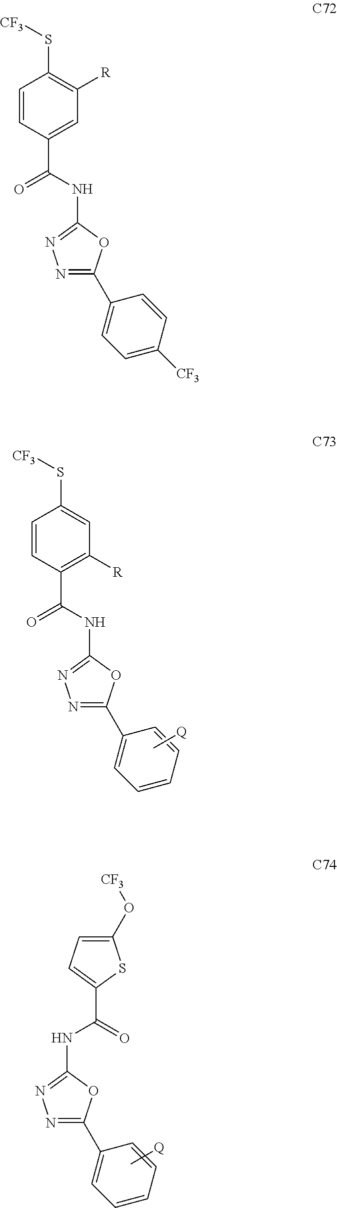

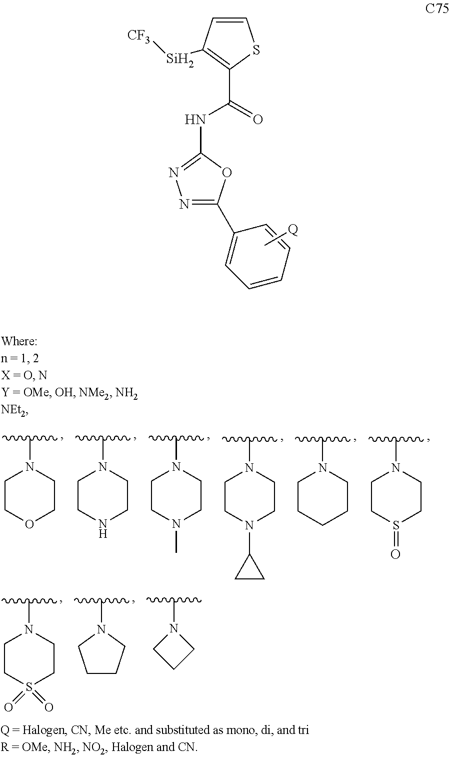

15. The compound according to claim 1, wherein the compound is one of the following: 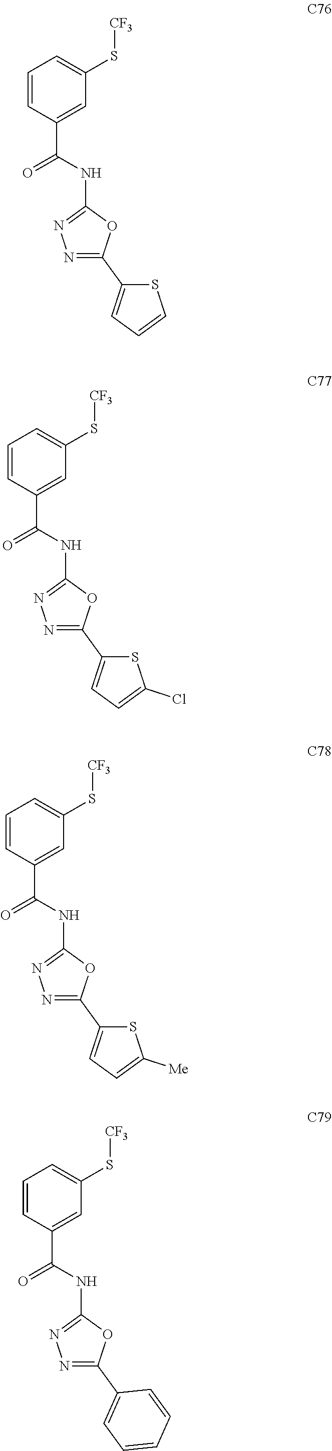 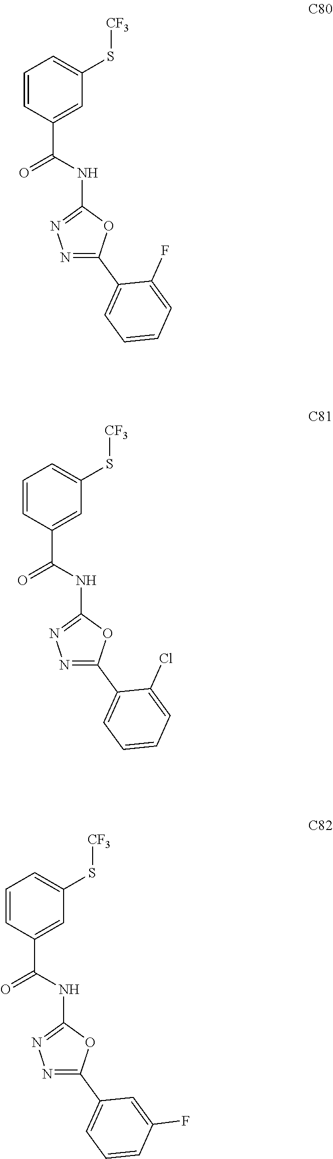 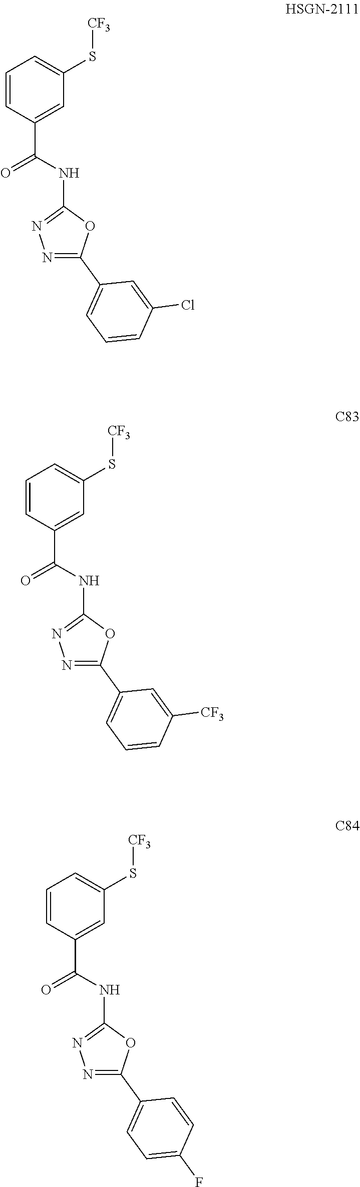 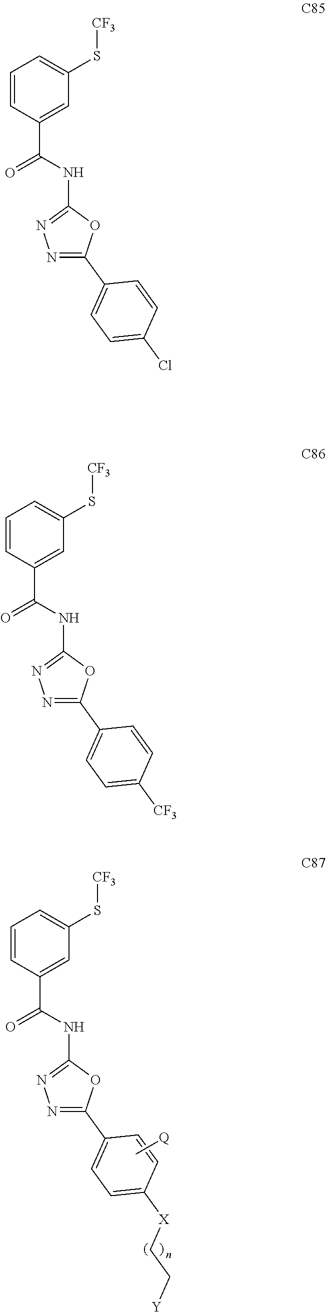 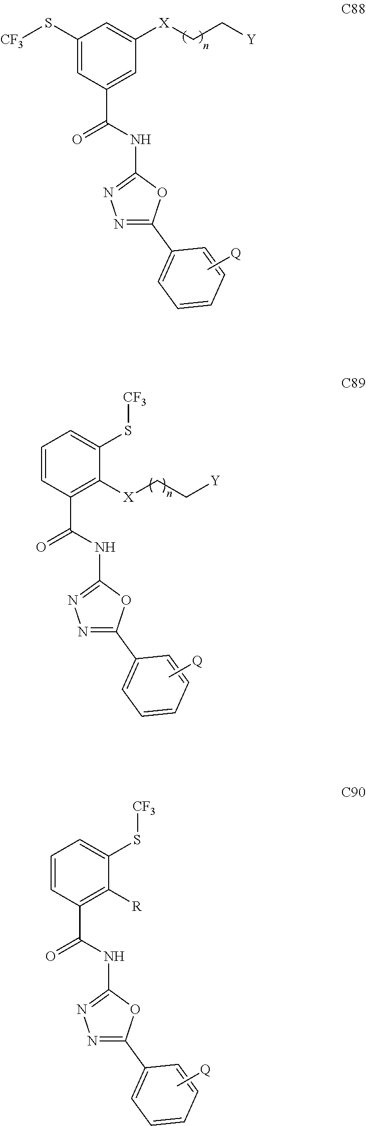 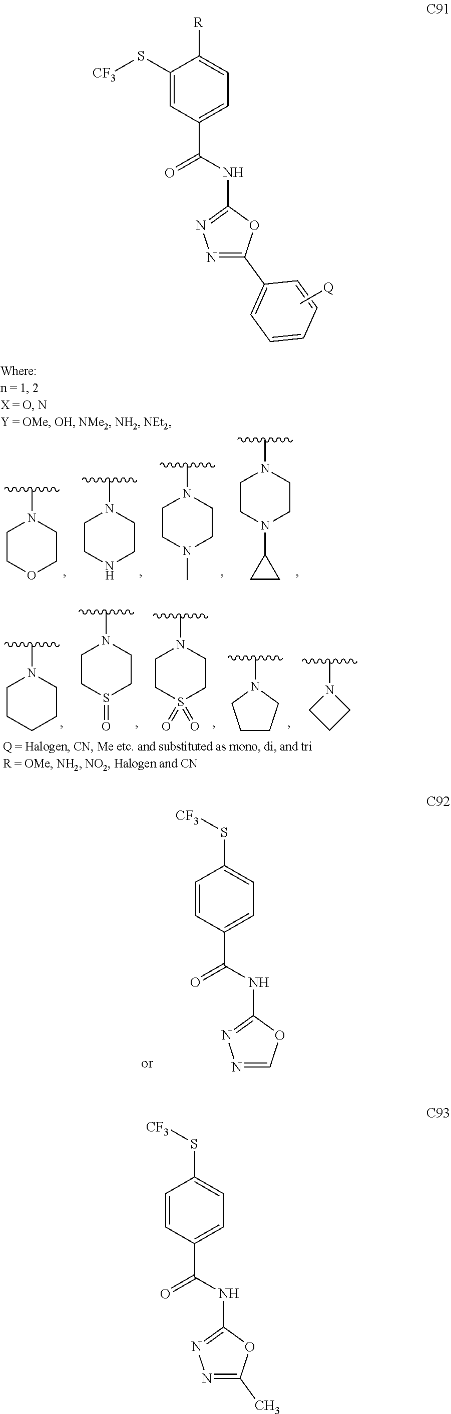 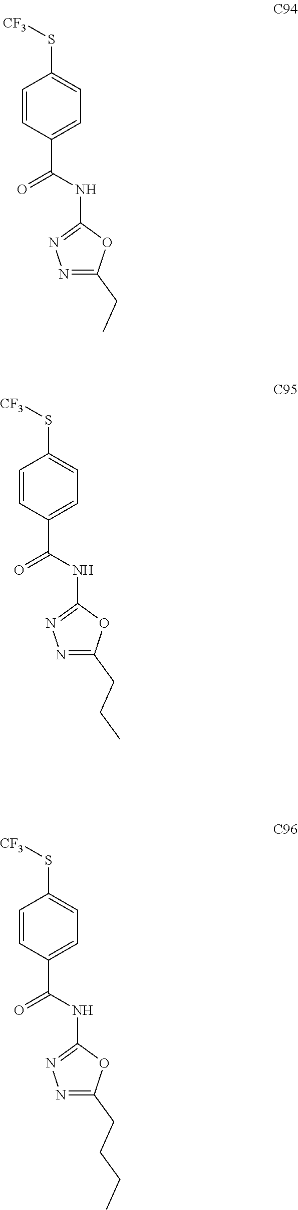 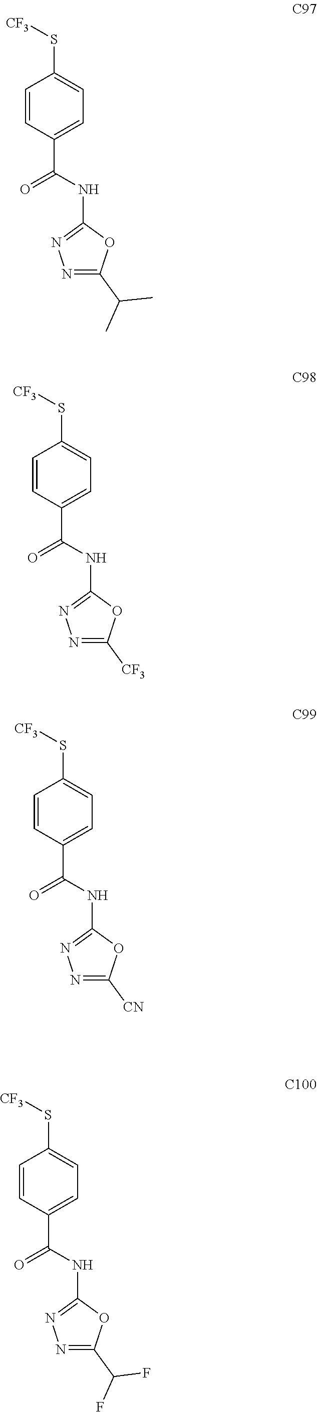 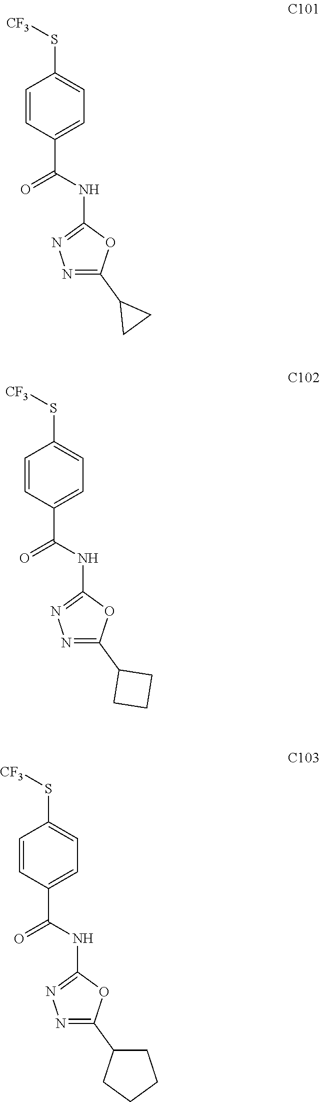 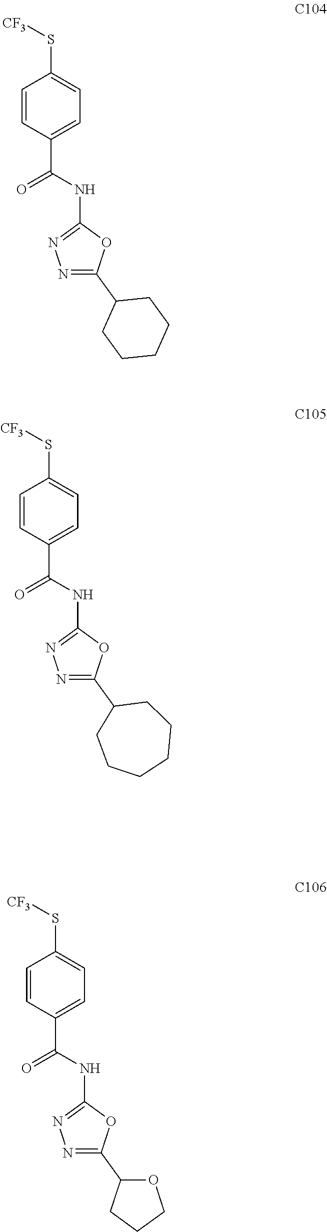 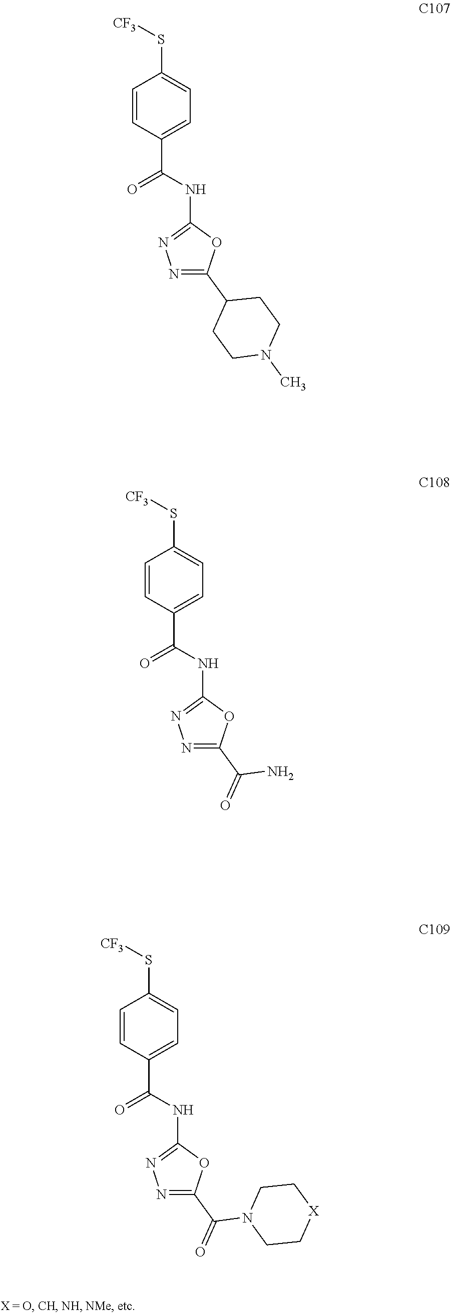 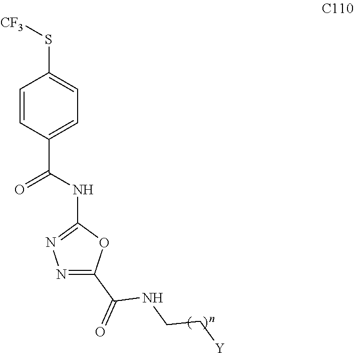
where:
n is 1 or 2,
Y is OMe, OH, NMe₂, NH₂, NEt₂,

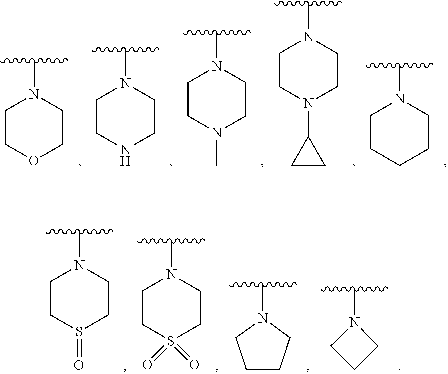

16. (canceled)

17. The compound according to claim 1, wherein the compound is one of the following:

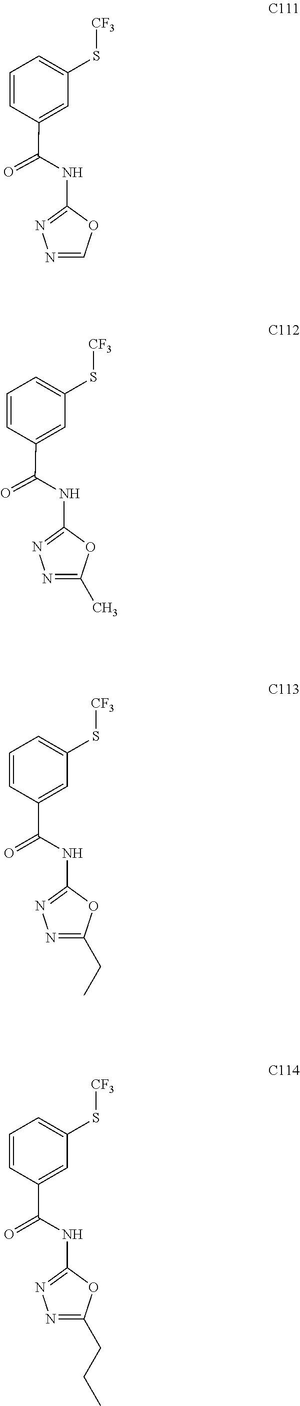 [SMILES: O=C(Nc1nnco1)c1cccc(SC(F)(F)F)c1 ; Cc1nnc(NC(=O)c2cccc(SC(F)(F)F)c2)o1 ; CCc1nnc(NC(=O)c2cccc(SC(F)(F)F)c2)o1 ; CCCc1nnc(NC(=O)c2cccc(SC(F)(F)F)c2)o1]

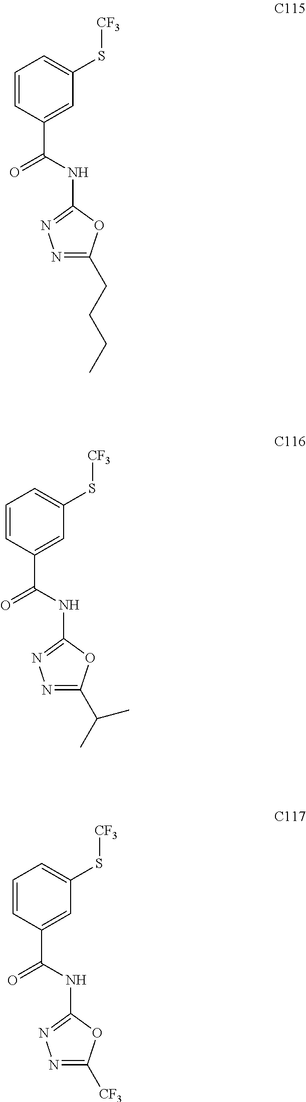 [SMILES: CCCCc1nnc(NC(=O)c2cccc(SC(F)(F)F)c2)o1 ; CC(C)c1nnc(NC(=O)c2cccc(SC(F)(F)F)c2)o1 ; O=C(Nc1nnc(C(F)(F)F)o1)c1cccc(SC(F)(F)F)c1]

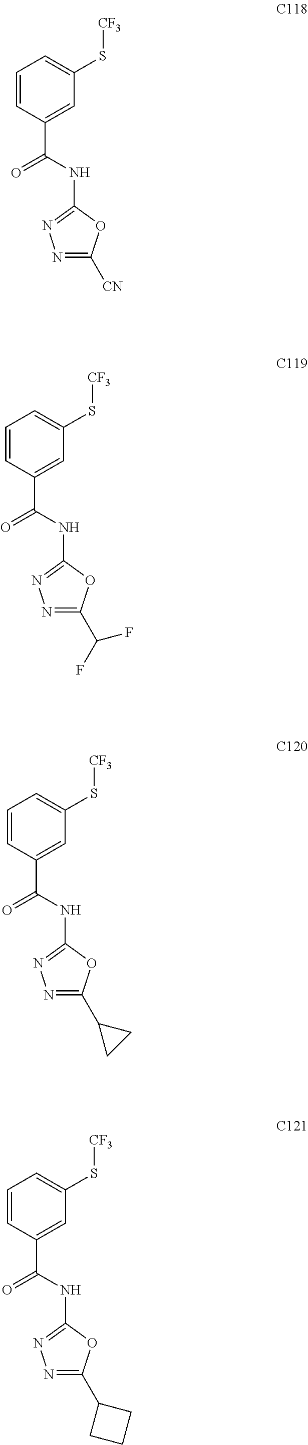 [SMILES: N#Cc1nnc(NC(=O)c2cccc(SC(F)(F)F)c2)o1 ; O=C(Nc1nnc(C(F)F)o1)c1cccc(SC(F)(F)F)c1 ; O=C(Nc1nnc(C2CC2)o1)c1cccc(SC(F)(F)F)c1 ; O=C(Nc1nnc(C2CCC2)o1)c1cccc(SC(F)(F)F)c1]

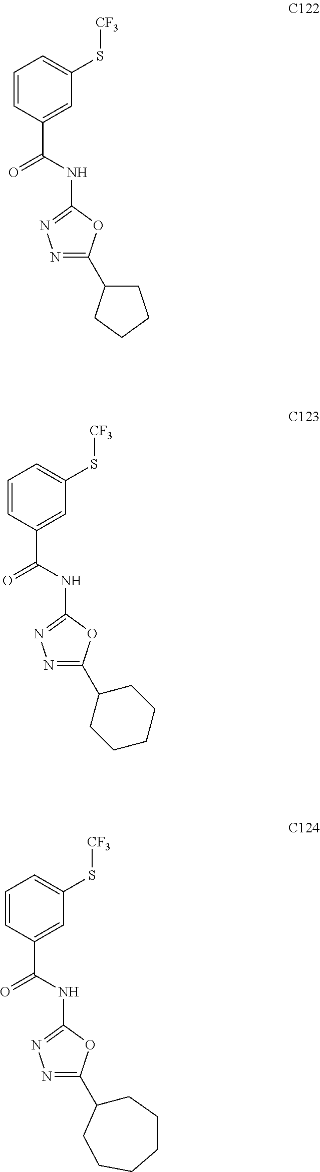 [SMILES: O=C(Nc1nnc(C2CCCC2)o1)c1cccc(SC(F)(F)F)c1 ; O=C(Nc1nnc(C2CCCCC2)o1)c1cccc(SC(F)(F)F)c1 ; O=C(Nc1nnc(C2CCCCCC2)o1)c1cccc(SC(F)(F)F)c1]

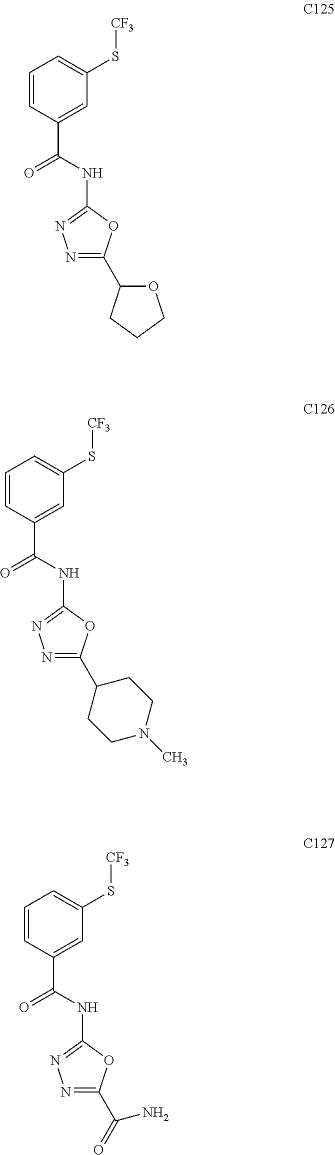 [SMILES: O=C(Nc1nnc(C2CCCO2)o1)c1cccc(SC(F)(F)F)c1 ; CN1CCC(c2nnc(NC(=O)c3cccc(SC(F)(F)F)c3)o2)CC1 ; NC(=O)c1nnc(NC(=O)c2cccc(SC(F)(F)F)c2)o1]

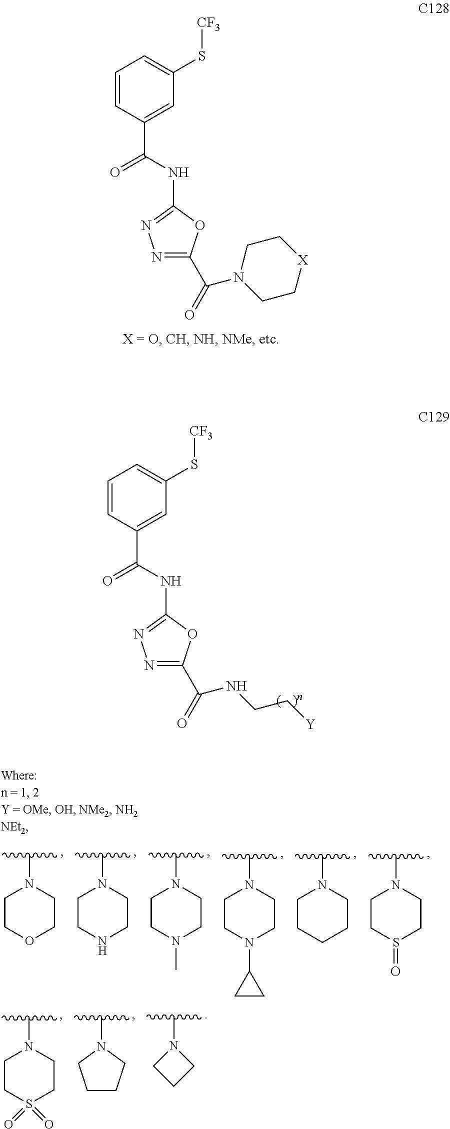

18. The compound according to claim 1, wherein the compound is one of the following:

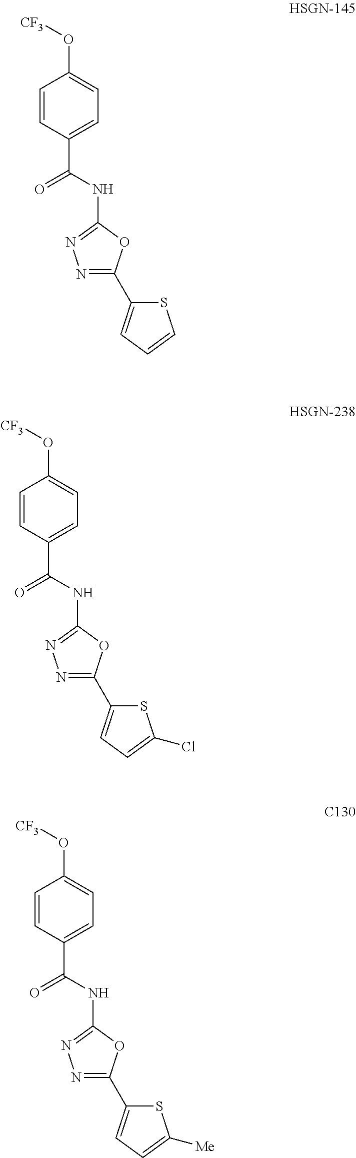 [SMILES: O=C(Nc1nnc(-c2cccs2)o1)c1ccc(OC(F)(F)F)cc1 ; O=C(Nc1nnc(-c2ccc(Cl)s2)o1)c1ccc(OC(F)(F)F)cc1 ; Cc1ccc(-c2nnc(NC(=O)c3ccc(OC(F)(F)F)cc3)o2)s1]

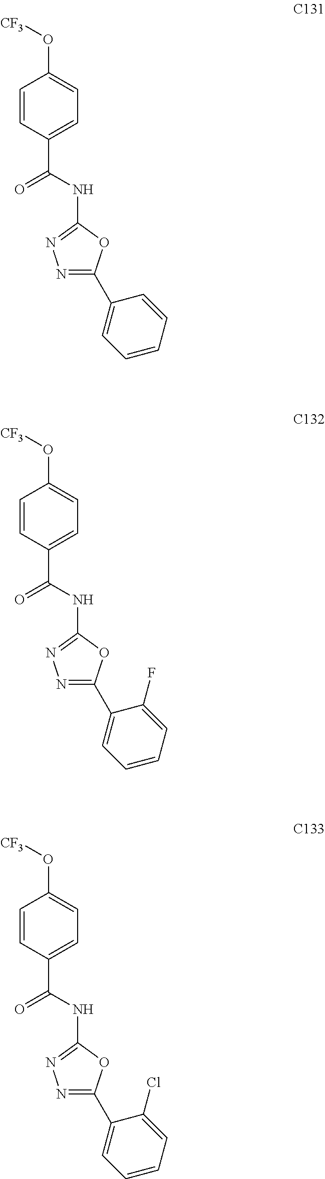 [SMILES: O=C(Nc1nnc(-c2ccccc2)o1)c1ccc(OC(F)(F)F)cc1 ; O=C(Nc1nnc(-c2ccccc2F)o1)c1ccc(OC(F)(F)F)cc1 ; O=C(Nc1nnc(-c2ccccc2Cl)o1)c1ccc(OC(F)(F)F)cc1]

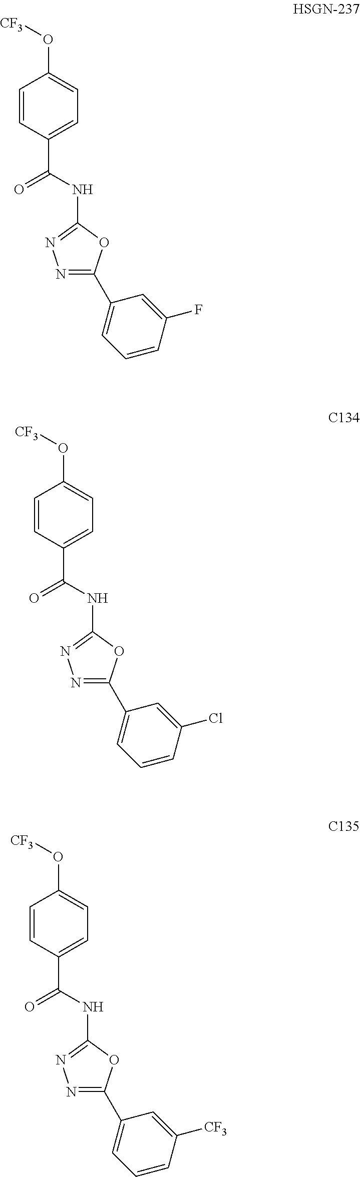 [SMILES: O=C(Nc1nnc(-c2cccc(F)c2)o1)c1ccc(OC(F)(F)F)cc1 ; O=C(Nc1nnc(-c2cccc(Cl)c2)o1)c1ccc(OC(F)(F)F)cc1 ; O=C(Nc1nnc(-c2cccc(C(F)(F)F)c2)o1)c1ccc(OC(F)(F)F)cc1]

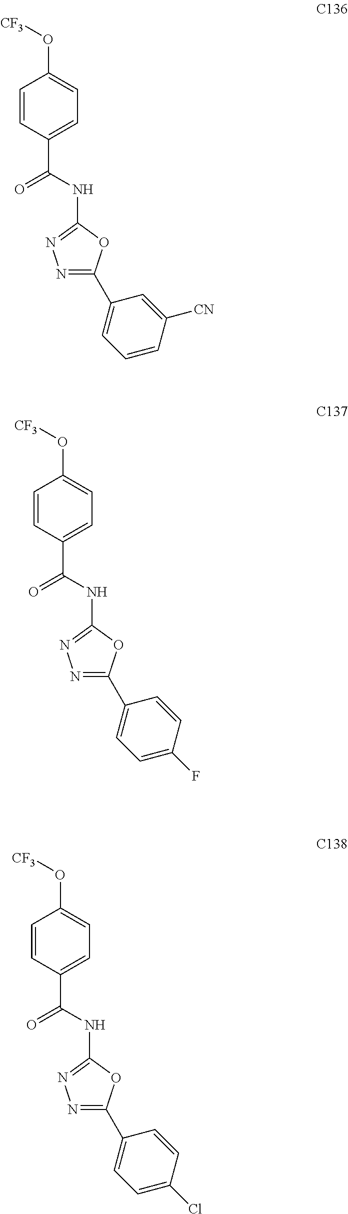 [SMILES: N#Cc1cccc(-c2nnc(NC(=O)c3ccc(OC(F)(F)F)cc3)o2)c1 ; O=C(Nc1nnc(-c2ccc(F)cc2)o1)c1ccc(OC(F)(F)F)cc1 ; O=C(Nc1nnc(-c2ccc(Cl)cc2)o1)c1ccc(OC(F)(F)F)cc1]

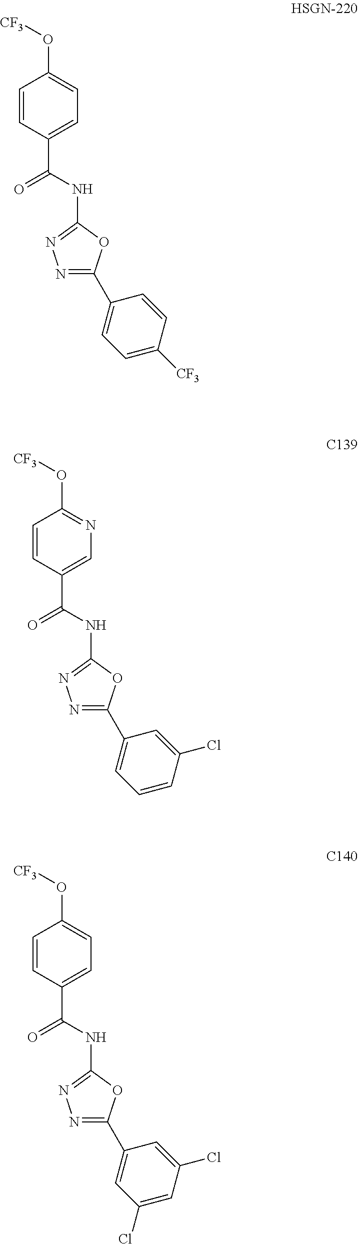 [SMILES: O=C(Nc1nnc(-c2ccc(C(F)(F)F)cc2)o1)c1ccc(OC(F)(F)F)cc1 ; O=C(Nc1nnc(-c2cccc(Cl)c2)o1)c1ccc(OC(F)(F)F)nc1 ; O=C(Nc1nnc(-c2cc(Cl)cc(Cl)c2)o1)c1ccc(OC(F)(F)F)cc1]

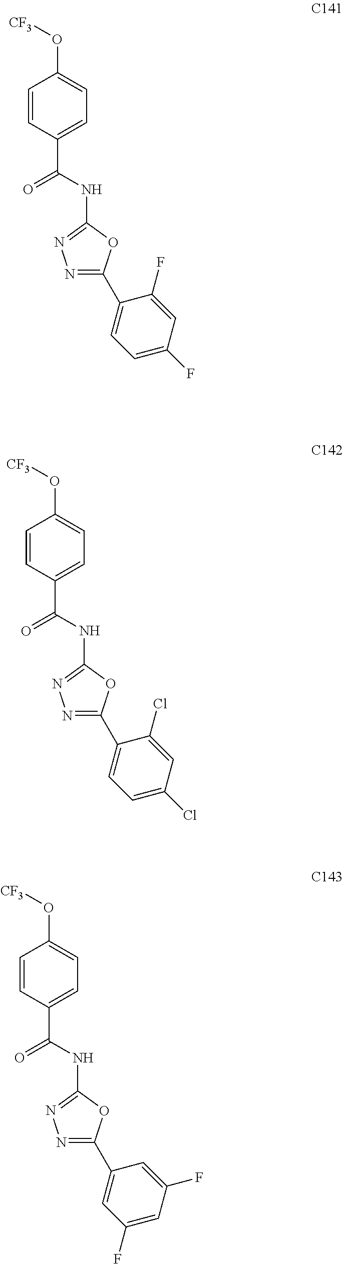 [SMILES: O=C(Nc1nnc(-c2ccc(F)cc2F)o1)c1ccc(OC(F)(F)F)cc1 ; O=C(Nc1nnc(-c2ccc(Cl)cc2Cl)o1)c1ccc(OC(F)(F)F)cc1 ; O=C(Nc1nnc(-c2cc(F)cc(F)c2)o1)c1ccc(OC(F)(F)F)cc1]

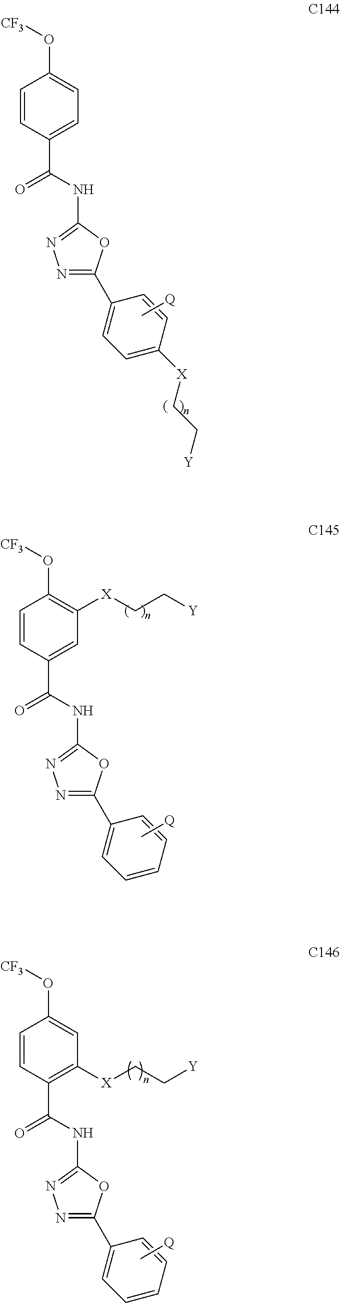

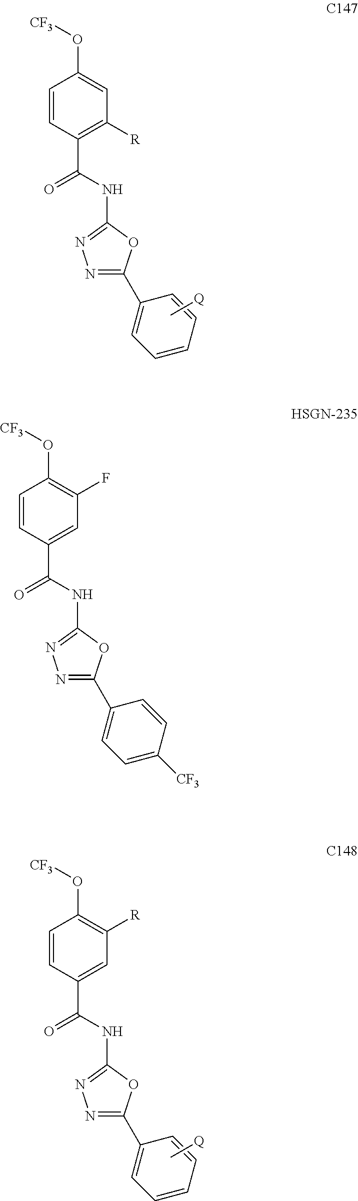

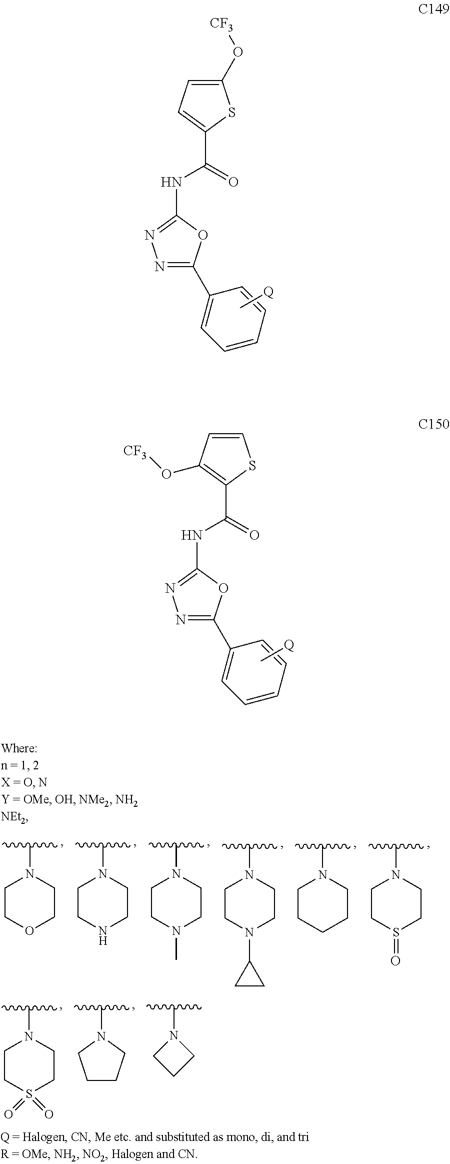

19. The compound according to claim 1, wherein the compound is one of the following:

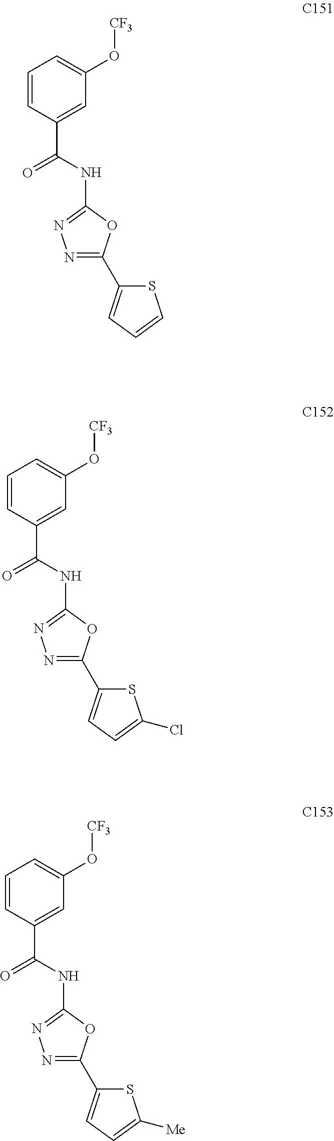 [SMILES: O=C(Nc1nnc(-c2cccs2)o1)c1cccc(OC(F)(F)F)c1 ; O=C(Nc1nnc(-c2ccc(Cl)s2)o1)c1cccc(OC(F)(F)F)c1 ; Cc1ccc(-c2nnc(NC(=O)c3cccc(OC(F)(F)F)c3)o2)s1]

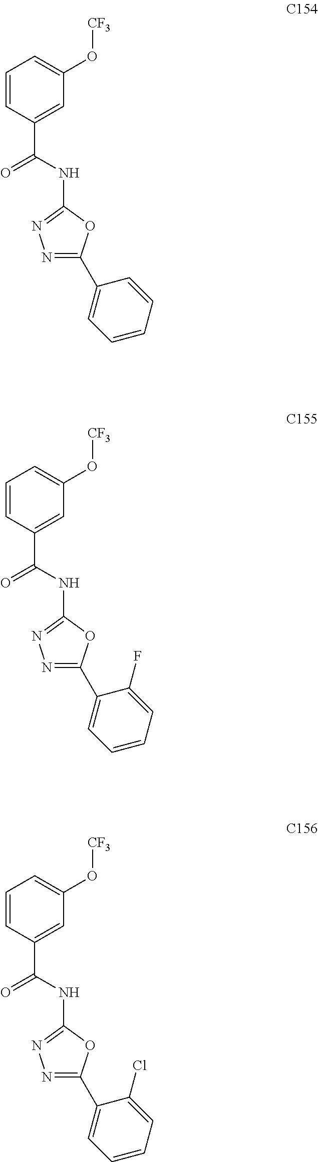 [SMILES: O=C(Nc1nnc(-c2ccccc2)o1)c1cccc(OC(F)(F)F)c1 ; O=C(Nc1nnc(-c2ccccc2F)o1)c1cccc(OC(F)(F)F)c1 ; O=C(Nc1nnc(-c2ccccc2Cl)o1)c1cccc(OC(F)(F)F)c1]

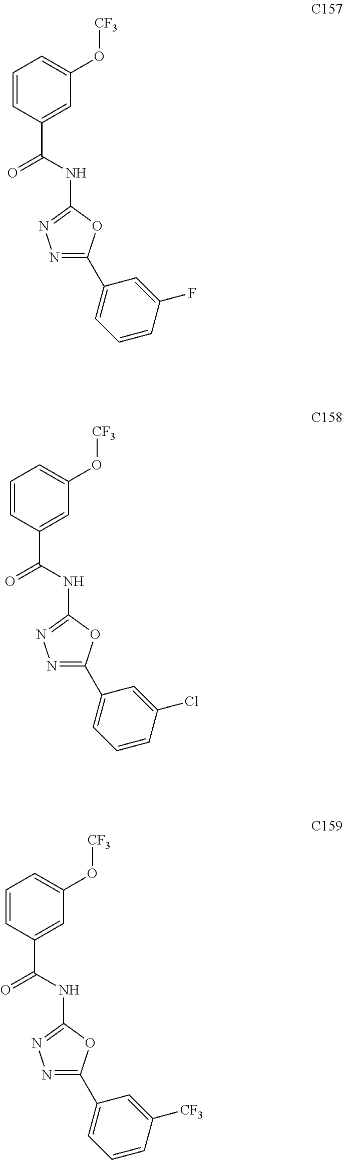 [SMILES: O=C(Nc1nnc(-c2cccc(F)c2)o1)c1cccc(OC(F)(F)F)c1 ; O=C(Nc1nnc(-c2cccc(Cl)c2)o1)c1cccc(OC(F)(F)F)c1 ; O=C(Nc1nnc(-c2cccc(C(F)(F)F)c2)o1)c1cccc(OC(F)(F)F)c1]

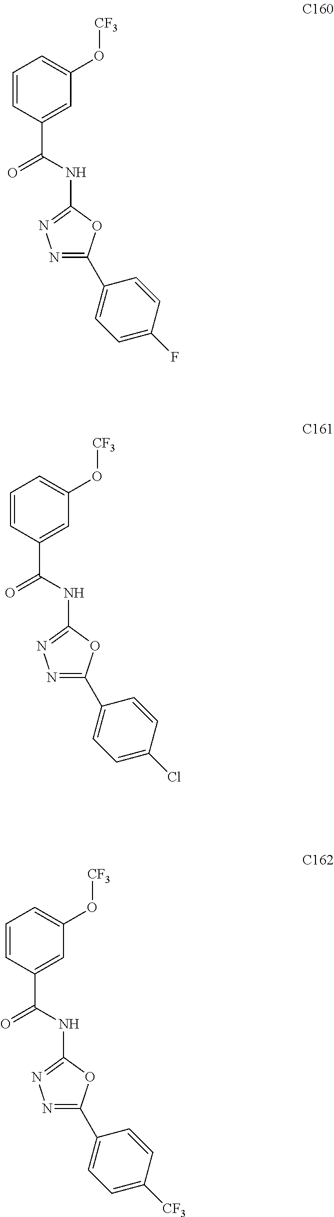 [SMILES: O=C(Nc1nnc(-c2ccc(F)cc2)o1)c1cccc(OC(F)(F)F)c1 ; O=C(Nc1nnc(-c2ccc(Cl)cc2)o1)c1cccc(OC(F)(F)F)c1 ; O=C(Nc1nnc(-c2ccc(C(F)(F)F)cc2)o1)c1cccc(OC(F)(F)F)c1]

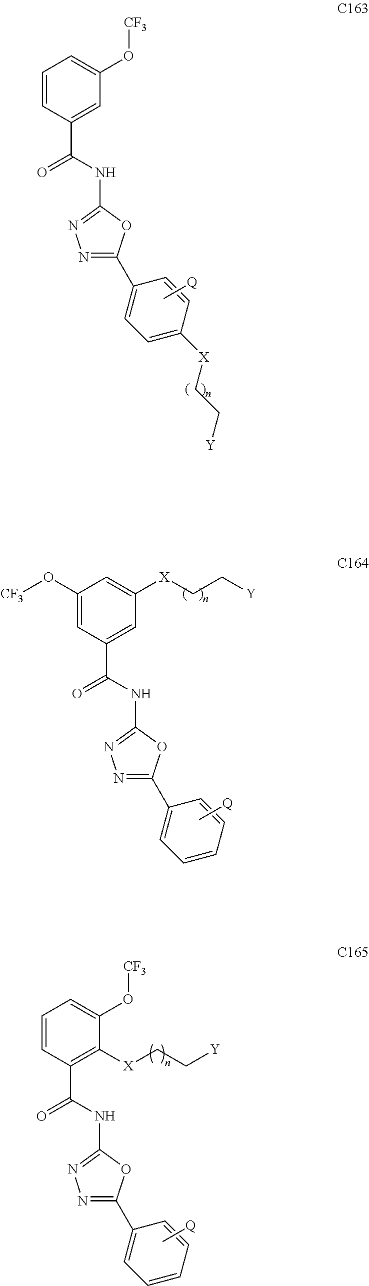

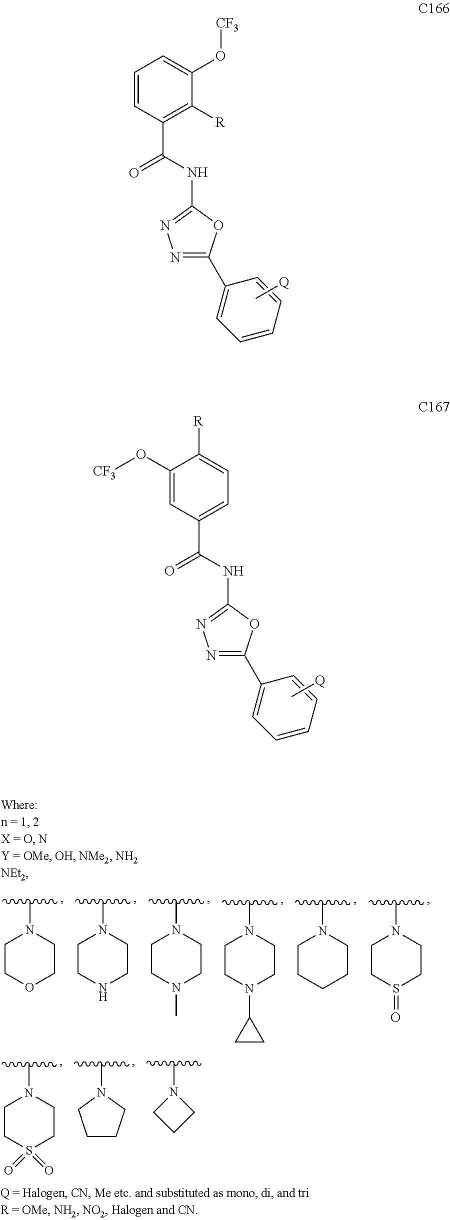

20. The compound according to claim 1, wherein the compound is one of the following:

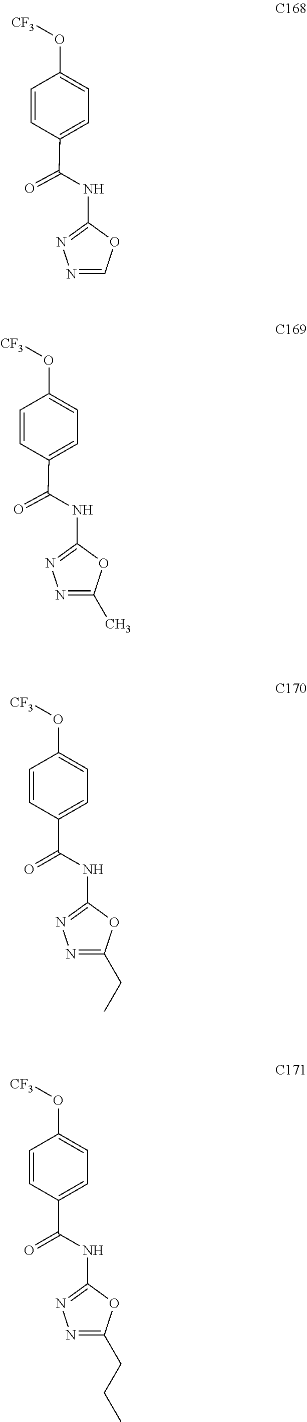 [SMILES: O=C(Nc1nnco1)c1ccc(OC(F)(F)F)cc1 ; Cc1nnc(NC(=O)c2ccc(OC(F)(F)F)cc2)o1 ; CCc1nnc(NC(=O)c2ccc(OC(F)(F)F)cc2)o1 ; CCCc1nnc(NC(=O)c2ccc(OC(F)(F)F)cc2)o1]

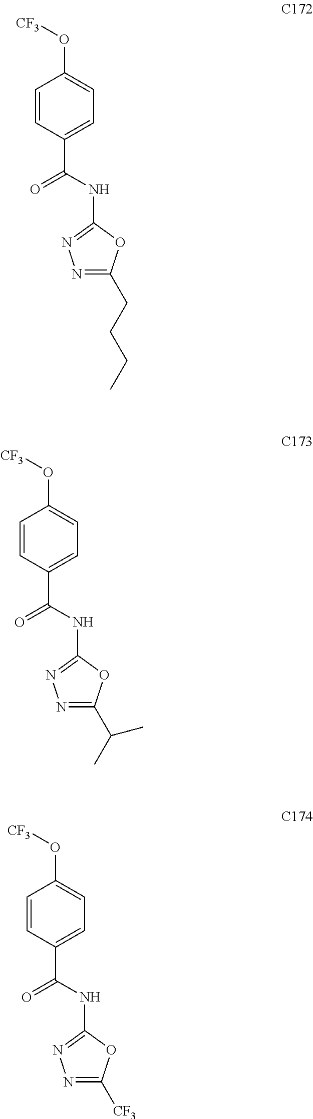 [SMILES: CCCCc1nnc(NC(=O)c2ccc(OC(F)(F)F)cc2)o1 ; CC(C)c1nnc(NC(=O)c2ccc(OC(F)(F)F)cc2)o1 ; O=C(Nc1nnc(C(F)(F)F)o1)c1ccc(OC(F)(F)F)cc1]

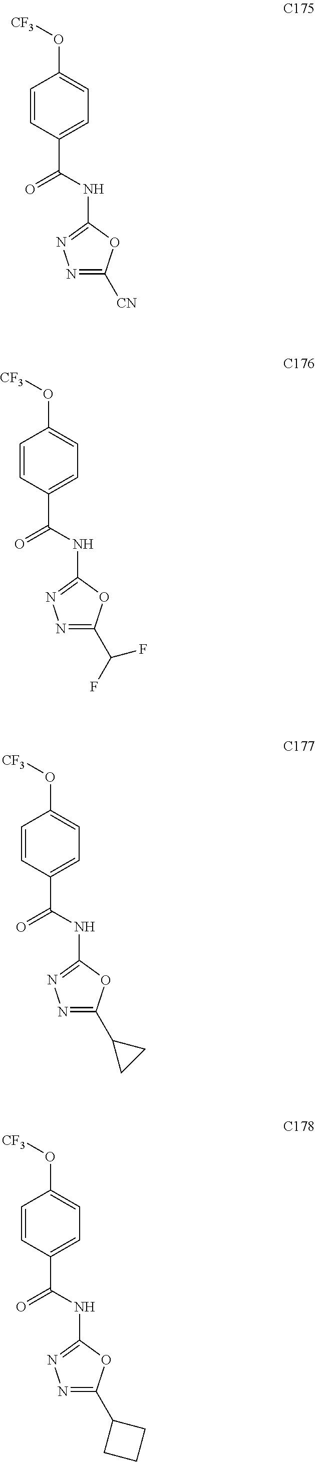 [SMILES: N#Cc1nnc(NC(=O)c2ccc(OC(F)(F)F)cc2)o1 ; O=C(Nc1nnc(C(F)F)o1)c1ccc(OC(F)(F)F)cc1 ; O=C(Nc1nnc(C2CC2)o1)c1ccc(OC(F)(F)F)cc1 ; O=C(Nc1nnc(C2CCC2)o1)c1ccc(OC(F)(F)F)cc1]

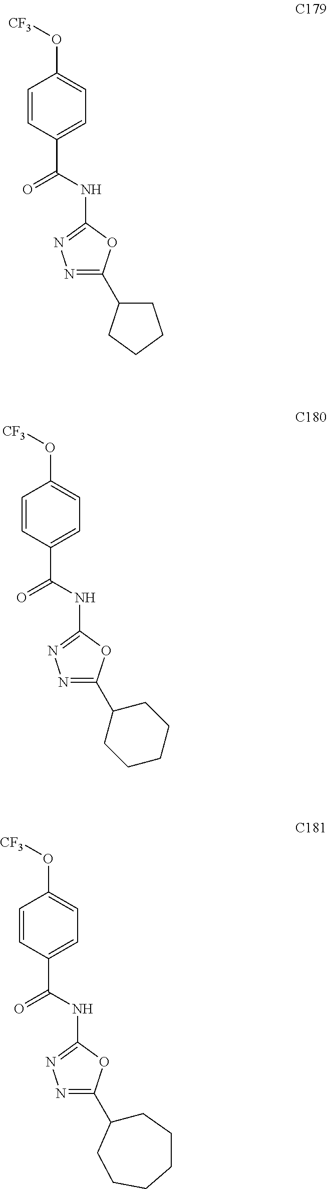 [SMILES: O=C(Nc1nnc(C2CCCC2)o1)c1ccc(OC(F)(F)F)cc1 ; O=C(Nc1nnc(C2CCCCC2)o1)c1ccc(OC(F)(F)F)cc1 ; O=C(Nc1nnc(C2CCCCCC2)o1)c1ccc(OC(F)(F)F)cc1]

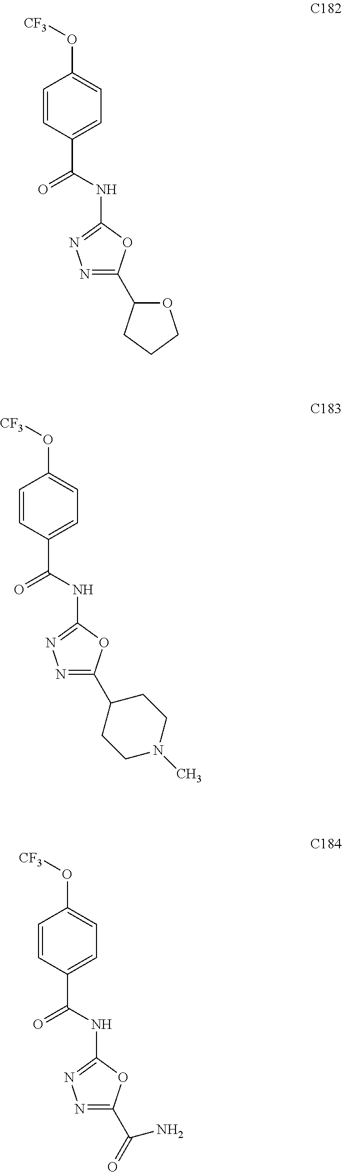 [SMILES: O=C(Nc1nnc(C2CCCO2)o1)c1ccc(OC(F)(F)F)cc1 ; CN1CCC(c2nnc(NC(=O)c3ccc(OC(F)(F)F)cc3)o2)CC1 ; NC(=O)c1nnc(NC(=O)c2ccc(OC(F)(F)F)cc2)o1]

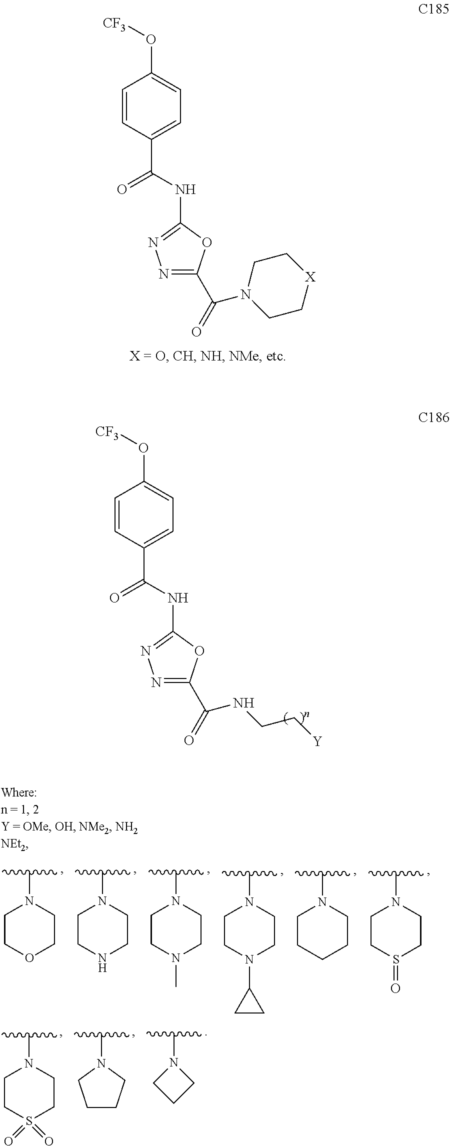

21. The compound according to claim 1, wherein the compound is one of the following:

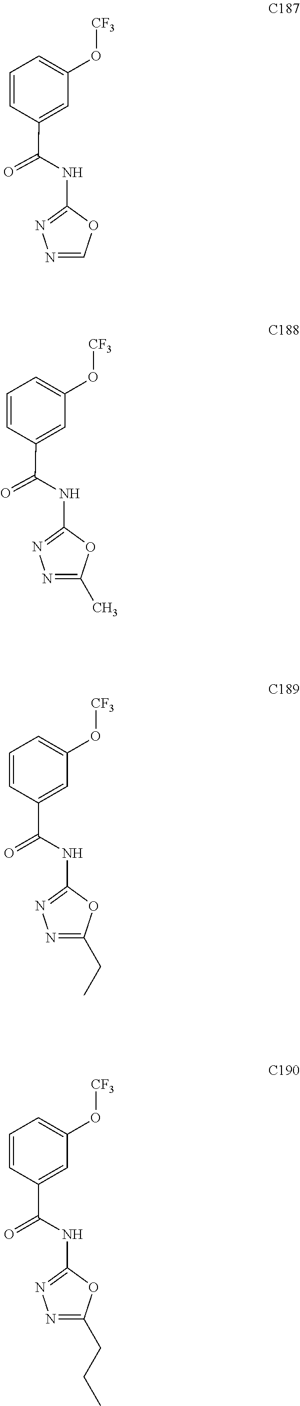 [SMILES: O=C(Nc1nnco1)c1cccc(OC(F)(F)F)c1 ; Cc1nnc(NC(=O)c2cccc(OC(F)(F)F)c2)o1 ; CCc1nnc(NC(=O)c2cccc(OC(F)(F)F)c2)o1 ; CCCc1nnc(NC(=O)c2cccc(OC(F)(F)F)c2)o1]

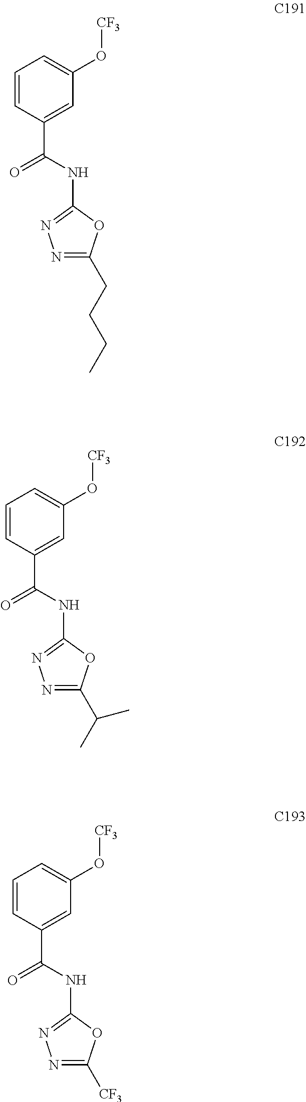 [SMILES: CCCCc1nnc(NC(=O)c2cccc(OC(F)(F)F)c2)o1 ; CC(C)c1nnc(NC(=O)c2cccc(OC(F)(F)F)c2)o1 ; O=C(Nc1nnc(C(F)(F)F)o1)c1cccc(OC(F)(F)F)c1]

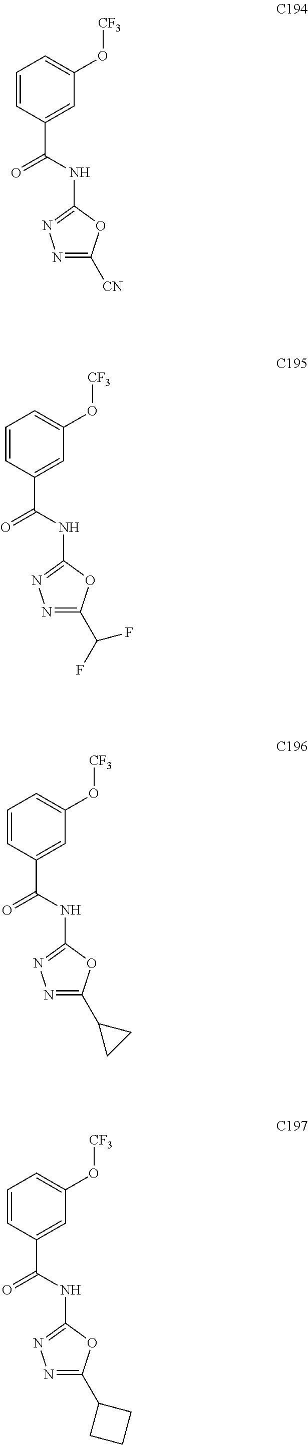 [SMILES: N#Cc1nnc(NC(=O)c2cccc(OC(F)(F)F)c2)o1 ; O=C(Nc1nnc(C(F)F)o1)c1cccc(OC(F)(F)F)c1 ; O=C(Nc1nnc(C2CC2)o1)c1cccc(OC(F)(F)F)c1 ; O=C(Nc1nnc(C2CCC2)o1)c1cccc(OC(F)(F)F)c1]

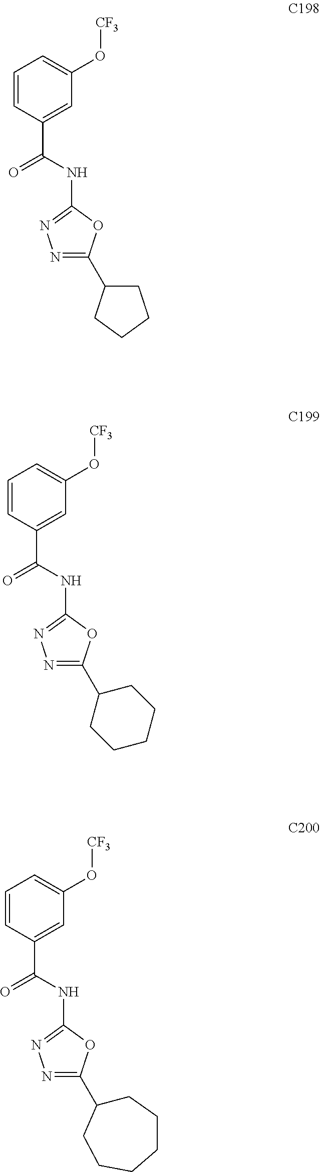 [SMILES: O=C(Nc1nnc(C2CCCC2)o1)c1cccc(OC(F)(F)F)c1 ; O=C(Nc1nnc(C2CCCCC2)o1)c1cccc(OC(F)(F)F)c1 ; O=C(Nc1nnc(C2CCCCCC2)o1)c1cccc(OC(F)(F)F)c1]

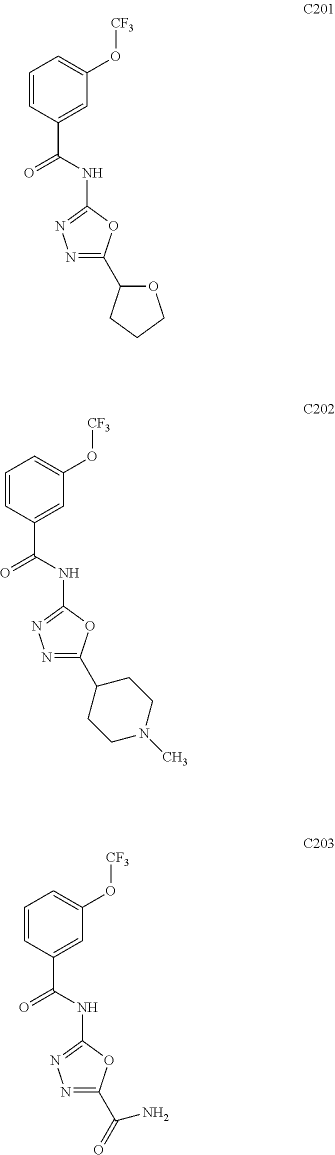 [SMILES: O=C(Nc1nnc(C2CCCO2)o1)c1cccc(OC(F)(F)F)c1 ; CN1CCC(c2nnc(NC(=O)c3cccc(OC(F)(F)F)c3)o2)CC1 ; NC(=O)c1nnc(NC(=O)c2cccc(OC(F)(F)F)c2)o1]

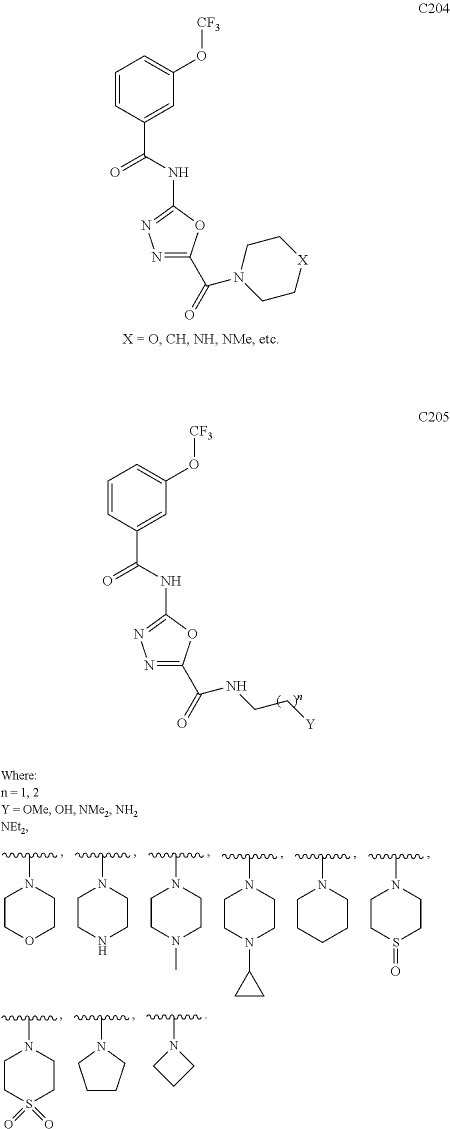

22. The compound according to claim 1, wherein the compound is one of the following:

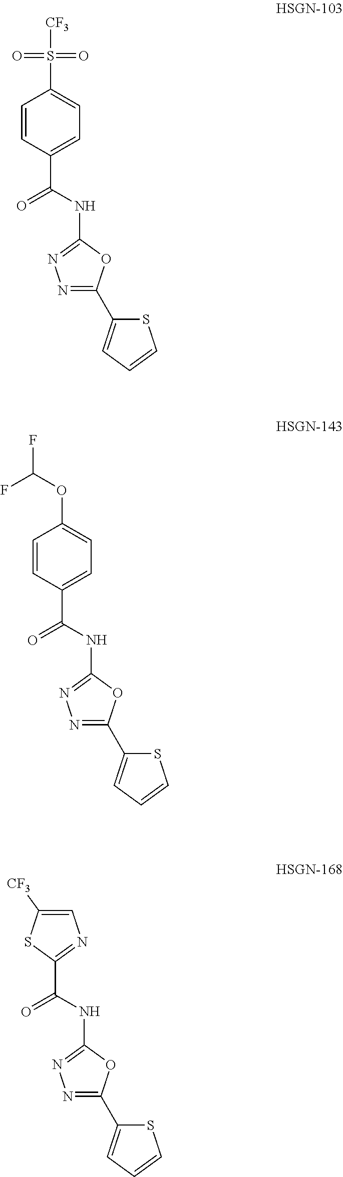 [SMILES: O=C(Nc1nnc(-c2cccs2)o1)c1ccc(S(=O)(=O)C(F)(F)F)cc1 ; O=C(Nc1nnc(-c2cccs2)o1)c1ccc(OC(F)F)cc1 ; O=C(Nc1nnc(-c2cccs2)o1)c1ncc(C(F)(F)F)s1]

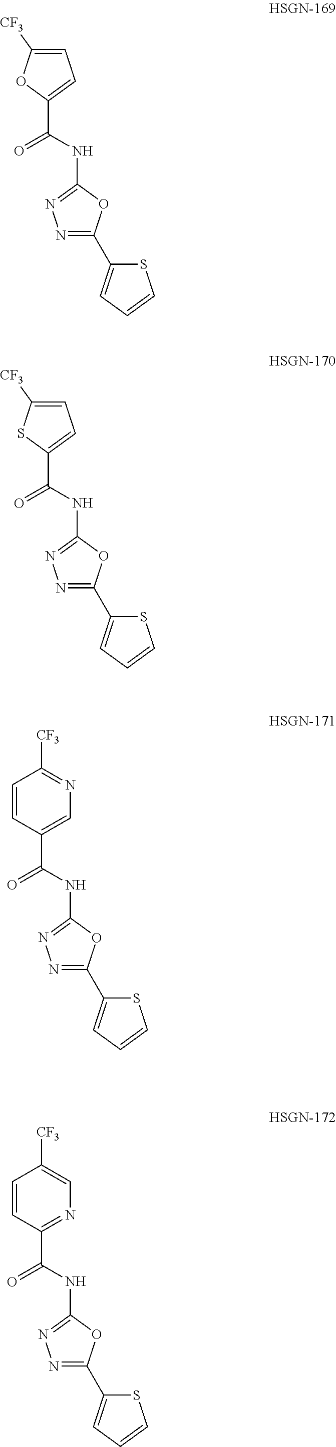 [SMILES: O=C(Nc1nnc(-c2cccs2)o1)c1ccc(C(F)(F)F)o1 ; O=C(Nc1nnc(-c2cccs2)o1)c1ccc(C(F)(F)F)s1 ; O=C(Nc1nnc(-c2cccs2)o1)c1ccc(C(F)(F)F)nc1 ; O=C(Nc1nnc(-c2cccs2)o1)c1ccc(C(F)(F)F)cn1]

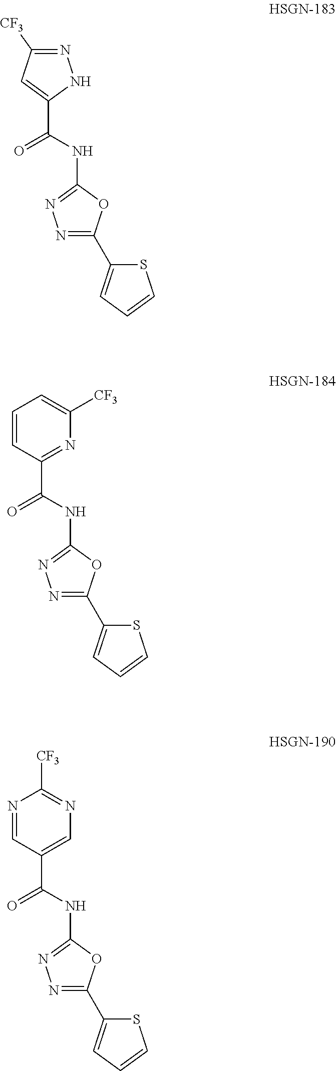 [SMILES: O=C(Nc1nnc(-c2cccs2)o1)c1cc(C(F)(F)F)n[nH]1 ; O=C(Nc1nnc(-c2cccs2)o1)c1cccc(C(F)(F)F)n1 ; O=C(Nc1nnc(-c2cccs2)o1)c1cnc(C(F)(F)F)nc1]

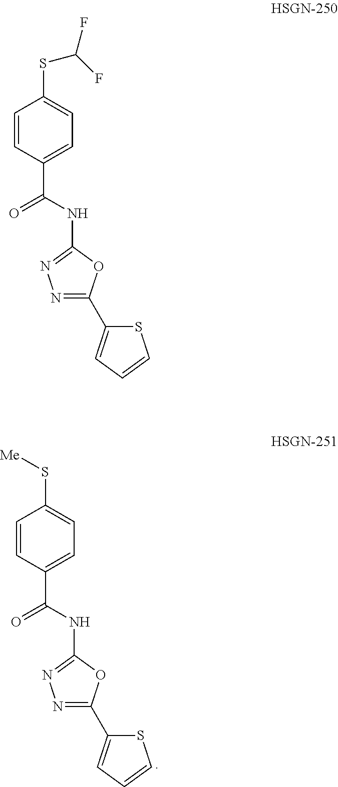 [SMILES: O=C(Nc1nnc(-c2cccs2)o1)c1ccc(SC(F)F)cc1 ; CSc1ccc(C(=O)Nc2nnc(-c3cccs3)o2)cc1]

23. A method for treating a patient with an infection comprising the step of administering a therapeutically effective amount of one or more compounds of claim 1, and one or more carriers, diluents, or excipients, to a patient in need of relief from said infection.

24. The method for treating a patient with an infection of claim 23, further comprising administering a therapeutically effective amount of the compound of claim 1 in combination with one or more other compounds of the same or different mode of action.

25. A drug conjugate comprising one or more compounds having a formula (I) 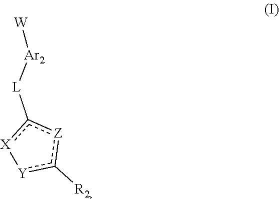
or a pharmaceutically acceptable salt thereof, wherein [symbol]
represents a single or double bond;
X, Y or Z is, independently, CH, O, S, N, or NR, wherein R is H, alkyl, aryl, heteroalkyl, or heteroaryl;
W is —CF₃, —OCF₃, —OCHF₂, —SCF₃, or —SF₅;
L is a linkage selected from the group comprising —NH—, NR—, —NHCO—, —CO—NH—, —NRCO—, —CO—NR—, —CO—, —NH—SO₂—, —NR—SO₂—, —O—, —S—, —S(═O)—, or —S(O₂)—;
R₂ is an optionally substituted aryl, heteroaryl, alkyl (linear, branched or cyclic), heteroalkyl (linear, branched or cyclic), amide, CN, urea, sulfone, sulfoxide, sulfonamide;
and
Ar₂ is an optionally substituted aryl or heteroaryl,
wherein the conjugate confers cell-type or tissue type targeting or the conjugate targets another pathway that synergizes the action of the compound of formula (I).

26. (canceled)

27. The drug conjugate of claim 25, wherein the drug conjugate is formulated into a pharmaceutical composition comprising one or more pharmaceutically acceptable diluents, excipients or carriers.

28-33. (canceled)
